(19) United States
(12) Patent Application Publication
(10) Pub. No.: US 2026/0193670 A1
(43) Pub. Date: Jul. 9, 2026

(54) ELEMENTS FOR DE-TARGETING GENE EXPRESSION IN LIVER

(71) Applicant: ENCODED THERAPEUTICS, INC., South San Francisco, CA (US)

(72) Inventors: Anne TANENHAUS, South San Francisco, CA (US); Mitchell LOPEZ, South San Francisco, CA (US); Tulasi Indrasinh SOLANKI, South San Francisco, CA (US); Serena LIU, South San Francisco, CA (US); Steven TAN, South San Francisco, CA (US); Ben ZHAO, South San Francisco, CA (US); John MCLAUGHLIN, South San Francisco, CA (US); Greg LUCEY, South San Francisco, CA (US); Raghavendra HOSUR, South San Francisco, CA (US)

(21) Appl. No.: 18/855,284

(22) PCT Filed: Apr. 14, 2023
(86) PCT No.: PCT/US2023/065801

§ 371 Date: Oct. 8, 2024

Related U.S. Application Data
(60) Provisional application No. 63/331,680, filed on Apr. 15, 2022.
(60) Provisional application No. 63/412,119, filed on Sep. 30, 2022.

(51) Int. Cl. C12N 15/86; A61K 48/00

(52) U.S. Cl. CPC C12N 15/86; A61K 48/00; C12N 2750/14143; C12N 2830/008

(57) ABSTRACT

The present disclosure provides sequences that reduce expression of an operably linked transgene in liver cells. In some aspects, the sequences can be employed in gene therapy vectors to detarget expression of a therapeutic transgene in liver cells of a subject.

### CROSS-REFERENCING

This application claims the benefit of U.S. provisional application Ser. No. 63/331,680, filed on Apr. 15, 2022, and 63/412,119, filed on Sep. 30, 2022, which applications are incorporated by reference herein.

### INCORPORATION BY REFERENCE OF SEQUENCE LISTING PROVIDED AS A SEQUENCE LISTING XML FILE

A Sequence Listing is provided herewith as a Sequence Listing XML, ENCO-005WO_SEQ_LIST, created on Apr. 13, 2023, and having a size of 39,274 bytes. The contents of the Sequence Listing XML are incorporated herein by reference in their entirety.

### INTRODUCTION

Gene therapy has enormous potential for the treatment of human diseases, particularly diseases that have an underlying genetic cause. In some gene therapy strategies, a therapeutic payload may be recombinantly expressed in a target cell that lacks or has a reduced amount or dysfunctional version of an essential protein. Expression of the therapeutic payload in the cells rescues those cells, thereby treating the disease. In one example, Tay-Sachs disease (which is recessively inherited and caused by mutations in the HEXA gene, which is on chromosome 15), can be successfully treated by expressing a functional version of hexA in the brain using adeno-associated virus (AAV) gene therapy.

One of the challenges in gene therapy is how to deliver a therapeutic payload to a specific tissue and not others. For example, some therapeutic payloads that have a positive effect in one tissue may have an adverse effect in another tissues. As such, administrating a gene therapy that targets diseased cells in one tissue may cause side-effects in another. In some cases, the clinical use of a gene therapy may even be limited by its off-site affects, rather than the on-site effects.

In view of the above, there is a general need for tools for increasing the tissue specificity of a gene therapy.

### SUMMARY

Provided herein, among other things, is a nucleic acid cassette comprising a transgene encoding an mRNA, wherein the mRNA comprises a sequence of (i) any of SEQ ID NOs. 1-17 or 39, (ii) a variant, functional fragment, or combination thereof, or (iii) a sequence at least 80%, 81%, 82%, 83%, 84%, 85%, 86%, 87%, 88%, 89%, 90%, 91%, 92%, 93%, 94%, 95%, 96%, 97%, 98%, or 99% identical to (i) or (ii). These sequences reduce the expression of the transgene in liver cells (e.g., relative to target cells such as neurons) and, as such, may be employed in a variety of gene therapy strategies that target cells that are not in the liver. In certain aspects, incorporation of one or more of these sequences results in an improved safety profile of a gene therapy by reducing or eliminating toxicity to liver cells caused by expression of the transgene in these cells.

In some embodiments, the nucleic acid cassette is an expression cassette, wherein the expression cassette may comprise, in operable linkage, a promoter, a coding sequence, a sequence encoding (i), (ii), or (iii) (also referred to as a liver de-targeting element), and a terminator. In some embodiments, at least one sequence present in the expression cassette is heterologous to another one of the sequences in the expression cassette. For example, in some embodiments, an expression cassette of the present disclosure comprises a promoter that is heterologous to an operably linked coding sequence. In some embodiments, the expression cassette may further comprise an enhancer and/or an intron.

In some embodiments, the promoter of the expression cassette may be selective for cells in a particular tissue (e.g., the target tissue, such as brain) but also drive transgene expression in the liver. In some embodiments, the promoter may be a CNS selective promoter, e.g., a promoter selected from the group consisting of: Ca2+/calmodulin-dependent kinase subunit a (CaMKII) promoters, synapsin I promoters, 67 kDa glutamic acid decarboxylase (GAD67) promoters, homeobox Dlx5/6 promoters, glutamate receptor 1 (GluR1) promoters, preprotachykinin 1 (Tac1) promoters, Neuron-specific enolase (NSE) promoters, dopaminergic receptor 1 (Drd1a) promoters, MAP1B promoters, Tα1 α-tubulin promoters, decarboxylase promoters, dopamine β-hydroxylase promoters, NCAM promoters, HES-5 promoters, α-internexin promoters, peripherin promoters, and GAP-43 promoters, and PaqR4 promoters.

In any embodiment, the sequence may be in a 3′ UTR, a 5′ UTR or an intron of the mRNA.

In any embodiment, the expression cassette may encode a therapeutic protein, e.g., SCN1A, SNC2A, SNC8A, SCN1B, SCN2B, KV3.1, KV3.2, KV3.3, STXBP1, UBE3A, or a transcription factor that activates endogenous expression of any of those proteins. In some embodiments, the therapeutic protein may be ALDH7A1, ARHGEF9, ARX, BRAT1, CACNA1A, CACNA1D, CACNB4, CDKL5, CHD2, CHRNA2, CHRNA4, CHRNB2, CLCN2, CLN, CLN2, DEPDC5, DNM1, FGF13, FMR1, FOLR1, FOXG1, GABRA1, GABRB3, GABRD, GABRG2, GRIN2A, GRIN2B, HCN1, HCN4, KCNQ2, KCNQ3, KCNT1, KV3.1, KV3.2, KV3.3, LGI1, MECP2, MEF2C, Myoclonin1/EFHC1, NPRL2, PCDH19, PLCB1, PNKP, POLG1, PRRT2, PTEN, SCN1A, SCN1B, SCN2A, SCN2B, SCN8A, SHANK3, SLC13A5, SLC25A22, SLC2A1, SLC6A1, SLC6A8, SPTAN1, ST3GAL3, STRADA, STXBP1, SYNGAP1, TBCID24, UBE3A, and WWOX, (ii) a protein having at least 90% sequence identity to (i), (iii) a functional fragment of (i) or (ii), or (iv) a transcription factor which activates expression of a gene from (i).

In some embodiments, the RNA transcript may comprise a combination of sequences of (i), (ii) and (iii).

Also provided is a vector comprising a cassette as summarized above. The vector may be a plasmid or viral vector, e.g., an adeno-associated virus (AAV) or lentiviral vector.

Also provided is an AAV or lentiviral particle or cell comprising a cassette as summarized above (which may be in single stranded form if it is packaged).

Also provided is an RNA encoded by the cassette summarized above.

A variety of methods are also provided. In some embodiments, the method may be for expressing a protein. In these embodiments, the method may comprise introducing an expression cassette as summarized above or an mRNA encoded thereby into an organism, wherein the sequence reduces the expression of the protein in liver cells in the organism.

Provided herein is a nucleic acid cassette comprising a therapeutic transgene encoding an mRNA, wherein the mRNA comprises a sequence of (i) any of SEQ ID NOs. 1-17 or 39, (ii) a variant, functional fragment, or combination thereof, or (iii) a sequence at least 80% identical to (i) or (ii).

In some embodiments, the mRNA comprises a sequence of at least 15 contiguous nucleotides of any of SEQ ID NOS. 1-17 or 39 that decreases expression in liver cells.

In some embodiments, the mRNA further comprises a second sequence of (i), (ii) or (iii).

In some embodiments, the mRNA further comprises a third sequence of (i), (ii) or (iii).

In some embodiments, the mRNA further comprises a fourth sequence of (i), (ii) or (iii).

In some embodiments, the mRNA comprises five or more sequences of (i), (ii) or (iii).

In some embodiments, the mRNA comprises two or more copies of a sequence of: (i), (ii) or (iii).

In some embodiments, the mRNA comprises three or more copies of a sequence of: (i), (ii) or (iii).

In some embodiments, the mRNA comprises four or more copies of a sequence of: (i), (ii) or (iii).

In some embodiments, the mRNA comprises five or more copies of a sequence of: (i), (ii) or (iii).

In some embodiments, the sequence of (i), (ii), or (iii) is located in one or more of: a 3′ UTR region of the mRNA, a 5′ UTR of the mRNA or an intron of the mRNA.

In some embodiments, the sequence of (i), (ii), or (iii) is located in a 3′ UTR region of the mRNA.

In some embodiments, the sequence of (i), (ii), or (iii) is located in a 5′ UTR region of the mRNA.

In some embodiments, the sequence of (i), (ii), or (iii) is located in an intron of the mRNA.

In some embodiments, the nucleic acid cassette is non-naturally occurring.

In some embodiments, the nucleic acid cassette comprises a CNS selective promoter.

In some embodiments, the CNS selective promoter is selected from the group consisting of: Ca2+/calmodulin-dependent kinase subunit a (CaMKII) promoters, synapsin I promoters, 67 kDa glutamic acid decarboxylase (GAD67) promoters, homeobox Dlx5/6 promoters, glutamate receptor 1 (GluR1) promoters, preprotachykinin 1 (Tac1) promoters, Neuron-specific enolase (NSE) promoters, dopaminergic receptor 1 (Drd1a) promoters, MAP1B promoters, Tα1 α-tubulin promoters, decarboxylase promoters, dopamine β-hydroxylase promoters, NCAM promoters, HES-5 promoters, α-internexin promoters, peripherin promoters, and GAP-43 promoters, and PaqR4 promoters.

In some embodiments, the nucleic acid cassette comprises an enhancer.

In some embodiments, the mRNA encodes a therapeutic protein that is associated with a neural disease or disorder.

In some embodiments, the neural disease or disorder is Alpers-Huttenlocher syndrome, Angelman syndrome, CDKL5 deficiency disorder, Dravet syndrome, Rett syndrome, Parkinson's disease and Parkinson's LIDS (side effect of Parkinson's medication), Alzheimer's disease, creatine transporter deficiency, FOXG1 syndrome, fragile X syndrome, Phelan-McDermid syndrome, childhood absence epilepsy, childhood epilepsy centrotemporal spikes (benign rolandic epilepsy), early myoclonic encephalopathy (EME), epilepsy eyelid myoclonia (Jeavons syndrome), epilepsy of infancy with migrating focal seizures, epilepsy myoclonic absences, epileptic encephalopathy continuous spike and wave during sleep (CSWS), infantile spasms (West syndrome), juvenile myoclonic epilepsy, Landau-Kleffner syndrome, Lennox-Gastaut syndrome (LGS), myoclonic epilepsy in infancy, Ohtahara syndrome, Panayiotopoulos syndrome, progressive myoclonic epilepsy, reflex Epilepsy, self-limited familial and non-familial neonatal infantile seizures, self-limited late onset occipital epilepsy, Gastaut syndrome, epilepsy generalized tonic clonic seizures alone, genetic epilepsy with febrile seizures plus, juvenile absence epilepsy, myoclonic atonic epilepsy (Doose syndrome), sleep-related hypermotor epilepsy (SHE), febrile seizures, focal epilepsy, West syndrome, early onset epilepsy, benign familial infantile epilepsy, or attention deficit-hyperactivity disorder.

In some embodiments, the therapeutic protein is selected from (i): a protein encoded by a gene selected from: ALDH7A1, ARHGEF9, ARX, BRAT1, CACNA1A, CACNA1D, CACNB4, CDKL5, CHD2, CHRNA2, CHRNA4, CHRNB2, CLCN2, CLN, CLN2, DEPDC5, DNM1, FGF13, FMR1, FOLR1, FOXG1, GABRA1, GABRB3, GABRD, GABRG2, GRIN2A, GRIN2B, HCN1, HCN4, KCNQ2, KCNQ3, KCNT1, KV3.1, KV3.2, KV3.3, LGI1, MECP2, MEF2C, Myoclonin1/EFHC1, NPRL2, PCDH19, PLCB1, PNKP, POLG1, PRRT2, PTEN, SCN1A, SCN1B, SCN2A, SCN2B, SCN8A, SHANK3, SLC13A5, SLC25A22, SLC2A1, SLC6A1, SLC6A8, SPTAN1, ST3GAL3, STRADA, STXBP1, SYNGAP1, TBC1D24, UBE3A, and WWOX, (ii) a protein having at least 90% sequence identity to (i), (iii) a functional fragment of (i) or (ii), or (iv) a transcription factor which activates expression of a gene from (i).

In some embodiments, the mRNA comprises a sequence of (i) any of SEQ ID NOs. 1-17 or 39, (ii) a variant, functional fragment, or combination thereof, or (iii) a sequence at least 80% identical to (i) or (ii); the nucleic acid cassette comprises a CNS-selective promoter; and the mRNA encodes a therapeutic protein that is associated with a neural disease or disorder.

In some embodiments, the mRNA comprises a sequence of (i) any of SEQ ID NOs. 1-17 or 39, (ii) a variant, functional fragment, or combination thereof, or (iii) a sequence at least 80% identical to (i) or (ii); the nucleic acid cassette comprises a promoter selected from the group consisting of Ca2+/calmodulin-dependent kinase subunit a (CaMKII) promoters, synapsin I promoters, 67 kDa glutamic acid decarboxylase (GAD67) promoters, homeobox Dlx5/6 promoters, glutamate receptor 1 (GluR1) promoters, preprotachykinin 1 (Tac1) promoters, Neuron-specific enolase (NSE) promoters, dopaminergic receptor 1 (Drd1a) promoters, MAP1B promoters, Tα1 α-tubulin promoters, decarboxylase promoters, dopamine β-hydroxylase promoters, NCAM promoters, HES-5 promoters, α-internexin promoters, peripherin promoters, and GAP-43 promoters, and PaqR4 promoters; and the mRNA encodes a therapeutic protein encoded by a gene selected from: ALDH7A1, ARHGEF9, ARX, BRAT1, CACNA1A, CACNA1D, CACNB4, CDKL5, CHD2, CHRNA2, CHRNA4, CHRNB2, CLCN2, CLN, CLN2, DEPDC5, DNM1, FGF13, FMR1, FOLR1, FOXG1, GABRA1, GABRB3, GABRD, GABRG2, GRIN2A, GRIN2B, HCN1, HCN4, KCNQ2, KCNQ3, KCNT1, KV3.1, KV3.2, KV3.3, LGI1, MECP2, MEF2C, Myoclonin1/EFHC1, NPRL2, PCDH19, PLCB1, PNKP, POLG1, PRRT2, PTEN, SCN1A, SCN1B, SCN2A, SCN2B, SCN8A, SHANK3, SLC13A5, SLC25A22, SLC2A1, SLC6A1, SLC6A8, SPTAN1, ST3GAL3, STRADA, STXBP1, SYNGAP1, TBC1D24, UBE3A, and WWOX, (ii) a protein having at least 90% sequence identity to (i), (iii) a functional fragment of (i) or (ii), or (iv) a transcription factor which activates expression of a gene from (i).

In some embodiments, the sequence of (i), (ii) or (iii), results in decreased expression of a polypeptide encoded by the mRNA in liver cells as compared to expression of the polypeptide in liver cells from an otherwise equivalent mRNA without the sequence of (i), (ii) or (iii).

In some embodiments, the sequence of (i), (ii) or (iii), result in decreased expression of a polypeptide encoded by the mRNA in liver cells at a level that is at least 2 fold, at least 5 fold, or at least 10 fold as compared to expression of the polypeptide in liver cells from an otherwise equivalent mRNA without the sequence of (i), (ii) or (iii).

In some embodiments, the sequence of (i), (ii) or (iii), result in decreased expression of a polypeptide encoded by the mRNA in liver cells at a level that is at least 2%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% lower than expression of the polypeptide in liver cells from an otherwise equivalent mRNA without the sequence of (i), (ii) or (iii).

In some embodiments, the sequence of (i), (ii) or (iii), does not result in greatly decreased expression of a polypeptide encoded by the mRNA in target cells as compared to expression of the polypeptide in target cells from an otherwise equivalent mRNA without the sequence of (i), (ii) or (iii).

In some embodiments, the sequence of (i), (ii) or (iii), does not decrease expression of the polypeptide encoded by the mRNA in the target cells as compared to expression of the polypeptide in the target cells from an otherwise equivalent mRNA without the sequence of (i), (ii) or (iii).

In some embodiments, the sequence of (i), (ii) or (iii), result in expression of a polypeptide encoded by the mRNA in target cells at a level that is at least at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of the expression of the polypeptide in target cells from an otherwise equivalent mRNA without the sequence of (i), (ii) or (iii).

In some embodiments, the target cells are neural cells.

In some embodiments, the neural cells are cerebrum cells, brainstem cells, hippocampus cells or cerebellum cells.

In some embodiments, the neural cells are GABAergic cells.

In some embodiments, the GABAergic cells are parvalbumin expressing cells.

In some embodiments, the nucleic acid cassette is a linear construct or a vector.

In some embodiments, the vector is a plasmid.

In some embodiments, the vector is a viral vector.

In some embodiments, the viral vector is an adeno-associated virus (AAV) vector.

In some embodiments, the AAV is AAV1, AAV2, AAV3, AAV4, AAV5, AAV6, AAV7, AAV8, AAV9, or AAV-DJ.

In some embodiments, the AAV is an scAAV.

In some embodiments, the viral vector is a lentiviral vector.

Also provided is an mRNA with a sequence encoded by a nucleic acid cassette summarized above.

Also provided is a nucleic acid cassette comprising a transgene encoding an mRNA, wherein the mRNA encodes a therapeutic protein and comprises a miRNA binding site for a miRNA selected from miR-22-3p, miR-1258-5p, miR-5589-3p, miR-17-5p, miR-203a, miR-122-3p, miR-93-5p, miR-122-5p or a complement thereof.

In some embodiments, the nucleic acid cassette may comprise miRNA binding sites for two or more miRNAs selected from miR-22-3p, miR-1258-5p, miR-5589-3p, miR-17-5p, miR-203a, miR-122-3p, miR-93-5p, miR-122-5p or a complement thereof.

In some embodiments, the nucleic acid cassette may comprise miRNA binding sites for three or more miRNAs selected from miR-22-3p, miR-1258-5p, miR-5589-3p, miR-17-5p, miR-203a, miR-122-3p, miR-93-5p, miR-122-5p or a complement thereof.

In some embodiments, the nucleic acid cassette may comprise two miRNA binding sites for a miRNA selected from miR-22-3p, miR-1258-5p, miR-5589-3p, miR-17-5p, miR-203a, miR-122-3p, miR-93-5p, miR-122-5p or a complement thereof.

In some embodiments, the nucleic acid cassette may comprise three miRNA binding sites for a miRNA selected from miR-22-3p, miR-1258-5p, miR-5589-3p, miR-17-5p, miR-203a, miR-122-3p, miR-93-5p, miR-122-5p or a complement thereof.

In some embodiments, the nucleic acid cassette may comprise four miRNA binding sites for a miRNA selected from miR-22-3p, miR-1258-5p, miR-5589-3p, miR-17-5p, miR-203a, miR-122-3p, miR-93-5p, miR-122-5p or a complement thereof.

In some embodiments, the nucleic acid cassette may comprise more than four miRNA binding sites for a miRNA selected from miR-22-3p, miR-1258-5p, miR-5589-3p, miR-17-5p, miR-203a, miR-122-3p, miR-93-5p, miR-122-5p or a complement thereof.

In some embodiments, the miRNA is miR-22-3p.

In some embodiments, the miRNA is miR-1258-5p.

In some embodiments, the miRNA is miR-5589-3p.

In some embodiments, the miRNA is miR-17-5p.

In some embodiments, the miRNA is miR-203a.

In some embodiments, the miRNA is miR-122-3p.

In some embodiments, the miRNA is miR-93-5p.

In some embodiments, the miRNA is miR-122-5p.

In some embodiments, the mRNA additionally comprises a sequence of at least 10 contiguous nucleotides of any of SEQ ID NOS. 12-17 that decreases expression in liver cells.

In some embodiments, the mRNA additionally comprises at least two sequences of at least 20 contiguous nucleotides of any of SEQ ID NOS. 12-17 that decreases expression in liver cells.

In some embodiments, the miRNA binding site is located in one or more of: a 3′ UTR region of the mRNA, a 5′ UTR of the mRNA or an intron of the mRNA.

In some embodiments, the miRNA binding site is located in a 3′ UTR region of the mRNA.

In some embodiments, the miRNA binding site is located in a 5′ UTR region of the mRNA.

In some embodiments, the miRNA binding site is located in an intron of the mRNA.

In some embodiments, the nucleic acid cassette is non-naturally occurring.

In some embodiments, the nucleic acid cassette comprises a promoter, optionally a neural selective promoter.

In some embodiments, the CNS selective promoter is selected from the group consisting of: Ca2+/calmodulin-dependent kinase subunit a (CaMKII) promoters, synapsin I promoters, 67 kDa glutamic acid decarboxylase (GAD67) promoters, homeobox Dlx5/6 promoters, glutamate receptor 1 (GluR1) promoters, preprotachykinin 1 (Tac1) promoters, Neuron-specific enolase (NSE) promoters, dopaminergic receptor 1 (Drd1a) promoters, MAP1B promoters, Tα1 α-tubulin promoters, decarboxylase promoters, dopamine β-hydroxylase promoters, NCAM promoters, HES-5 promoters, α-internexin promoters, peripherin promoters, and GAP-43 promoters, and PaqR4 promoters.

In some embodiments, the nucleic acid cassette comprises an enhancer.

In some embodiments, the mRNA encodes a therapeutic protein.

In some embodiments, the therapeutic protein is a protein associated with a neural disease or disorder, optionally Alpers-Huttenlocher Syndrome, Angelman Syndrome, CDKL5 Deficiency Disorder, Dravet Syndrome, Rett Syndrome, Parkinson's Disease and Parkinson's LIDS (side effect of Parkinson's medication), Alzheimer's disease, Creatine Transporter Deficiency, FOXG1 Syndrome, Fragile X Syndrome, Phelan-McDermid Syndrome, Childhood Absence Epilepsy, Childhood Epilepsy Centrotemporal Spikes (Benign Rolandic Epilepsy), Dravet Syndrome, Early Myoclonic Encephalopathy (EME), Epilepsy Eyelid Myoclonia Jeavons Syndrome, Epilepsy of Infancy with Migrating Focal Seizures, Epilepsy Myoclonic Absences, Epileptic Encephalopathy Continuous Spike and Wave During Sleep CSWS, Infantile Spasms (West Syndrome), Juvenile Myoclonic Epilepsy, Landau-Kleffner Syndrome, Lennox-Gastaut Syndrome (LGS), Myoclonic Epilepsy in Infancy, Ohtahara Syndrome, Panayiotopoulos Syndrome, Progressive Myoclonic Epilepsies, Reflex Epilepsies, Self-Limited Familial and Non-Familial Neonatal Infantile Seizures, Self-Limited Late Onset Occipital Epilepsy Gastaut Syndrome, Epilepsy Generalized Tonic Clonic Seizures Alone, Genetic Epilepsy with Febrile Seizures Plus, Juvenile Absence Epilepsy, Myoclonic Atonic Epilepsy Doose Syndrome, Sleep-related Hypermotor Epilepsy (SHE), febrile seizures, focal epilepsy, West Syndrome, Early Onset Epilepsy, Benign Familial Infantile Epilepsy, or Attention Deficit-Hyperactivity Disorder.

In some embodiments, the therapeutic transgene is selected from (a): a protein encoded by a gene selected from: ALDH7A1, ARHGEF9, ARX, BRAT1, CACNA1A, CACNA1D, CACNB4, CDKL5, CHD2, CHRNA2, CHRNA4, CHRNB2, CLCN2, CLN, CLN2, DEPDC5, DNM1, FGF13, FMR1, FOLR1, FOXG1, GABRA1, GABRB3, GABRD, GABRG2, GRIN2A, GRIN2B, HCN1, HCN4, KCNQ2, KCNQ3, KCNT1, KV3.1, KV3.2, KV3.3, LGI1, MECP2, MEF2C, Myoclonin1/EFHC1, NPRL2, PCDH19, PLCB1, PNKP, POLG1, PRRT2, PTEN, SCN1A, SCN1B, SCN2A, SCN2B, SCN8A, SHANK3, SLC13A5, SLC25A22, SLC2A1, SLC6A1, SLC6A8, SPTAN1, ST3GAL3, STRADA, STXBP1, SYNGAP1, TBC1D24, UBE3A, and WWOX, (b) a protein having at least 90% sequence identity to (a), (c) a functional fragment of (a) or (b), or (d) a transcription factor which activates expression of a gene from (a).

In some embodiments, the mRNA comprises a sequence of (i) any of SEQ ID NOs. 1-17 or 39, (ii) a variant, functional fragment, or combination thereof, or (iii) a sequence at least 80% identical to (i) or (ii); the nucleic acid cassette comprises a CNS-selective promoter; and the mRNA encodes a therapeutic protein that is associated with a neural disease or disorder. In some embodiments, the mRNA comprises a sequence of (i) any of SEQ ID NOs. 1-17 or 39, (ii) a variant, functional fragment, or combination thereof, or (iii) a sequence at least 80% identical to (i) or (ii) the nucleic acid cassette comprises a promoter selected from the group consisting of Ca2+/calmodulin-dependent kinase subunit a (CaMKII) promoters, synapsin I promoters, 67 kDa glutamic acid decarboxylase (GAD67) promoters, homeobox Dlx5/6 promoters, glutamate receptor 1 (GluR1) promoters, preprotachykinin 1 (Tac1) promoters, Neuron-specific enolase (NSE) promoters, dopaminergic receptor 1 (Drd1a) promoters, MAP1B promoters, Tα1 α-tubulin promoters, decarboxylase promoters, dopamine β-hydroxylase promoters, NCAM promoters, HES-5 promoters, α-internexin promoters, peripherin promoters, and GAP-43 promoters, and PaqR4 promoters; and the mRNA encodes a therapeutic protein encoded by a gene selected from: ALDH7A1, ARHGEF9, ARX, BRAT1, CACNA1A, CACNA1D, CACNB4, CDKL5, CHD2, CHRNA2, CHRNA4, CHRNB2, CLCN2, CLN, CLN2, DEPDC5, DNM1, FGF13, FMR1, FOLR1, FOXG1, GABRA1, GABRB3, GABRD, GABRG2, GRIN2A, GRIN2B, HCN1, HCN4, KCNQ2, KCNQ3, KCNT1, KV3.1, KV3.2, KV3.3, LGI1, MECP2, MEF2C, Myoclonin1/EFHC1, NPRL2, PCDH19, PLCB1, PNKP, POLG1, PRRT2, PTEN, SCN1A, SCN1B, SCN2A, SCN2B, SCN8A, SHANK3, SLC13A5, SLC25A22, SLC2A1, SLC6A1, SLC6A8, SPTAN1, ST3GAL3, STRADA, STXBP1, SYNGAP1, TBC1D24, UBE3A, and WWOX, (ii) a protein having at least 90% sequence identity to (i), (iii) a functional fragment of (i) or (ii), or (iv) a transcription factor which activates expression of a gene from (i).

In some embodiments, the miRNA binding site results in decreased expression of a polypeptide encoded by the mRNA in liver cells as compared to expression of the polypeptide in liver cells from an otherwise equivalent mRNA without the miRNA binding site.

In some embodiments, the miRNA binding site results in decreased expression of a polypeptide encoded by the mRNA in liver cells at a level that is at least 2 fold, at least 5 fold, or at least 10 fold as compared to expression of the polypeptide in liver cells from an otherwise equivalent mRNA without the miRNA binding site.

In some embodiments, the miRNA binding site results in decreased expression of a polypeptide encoded by the mRNA in liver cells at a level that is at least 2%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% lower than expression of the polypeptide in liver cells from an otherwise equivalent mRNA without the miRNA binding site.

In some embodiments, the sequence of (i), (ii) or (iii), does not result in greatly decreased expression of a polypeptide encoded by the mRNA in target cells as compared to expression of the polypeptide in target cells from an otherwise equivalent mRNA without the sequence of (i), (ii) or (iii).

In some embodiments, the sequence of (i), (ii) or (iii), does not decrease expression of the polypeptide encoded by the mRNA in the target cells as compared to expression of the polypeptide in the target cells from an otherwise equivalent mRNA without the sequence of (i), (ii) or (iii).

In some embodiments, the sequence of (i), (ii) or (iii), result in expression of a polypeptide encoded by the mRNA in target cells at a level that is at least at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of the expression of the polypeptide in target cells from an otherwise equivalent mRNA without the sequence of (i), (ii) or (iii).

In some embodiments, the target cells are neural cells.

In some embodiments, the neural cells are cerebrum cells, brainstem cells, hippocampus cells or cerebellum cells.

In some embodiments, the neural cells are GABAergic cells.

In some embodiments, the GABAergic cells are parvalbumin expressing cells.

In some embodiments, the nucleic acid cassette is a linear construct or a vector.

In some embodiments, the vector is a plasmid.

In some embodiments, the vector is a viral vector.

In some embodiments, the viral vector is an adeno-associated virus (AAV) vector.

In some embodiments, the AAV is AAV1, AAV2, AAV3, AAV4, AAV5, AAV6, AAV7, AAV8, AAV9, or AAV-DJ.

In some embodiments, the AAV is an scAAV.

In some embodiments, the viral vector is a lentiviral vector.

Also provided is an mRNA encoded by a nucleic acid cassette of any embodiment summarized above.

In some embodiments, the mRNA encodes a polypeptide.

In some embodiments, the polypeptide is a therapeutic protein.

Also provided is an mRNA with a sequence encoded by the nucleic acid cassette of any embodiment summarized above.

Also provided is a method of decreasing liver expression of a therapeutic protein encoded by an mRNA while maintaining expression of the therapeutic protein in a target tissue, the method comprising including a sequence of (i) any of SEQ ID NOs. 1-17 or 39, (ii) a variant, functional fragment, or a combination thereof, or (iii) a sequence at least 80% identical to (i) or (ii) in the mRNA.

In some embodiments, the mRNA further comprises a second sequence of (i), (ii) or (iii).

In some embodiments, the mRNA further comprises a third sequence of (i), (ii) or (iii).

In some embodiments, the mRNA further comprises a fourth sequence of (i), (ii) or (iii).

In some embodiments, the mRNA comprises five or more sequences of (i), (ii) or (iii).

In some embodiments, the mRNA comprises two or more copies of a sequence of: (i), (ii) or (iii).

In some embodiments, the mRNA comprises three or more copies of a sequence of: (i), (ii) or (iii).

In some embodiments, the mRNA comprises four or more copies of a sequence of: (i), (ii) or (iii).

In some embodiments, the mRNA comprises five or more copies of a sequence of: (i), (ii) or (iii).

In some embodiments, the mRNA comprises at least 10 contiguous nucleotides of any of SEQ ID NOS. 1-17 or 39 that decreases expression in liver cells.

In some embodiments, the sequence of (i), (ii), or (iii) is located in one or more of: a 3′ UTR region of the mRNA, a 5′ UTR of the mRNA or an intron of the mRNA.

In some embodiments, the sequence of (i), (ii), or (iii) is located in a 3′ UTR region of the mRNA.

In some embodiments, the sequence of (i), (ii), or (iii) is located in a 5′ UTR region of the mRNA.

In some embodiments, the sequence of (i), (ii), or (iii) is located in an intron of the mRNA.

In some embodiments, the method comprises administering a nucleic acid encoding the mRNA to a subject.

In some embodiments, the administering is a systemically administering.

In some embodiments, the administering is a locally administering.

In some embodiments, the nucleic acid is administered locally into to the brain or CNS tissue.

In some embodiments, the administering by intraparenchymal, intrathecal, intra-cisterna magna, intracerebroventricular or intracranial administration.

In some embodiments, the therapeutic protein is a protein associated with a neural disease or disorder.

In some embodiments, the therapeutic protein is selected from (i): a protein encoded by a gene selected from: ALDH7A1, ARHGEF9, ARX, BRAT1, CACNA1A, CACNA1D, CACNB4, CDKL5, CHD2, CHRNA2, CHRNA4, CHRNB2, CLCN2, CLN, CLN2, DEPDC5, DNM1, FGF13, FMR1, FOLR1, FOXG1, GABRA1, GABRB3, GABRD, GABRG2, GRIN2A, GRIN2B, HCN1, HCN4, KCNQ2, KCNQ3, KCNT1, KV3.1, KV3.2, KV3.3, LGI1, MECP2, MEF2C, Myoclonin1/EFHC1, NPRL2, PCDH19, PLCB1, PNKP, POLG1, PRRT2, PTEN, SCN1A, SCN1B, SCN2A, SCN2B, SCN8A, SHANK3, SLC13A5, SLC25A22, SLC2A1, SLC6A1, SLC6A8, SPTAN1, ST3GAL3, STRADA, STXBP1, SYNGAP1, TBC1D24, UBE3A, and WWOX, (ii) a protein having at least 90% sequence identity to (i), (iii) a functional fragment of (i) or (ii), or (iv) a transcription factor which activates expression of a gene from (i).

In some embodiments, the subject has a neural disease or disorder

In some embodiments, the subject has Alpers-Huttenlocher syndrome, Angelman syndrome, CDKL5 deficiency disorder, Dravet syndrome, Rett syndrome, Parkinson's disease and Parkinson's LIDS (side effect of Parkinson's medication), Alzheimer's disease, creatine transporter deficiency, FOXG1 syndrome, fragile X syndrome, Phelan-McDermid syndrome, childhood absence epilepsy, childhood epilepsy centrotemporal spikes (benign rolandic epilepsy), early myoclonic encephalopathy (EME), epilepsy eyelid myoclonia (Jeavons syndrome), epilepsy of infancy with migrating focal seizures, epilepsy myoclonic absences, epileptic encephalopathy continuous spike and wave during sleep (CSWS), infantile spasms (West syndrome), juvenile myoclonic epilepsy, Landau-Kleffner syndrome, Lennox-Gastaut syndrome (LGS), myoclonic epilepsy in infancy, Ohtahara syndrome, Panayiotopoulos syndrome, progressive myoclonic epilepsy, reflex Epilepsy, self-limited familial and non-familial neonatal infantile seizures, self-limited late onset occipital epilepsy, Gastaut syndrome, epilepsy generalized tonic clonic seizures alone, genetic epilepsy with febrile seizures plus, juvenile absence epilepsy, myoclonic atonic epilepsy (Doose syndrome), sleep-related hypermotor epilepsy (SHE), febrile seizures, focal epilepsy, West syndrome, early onset epilepsy, benign familial infantile epilepsy, or attention deficit-hyperactivity disorder.

In some embodiments, the nucleic acid cassette comprises a CNS selective promoter.

In some embodiments, the CNS selective promoter is selected from the group consisting of: Ca2+/calmodulin-dependent kinase subunit a (CaMKII) promoters, synapsin I promoters, 67 kDa glutamic acid decarboxylase (GAD67) promoters, homeobox Dlx5/6 promoters, glutamate receptor 1 (GluR1) promoters, preprotachykinin 1 (Tac) promoters, Neuron-specific enolase (NSE) promoters, dopaminergic receptor 1 (Drd1a) promoters, MAP1B promoters, Tα1 α-tubulin promoters, decarboxylase promoters, dopamine β-hydroxylase promoters, NCAM promoters, HES-5 promoters, α-internexin promoters, peripherin promoters, and GAP-43 promoters, and PaqR4 promoters.

In some embodiments, the mRNA comprises a sequence of (i) any of SEQ ID NOs. 1-17 or 39, (ii) a variant, functional fragment, or combination thereof, or (iii) a sequence at least 80% identical to (i) or (ii); the nucleic acid cassette comprises a CNS-selective promoter; and the mRNA encodes a therapeutic protein that is associated with a neural disease or disorder.

In some embodiments, the mRNA comprises a sequence of (i) any of SEQ ID NOs. 1-17 or 39, (ii) a variant, functional fragment, or combination thereof, or (iii) a sequence at least 80% identical to (i) or (ii); the nucleic acid cassette comprises a promoter selected from the group consisting of Ca2+/calmodulin-dependent kinase subunit a (CaMKII) promoters, synapsin I promoters, 67 kDa glutamic acid decarboxylase (GAD67) promoters, homeobox Dlx5/6 promoters, glutamate receptor 1 (GluR1) promoters, preprotachykinin 1 (Tac1) promoters, Neuron-specific enolase (NSE) promoters, dopaminergic receptor 1 (Drd1a) promoters, MAP1B promoters, Tα1 α-tubulin promoters, decarboxylase promoters, dopamine β-hydroxylase promoters, NCAM promoters, HES-5 promoters, α-internexin promoters, peripherin promoters, and GAP-43 promoters, and PaqR4 promoters; and the mRNA encodes a therapeutic protein encoded by a gene selected from: ALDH7A1, ARHGEF9, ARX, BRAT1, CACNA1A, CACNA1D, CACNB4, CDKL5, CHD2, CHRNA2, CHRNA4, CHRNB2, CLCN2, CLN, CLN2, DEPDC5, DNM1, FGF13, FMR1, FOLR1, FOXG1, GABRA1, GABRB3, GABRD, GABRG2, GRIN2A, GRIN2B, HCN1, HCN4, KCNQ2, KCNQ3, KCNT1, KV3.1, KV3.2, KV3.3, LGI1, MECP2, MEF2C, Myoclonin1/EFHC1, NPRL2, PCDH19, PLCB1, PNKP, POLG1, PRRT2, PTEN, SCN1A, SCN1B, SCN2A, SCN2B, SCN8A, SHANK3, SLC13A5, SLC25A22, SLC2A1, SLC6A1, SLC6A8, SPTAN1, ST3GAL3, STRADA, STXBP1, SYNGAP1, TBC1D24, UBE3A, and WWOX, (ii) a protein having at least 90% sequence identity to (i), (iii) a functional fragment of (i) or (ii), or (iv) a transcription factor which activates expression of a gene from (i).

In some embodiments, the sequence of (i), (ii) or (iii), result in decreased expression of a protein encoded by the mRNA in liver cells at a level that is at least 2 fold, at least 5 fold, or at least 10 fold as compared to expression of the protein in liver cells from an otherwise equivalent mRNA without the sequence of (i), (ii) or (iii).

In some embodiments, the sequence of (i), (ii) or (iii), result in decreased expression of a protein encoded by the mRNA in liver cells at a level that is at least 2%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% lower than expression of the protein in liver cells from an otherwise equivalent mRNA without the sequence of (i), (ii) or (iii).

In some embodiments, the sequence of (i), (ii) or (iii), does not result in greatly decreased expression of a protein encoded by the mRNA in target cells as compared to expression of the protein in target cells from an otherwise equivalent mRNA without the sequence of (i), (ii) or (iii).

In some embodiments, the sequence of (i), (ii) or (iii), does not decrease expression of the protein encoded by the mRNA in the target cells as compared to expression of the protein in the target cells from an otherwise equivalent mRNA without the sequence of (i), (ii) or (iii).

In some embodiments, the sequence of (i), (ii) or (iii), result in expression of a protein encoded by the mRNA in target cells at a level that is at least at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of the expression of the protein in target cells from an otherwise equivalent mRNA without the sequence of (i), (ii) or (iii).

In some embodiments, the target cells are neural cells.

In some embodiments, the neural cells are cerebrum cells, brainstem cells, hippocampus cells or cerebellum cells.

In some embodiments, the neural cells are GABAergic cells.

In some embodiments, the GABAergic cells are parvalbumin expressing cells.

In some embodiments, the mRNA is expressed from a nucleic acid cassette.

In some embodiments, the nucleic acid cassette is a linear construct.

In some embodiments, the nucleic acid cassette is a vector.

In some embodiments, the vector is a plasmid.

In some embodiments, the vector is a viral vector.

In some embodiments, the viral vector is an adeno-associated virus (AAV) vector.

In some embodiments, the AAV is AAV1, AAV2, AAV3, AAV4, AAV5, AAV6, AAV7, AAV8, AAV9, or AAV-DJ.

In some embodiments, the AAV is an scAAV.

In some embodiments, the viral vector is a lentiviral vector.

In some embodiments, the method further comprises administering the vector to a subject.

In some embodiments, the method further comprises administering the mRNA to a subject.

In some embodiments, the administering comprises intraparenchymal administration, intrathecal administration, intra-cisterna magna administration, or intracerebroventricular administration.

Other features, advantages and embodiments may become apparent in view of the following description.

### BRIEF DESCRIPTION OF THE DRAWINGS

FIG. 1 is a scatter plot showing the log 2 change in brain vs liver activity for a number of test constructs.

FIG. 2 is a graph showing results of an ELISA assay of selected constructs.

FIGS. 3A-3D show representative images showing the in vivo expression of various constructs in brain and liver. FIG. 3A shows a representative image of brain expression from a mouse treated with a control vector without a detargeting element. FIG. 3B shows a representative image of liver expression from a mouse treated with a control vector without a detargeting element. FIG. 3C shows a representative image of brain expression from a mouse treated with a vector encoding an RNA containing test sequence 12 (SEQ ID NO. 12). FIG. 3D shows a representative image of liver expression from a mouse treated with a vector encoding an RNA containing test sequence 12 (SEQ ID NO. 12).

FIG. 4 is a graph showing relative expression in liver (log 2 of fold change) for several different liver detargeting elements in NHPs.

### DEFINITIONS

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The term “AAV” is an abbreviation for adeno-associated virus and may be used to refer to the virus itself or a derivative thereof. The term covers all serotypes, subtypes, and both naturally occurring and recombinant forms, except where required otherwise. The abbreviation “rAAV” refers to recombinant adeno-associated virus. The term “AAV” includes all serotypes of AAV, including AAV1, AAV2, AAV3, AAV4, AAV5, AAV6, AAV7, AAV8, AAV9, AAV9.47, AAV9(hul4), AAV10, AAV11, AAV 12, AAV13, AAVrh8, AAVrhlO, AAV-DJ, and AAV-DJ8, and hybrids thereof (i.e., chimeric AAV vectors), as well as avian AAV, bovine AAV, canine AAV, equine AAV, primate AAV, non-primate AAV, and ovine AAV. The genomic sequences of various serotypes of AAV, as well as the sequences of the native terminal repeats (TRs), Rep proteins, and capsid subunits are known in the art. Such sequences may be found in the literature or in public databases such as GenBank. A “rAAV vector” as used herein refers to an AAV vector comprising a polynucleotide sequence not of AAV origin (i.e., a polynucleotide heterologous to AAV), typically a sequence of interest for the genetic transformation of a cell. In general, the heterologous polynucleotide is flanked by at least one, and generally by two, AAV inverted terminal repeat sequences (ITRs). An rAAV vector may either be single-stranded (ssAAV) or self-complementary (scAAV). See, e.g., Raj et al., Expert Rev Hematol. 2011 October; 4(5): 539-549.An “AAV virus” or “AAV viral particle” refers to a viral particle composed of at least one AAV capsid protein and an encapsidated polynucleotide rAAV vector. If the particle comprises a heterologous polynucleotide (i.e., a polynucleotide other than a wild-type AAV genome such as a transgene to be delivered to a mammalian cell), it is typically referred to as an “rAAV viral particle” or simply an “rAAV particle”. AAVs may comprise genome components and capsids from multiple serotypes (e.g., pseudotyped vectors). For example, an AAV may comprise the genome of serotype 2 (e.g., ITRs) packaged in the capsid from serotype 5 or serotype 9. Pseudotyped vectors may demonstrate improved transduction efficiency as well as altered tropism. In some cases, an AAV serotype that can cross the blood brain barrier or infect cells of the CNS is preferred. In some aspects, the recombinant AAV vector is AAV1, AAV8, AAV9, AAVDJ, or chimeric AAV comprising features of two or more of these serotypes. In various embodiments, the AAV vector is an AAV9 vector or an scAAV9 vector. In certain embodiments, the AAV vector is an AAV9 vector or an scAAV9 vector and comprises a heterologous nucleic acid flanked by ITRs from a AAV serotype other than AAV9. In certain embodiments, the AAV vector is an AAV9 vector or an scAAV9 vector and comprises a heterologous nucleic acid flanked by AAV serotype 2 ITRs (i.e., ITR2).

The term “about” or “approximately” means within an acceptable error range for the particular value as determined by one of ordinary skill in the art, which will depend in part on how the value is measured or determined, i.e., the limitations of the measurement system. For example, “about” can mean within one or more than one standard deviation, per the practice in the art. Alternatively, “about” can mean a range of up to 20%, up to 15%, up to 10%, up to 5%, or up to 1%) of a given value.

In any of the embodiments described herein, “comprising” may be replaced with “consisting essentially of” or “consisting of.” For example, an embodiment in which a particular element is included using the open-ended term “comprising” encompasses embodiments in which the element is included using the more restrictive terms “consisting essentially of” or “consisting of”.

The terms “determining”, “measuring”, “evaluating”, “assessing”, “assaying”, “analyzing”, and their grammatical equivalents can be used interchangeably herein to refer to any form of measurement and include determining if an element is present or not (for example, detection). These terms can include both quantitative and/or qualitative determinations. Assessing may be relative or absolute.

The term “expression” refers to the process by which a nucleic acid sequence or a polynucleotide is transcribed from a DNA template (such as into mRNA or other RNA transcript) and/or the process by which a transcribed mRNA is subsequently translated into peptides, polypeptides, or proteins. Transcripts and encoded polypeptides may be collectively referred to as “gene product.” If the polynucleotide includes introns or splice sites, e.g., is derived from genomic DNA, expression may include splicing of the mRNA in a eukaryotic cell.

An “expression cassette” refers to a nucleic molecule comprising one or more regulatory elements operably linked to a coding sequence (e.g., a gene or genes) for expression.

A “transgene” refers to a portion of a nucleic acid cassette that is designed to be expressed in a cell. In some embodiments, a transgene encodes an RNA transcript, e.g., an mRNA or a functional RNA, e.g., an antisense RNA. In some embodiments, a transgene of the present disclosure encodes a therapeutic cargo, e.g., a therapeutic protein or a therapeutic RNA, and also includes one or more liver de-targeting sequences/elements to reduce expression of the transgene in liver cells.

The term “effective amount” or “therapeutically effective amount” refers to that amount of a composition described herein that is sufficient to affect the intended application, including but not limited to disease treatment, as defined below. The therapeutically effective amount may vary depending upon the intended treatment application (in a cell or in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in a target cell. The specific dose will vary depending on the particular composition chosen, the dosing regimen to be followed, whether it is administered in combination with other compounds, timing of administration, the tissue to which it is administered, and the physical delivery system in which it is carried.

A “fragment” of a nucleotide or peptide sequence is meant to refer to a sequence that is less than that believed to be the “full-length” sequence.

A “functional fragment” of a DNA, RNA, or protein sequence refers to a biologically active fragment of the sequence that is shorter than the full-length or reference DNA, RNA, or protein sequence, but which retains at least one biological activity (either functional or structural) that is substantially similar to a biological activity of the full-length or reference DNA, RNA, or protein sequence. For example, a “functional fragment” may be a fragment of a sequence disclosed herein that reduces expression of the transgene to which it is operably linked in liver cells.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell but may contain mutations. Mutant progeny that have the same function or biological activity as screened or selected for in the originally transformed cell are included herein.

The term “human derived” as used herein refers to sequences that are found in a human genome (or a human genome build), or sequences homologous thereto. A homologous sequence may be a sequence which has a region with at least 80% sequence identity (e.g., as measured by BLAST) as compared to a region of the human genome. For example, a sequence that has at least 80%, at least 85%, at least 90%, at least 91%, at least 92%, at least 93%, at least 94%, at least 95%, at least 96%, at least 97%, at least 98%, at least 99% or 100% identity to a human sequence is deemed human derived. In some cases, a regulatory element contains a human derived sequence and a non-human derived sequence such that overall the regulatory element has low sequence identity to the human genome, while a part of the regulatory element has 100% sequence identity (or local sequence identity) to a sequence in the human genome.

The term “in vitro” refers to an event that takes places outside of a subject's body. For example, an in vitro assay encompasses any assay run outside of a subject. In vitro assays encompass cell-based assays in which cells alive or dead are employed. In vitro assays also encompass a cell-free assay in which no intact cells are employed.

The term “in vivo” refers to an event that takes place in a subject's body.

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally, at a chromosomal location that is different from its natural chromosomal location, or contains only coding sequences.

As used herein, “operably linked”, “operable linkage”, “operatively linked”, or grammatical equivalents thereof refer to juxtaposition of genetic elements, e.g., a promoter, an enhancer, a polyadenylation sequence, etc., wherein the elements are in a relationship permitting them to operate in the expected manner. For instance, a regulatory element, which can comprise promoter and/or enhancer sequences, is operatively linked to a coding region if the regulatory element helps initiate transcription of the coding sequence. There may be intervening residues between the regulatory element and coding region so long as this functional relationship is maintained.

A “pharmaceutically acceptable carrier” refers to an ingredient in a pharmaceutical formulation or composition, other than an active ingredient, which is nontoxic to a subject. A pharmaceutically acceptable carrier includes, but is not limited to, a buffer, excipient, stabilizer, or preservative.

The terms “pharmaceutical formulation” or “pharmaceutical composition” refer to a preparation which is in such form as to permit the biological activity of an active ingredient contained therein to be effective, and which contains no additional components which are unacceptably toxic to a subject to which the formulation would be administered.

The term “regulatory element” refers to a nucleic acid sequence or genetic element which is capable of influencing (e.g., increasing, decreasing, or modulating) expression of an operably linked sequence, such as a gene, a coding sequence, or an RNA (e.g., an mRNA). Regulatory elements include, but are not limited to, promoter, enhancer, repressor, silencer, insulator sequences, an intron, UTR, an inverted terminal repeat (ITR) sequence, a long terminal repeat sequence (LTR), a stability element, a miRNA target site, a posttranslational response element, or a polyA sequence, or a combination thereof. Regulatory elements can function at the DNA and/or the RNA level, e.g., by modulating gene expression at the transcriptional phase, post-transcriptional phase, or at the translational phase of gene expression; by modulating the level of translation (e.g., stability elements that stabilize mRNA for translation), RNA cleavage, RNA splicing, and/or transcriptional termination; by recruiting transcriptional factors to a coding region that increase gene expression; by increasing the rate at which RNA transcripts are produced, increasing the stability of RNA produced, and/or increasing the rate of protein synthesis from RNA transcripts; and/or by preventing RNA degradation and/or increasing its stability to facilitate protein synthesis. In an exemplary embodiment, a regulatory element refers to an enhancer, repressor, promoter, or a combination thereof, particularly an enhancer plus promoter combination or a repressor plus promoter combination. In exemplary embodiments, the regulatory element is derived from a human sequence.

In general, “sequence identity” or “sequence homology”, which can be used interchangeably, refer to an exact nucleotide-to-nucleotide or amino acid-to-amino acid correspondence of two polynucleotides or polypeptide sequences, respectively. Two or more sequences (polynucleotide or amino acid) can be compared by determining their “percent identity”, also referred to as “percent homology”. The percent identity to a reference sequence (e.g., nucleic acid or amino acid sequence) may be calculated as the number of exact matches between two optimally aligned sequences divided by the length of the reference sequence and multiplied by 100. Conservative substitutions are not considered as matches when determining the number of matches for sequence identity. It will be appreciated that where the length of a first sequence (A) is not equal to the length of a second sequence (B), the percent identity of A:B sequence will be different than the percent identity of B:A sequence. Sequence alignments, such as for the purpose of assessing percent identity, may be performed by any suitable alignment algorithm or program, including but not limited to the Needleman-Wunsch algorithm, the BLAST algorithm, the Smith-Waterman algorithm (see, e.g., the EMBOSS Water aligner), and Clustal Omega alignment program (F. Sievers et al., Mol Sys Biol. 7: 539 (2011)). Optimal alignment may be assessed using any suitable parameters of a chosen algorithm, including default parameters. The BLAST program is based on the alignment method of Karlin and Altschul, Proc. Natl. Acad. Sci. USA 87:2264-2268 (1990) and as discussed in Altschul, et al., J. Mol. Biol. 215:403-410 (1990); Karlin and Altschul, Proc. Natl. Acad. Sci. USA 90:5873-5877 (1993); and Altschul et al., Nucleic Acids Res. 25:3389-3402 (1997).

The terms “subject” and “individual” are used interchangeably herein to refer to a vertebrate, preferably a mammal, more preferably a human. The methods described herein can be useful in human therapeutics, veterinary applications, and/or preclinical studies in animal models of a disease or condition.

As used herein, the terms “treat”, “treatment”, “therapy” and the like refer to obtaining a desired pharmacologic and/or physiologic effect, including, but not limited to, alleviating, delaying or slowing progression, reducing effects or symptoms, preventing onset, preventing reoccurrence, inhibiting, ameliorating onset of a diseases or disorder, obtaining a beneficial or desired result with respect to a disease, disorder, or medical condition, such as a therapeutic benefit and/or a prophylactic benefit. “Treatment,” as used herein, covers any treatment of a disease in a mammal, particularly in a human, and includes: (a) preventing the disease from occurring in a subject which may be predisposed to the disease or at risk of acquiring the disease but has not yet been diagnosed as having it; (b) inhibiting the disease, i.e., arresting its development; and (c) relieving the disease, i.e., causing regression of the disease. A therapeutic benefit includes eradication or amelioration of the underlying disorder being treated. Also, a therapeutic benefit is achieved with the eradication or amelioration of one or more of the physiological symptoms associated with the underlying disorder such that an improvement is observed in the subject, notwithstanding that the subject may still be afflicted with the underlying disorder. In some cases, for prophylactic benefit, the compositions are administered to a subject at risk of developing a particular disease, or to a subject reporting one or more of the physiological symptoms of a disease, even though a diagnosis of this disease may not have been made. The methods of the present disclosure may be used with any mammal. In some cases, the treatment can result in a decrease or cessation of symptoms. A prophylactic effect includes delaying or eliminating the appearance of a disease or condition, delaying or eliminating the onset of symptoms of a disease or condition, slowing, halting, or reversing the progression of a disease or condition, or any combination thereof.

A “variant” of a nucleotide sequence refers to a sequence having a genetic alteration or a mutation as compared to the most common wild-type DNA sequence (e.g., cDNA or a sequence referenced by its GenBank accession number) or a specified reference sequence. A variant can be shorter than the reference sequence and/or have one or more mutations relative to the reference sequence. In some cases, a variant may have a nucleotide sequence that is at least 80% identical, at least 90% identical or at least 95% identical to a reference sequence.

A “vector” as used herein refers to a nucleic acid molecule that can be used to mediate delivery of another nucleic acid molecule to which it is linked into a cell where it can be replicated or expressed. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.” Other examples of vectors include plasmids and viral vectors.

As used herein a “target cell” is generally a cell in which expression of RNA or protein product of the nucleic acid cassette is desired. A non-target cell is a cell in which expression of the RNA or protein product of the nucleic acid is not desired. As used herein “detargeting” generally refers to decreasing the expression in a non-target cell.

Unless otherwise indicated, all terms used herein have the same meaning as they would to one skilled in the art and the practice of the present invention will employ, conventional techniques of molecular biology, microbiology, and recombinant DNA technology, which are within the knowledge of those of skill of the art.

### DETAILED DESCRIPTION

Before the present invention is further described, it is to be understood that this invention is not limited to particular embodiments described, and as such may vary. It is also to be understood that the terminology used herein is for the purpose of describing particular embodiments only, and is not intended to be limiting, since the scope of the present invention will be limited only by the appended claims.

The upper and lower limits of ranges may independently be included in the ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a protein” includes a plurality of such proteins and reference to “the nucleic acid” includes reference to one or more nucleic acids and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

As summarized above, this disclosure describes a nucleic acid cassette comprising a transgene encoding an RNA, wherein the RNA comprises a sequence of (i) any of SEQ ID NOs. 1-17 or 39, (ii) a functional fragment thereof, or (iii) a sequence at least 80% identical to (i) or (ii), or any combination thereof. The transgene may encode a protein coding an mRNA, or a non-coding RNA such as a priMicroRNA, preMicroRNA, or a MicroRNA, a short non coding RNA, a long non-coding RNA, a snoRNA, a snRNA, a tRNA or an rRNA. In some cases, the nucleic acid cassette comprises a transgene encoding an mRNA, wherein the mRNA comprises a sequence of (i) any of SEQ ID NOs. 1-17 or 39, (ii) a functional fragment thereof, or (iii) a sequence at least 80% identical to (i) or (ii), or any combination thereof. These sequences reduce the expression of the transgene in liver cells relative to target cells (such as neural cells, e.g., neurons) and, as such, may be employed in a variety of gene therapy strategies that target cells that are not in the liver. Reducing expression of the transgene in liver cells relative to target cells means that the reduction in transgene expression driven by the liver de-targeting sequences disclosed herein is greater in liver cells than in the target cells. As such, while reduced transgene expression in target cells may be observed in certain embodiments, it is less than that observed in liver cells. This reduction in expression in the liver can reduce or eliminate liver toxicity in a subject receiving a gene therapy targeted to a non-liver cell or tissue, e.g., neural cells, e.g., neurons, thereby improving its safety profile.

The present disclosure further provides an RNA molecule having the sequence characteristics of an RNA encoded by any of the nucleic acid cassettes described herein. In certain embodiments, the RNA is modified to increase its stability and/or activity when administered to a subject, e.g., as a pharmaceutical composition. RNA compositions find use in a variety of therapeutic modalities delivered using a wide range of viral and non-viral delivery systems, the latter including polymeric materials, ionizable lipids, cell-penetrating and zwitterionic lipids, nanoparticles, and dendrimers (see, e.g., Kowalski et al., “Delivering the Messenger: Advances in Technologies for Therapeutic mRNA Delivery” Molecular Therapy 2019 v.27(4) pp. 710-728 and Paunovska et al. “Drug delivery systems for RNA therapeutics” Nature Reviews genetics 2022) v23 pp. 265-280).

The RNA encoded by the transgene of the nucleic acid cassette (e.g., an mRNA) may contain any combination of two, three, four or five or more of the sequences. For example, the RNA comprising a sequence of (i) any of SEQ ID NOs. 1-17 or 39, (ii) a variant or functional fragment thereof, or (iii) a sequence at least 80%, 81%, 82%, 83%, 84%, 85%, 86%, 87%, 88%, 89%, 90%, 91%, 92%, 93%, 94%, 95%, 96%, 97%, 98%, or 99% identical to (i) or (ii), may further comprise a second sequence of (i), (ii) or (iii), a third sequence of (i), (ii) or (iii), a fourth sequence of (i), (ii) or (iii), and/or five or more sequences of (i), (ii) or (iii). In any embodiment, the nucleic acid cassette may comprise two or more copies (e.g., two, three, four, five, or more than five copies) of a sequence of (i), (ii) or (iii).

In any embodiment, the sequence can be in a 3′ UTR, a 5′ UTR or an intron of a mRNA, for example. If the mRNA contains more than one of the sequences, then the sequences may be in different parts of the mRNA. In many embodiments, however, the sequences are in the 3′ UTR of the mRNA. In these embodiments, the sequence of (i) any of SEQ ID NOs. 1-17 or 39, (ii) a variant or functional fragment thereof, or (iii) a sequence at least 80%, 81%, 82%, 83%, 84%, 85%, 86%, 87%, 88%, 89%, 90%, 91%, 92%, 93%, 94%, 95%, 96%, 97%, 98%, or 99% identical to (i) or (ii), may be located in one or more of: a 3′ UTR region of the mRNA, a 5′ UTR of the mRNA or an intron of the mRNA.

Any nucleic acid described herein may be non-naturally occurring, where the term “non-naturally occurring” refers to a composition that does not exist in nature. A non-naturally occurring nucleic acid may contain: a sequence of nucleotides that is different to a nucleic acid in its natural state (i.e., having less than 100% sequence identity to a naturally occurring nucleic acid sequence). If two parts of a nucleic acid are “heterologous”, they are not part of the same nucleic acid in its natural state. For example, in some embodiments, a nucleic acid cassette may be composed of a promoter, a coding sequence and a terminator, where the promoter, coding sequence and terminator are in operable linkage. In these embodiments, the promoter may be heterologous to the coding sequence, meaning that the promoter does not drive the expression of that coding sequence in a wild type cell. In any embodiment, the nucleic acid cassette may additionally comprise an enhancer.

In any embodiment, the RNA encoded by the transgene of the nucleic acid cassette may comprise a functional fragment of at least 10, at least 15, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, at least 200, at least 210, or at least 220 contiguous nucleotides of any of SEQ ID NOS. 1-17 or 39, which may or may not contain 1, 2, 3, 4, 5, 6, 7, 8, 9, 10 or more than 10 mismatches relative to SEQ ID NOS. 1-17 or 39, for example. Such a fragment may start anywhere in SEQ ID NOS. 1-17 or 39, e.g., at position 1, 21, 41, 61, 81, 101 or 121, for example.

In certain embodiments, a functional fragment comprises any contiguous stretch of nucleotides in SEQ ID NOS: 1, 5, 7, or 10 of at least 10, at least 11, at least 12, at least 13, at least 14, at least 15, at least 16, at least 17, at least 18, at least 19, at least 20, or at least 21 nucleotides in length. In certain embodiments, a functional fragment of SEQ ID NO: 1, 5, 7, or 10 comprises one, two, three, or four mismatches as compared to the corresponding contiguous stretch of nucleotides in SEQ ID NO: 1, 5, 7, or 10. A functional fragment may start at any nucleotide in SEQ ID NO: 1, 5, 7, or 10 that allows for its full representation in SEQ ID NO: 1, 5, 7, or 10. Thus, a functional fragment of 10 contiguous nucleotides can start at any one of nucleotides 1 to 13 of SEQ ID NO: 1, 5, 7, or 10, a functional fragment of 11 contiguous nucleotides can start at any one of nucleotides 1 to 12 of SEQ ID NO: 1, 5, 7, or 10, a functional fragment of 12 contiguous nucleotides can start at any one of nucleotides 1 to 11 of SEQ ID NO: 1, 5, 7, or 10, a functional fragment of 13 contiguous nucleotides can start at any one of nucleotides 1 to 10 of SEQ ID NO: 1, 5, 7, or 10, a functional fragment of 14 contiguous nucleotides can start at any one of nucleotides 1 to 9 of SEQ ID NO: 1, 5, 7, or 10, a functional fragment of 15 contiguous nucleotides can start at any one of nucleotides 1 to 8 of SEQ ID NO: 1, 5, 7, or 10, a functional fragment of 16 contiguous nucleotides can start at any one of nucleotides 1 to 7 of SEQ ID NO: 1, 5, 7, or 10, a functional fragment of 17 contiguous nucleotides can start at any one of nucleotides 1 to 6 of SEQ ID NO: 1, 5, 7, or 10, a functional fragment of 18 contiguous nucleotides can start at any one of nucleotides 1 to 5 of SEQ ID NO: 1, 5, 7, or 10, a functional fragment of 19 contiguous nucleotides can start at any one of nucleotides 1 to 4 of SEQ ID NO: 1, 5, 7, or 10, a functional fragment of 20 contiguous nucleotides can start at any one of nucleotides 1 to 3 of SEQ ID NO: 1, 5, 7, or 10, and a functional fragment of 21 contiguous nucleotides can start at nucleotides 1 or 2 of SEQ ID NO: 1, 5, 7, or 10.

In certain embodiments, a functional fragment comprises any contiguous stretch of nucleotides in SEQ ID NO: 2 of at least 10, at least 11, at least 12, at least 13, at least 14, at least 15, at least 16, at least 17, or at least 18, nucleotides in length. In certain embodiments, a functional fragment of SEQ ID NO: 2 comprises one, two, three, or four mismatches as compared to the corresponding contiguous stretch of nucleotides in SEQ ID NO: 2. A functional fragment may start at any nucleotide in SEQ ID NO: 2 that allows for its full representation in SEQ ID NO: 2. Thus, a functional fragment of 10 contiguous nucleotides can start at any one of nucleotides 1 to 10 of SEQ ID NO: 2, a functional fragment of 11 contiguous nucleotides can start at any one of nucleotides 1 to 9 of SEQ ID NO: 2, a functional fragment of 12 contiguous nucleotides can start at any one of nucleotides 1 to 8 of SEQ ID NO: 2, a functional fragment of 13 contiguous nucleotides can start at any one of nucleotides 1 to 7 of SEQ ID NO: 2, a functional fragment of 14 contiguous nucleotides can start at any one of nucleotides 1 to 6 of SEQ ID NO: 2, a functional fragment of 15 contiguous nucleotides can start at any one of nucleotides 1 to 5 of SEQ ID NO: 2, a functional fragment of 16 contiguous nucleotides can start at any one of nucleotides 1 to 4 of SEQ ID NO: 2, a functional fragment of 17 contiguous nucleotides can start at any one of nucleotides 1 to 3 of SEQ ID NO: 2, a functional fragment of 18 contiguous nucleotides can start at nucleotides 1 or 2 of SEQ ID NO: 2.

In certain embodiments, a functional fragment comprises any contiguous stretch of nucleotides in SEQ ID NOS: 3 or 8 of at least 10, at least 11, at least 12, at least 13, at least 14, at least 15, at least 16, at least 17, at least 18, at least 19, or at least 20 nucleotides in length. In certain embodiments, a functional fragment of SEQ ID NO: 3 or 8 comprises one, two, three, or four mismatches as compared to the corresponding contiguous stretch of nucleotides in SEQ ID NO: 3 or 8. A functional fragment may start at any nucleotide in SEQ ID NO: 3 or 8 that allows for its full representation in SEQ ID NO: 3 or 8. Thus, a functional fragment of 10 contiguous nucleotides can start at any one of nucleotides 1 to 12 of SEQ ID NO: 3 or 8, a functional fragment of 11 contiguous nucleotides can start at any one of nucleotides 1 to 11 of SEQ ID NO: 3 or 8, a functional fragment of 12 contiguous nucleotides can start at any one of nucleotides 1 to 10 of SEQ ID NO: 3 or 8, a functional fragment of 13 contiguous nucleotides can start at any one of nucleotides 1 to 9 of SEQ ID NO: 3 or 8, a functional fragment of 14 contiguous nucleotides can start at any one of nucleotides 1 to 8 of SEQ ID NO: 3 or 8, a functional fragment of 15 contiguous nucleotides can start at any one of nucleotides 1 to 7 of SEQ ID NO: 3 or 8, a functional fragment of 16 contiguous nucleotides can start at any one of nucleotides 1 to 6 of SEQ ID NO: 3 or 8, a functional fragment of 17 contiguous nucleotides can start at any one of nucleotides 1 to 5 of SEQ ID NO: 3 or 8, a functional fragment of 18 contiguous nucleotides can start at any one of nucleotides 1 to 4 of SEQ ID NO: 3 or 8, a functional fragment of 19 contiguous nucleotides can start at any one of nucleotides 1 to 3 of SEQ ID NO: 3 or 8, a functional fragment of 20 contiguous nucleotides can start at nucleotides or 2 of SEQ ID NO: 3 or 8.

In certain embodiments, a functional fragment comprises any contiguous stretch of nucleotides in SEQ ID NOs: 4 or 39 of at least 10, at least 11, at least 12, at least 13, at least 14, at least 15, at least 16, at least 17, at least 18, at least 19, at least 20, at least 21, or at least 22 nucleotides in length. In certain embodiments, a functional fragment of SEQ ID NOs: 4 or 39 comprises one, two, three, or four mismatches as compared to the corresponding contiguous stretch of nucleotides in SEQ ID NOs: 4 or 39. A functional fragment may start at any nucleotide in SEQ ID NOs: 4 or 39 that allows for its full representation in SEQ ID NOs: 4 or 39. Thus, a functional fragment of 10 contiguous nucleotides can start at any one of nucleotides 1 to 14 of SEQ ID NOs: 4 or 39, a functional fragment of 11 contiguous nucleotides can start at any one of nucleotides 1 to 13 of SEQ ID NOs: 4 or 39, a functional fragment of 12 contiguous nucleotides can start at any one of nucleotides 1 to 12 of SEQ ID NOs: 4 or 39, a functional fragment of 13 contiguous nucleotides can start at any one of nucleotides 1 to 11 of SEQ ID NOs: 4 or 39, a functional fragment of 14 contiguous nucleotides can start at any one of nucleotides 1 to 10 of SEQ ID NOs: 4 or 39, a functional fragment of 15 contiguous nucleotides can start at any one of nucleotides 1 to 9 of SEQ ID NOs: 4 or 39, a functional fragment of 16 contiguous nucleotides can start at any one of nucleotides 1 to 8 of SEQ ID NOs: 4 or 39, a functional fragment of 17 contiguous nucleotides can start at any one of nucleotides 1 to 7 of SEQ ID NOs: 4 or 39, a functional fragment of 18 contiguous nucleotides can start at any one of nucleotides 1 to 6 of SEQ ID NOs: 4 or 39, a functional fragment of 19 contiguous nucleotides can start at any one of nucleotides 1 to 5 of SEQ ID NOs: 4 or 39, a functional fragment of 20 contiguous nucleotides can start at any one of nucleotides 1 to 4 of SEQ ID NOs: 4 or 39, a functional fragment of 21 contiguous nucleotides can start at any one of nucleotides 1 to 3 of SEQ ID NOs: 4 or 39, and a functional fragment of 22 contiguous nucleotides can start at nucleotides 1 or 2 of SEQ ID NOs: 4 or 39.

In certain embodiments, a functional fragment comprises any contiguous stretch of nucleotides in SEQ ID NOS: 6, 9 or 11 of at least 10, at least 11, at least 12, at least 13, at least 14, at least 15, at least 16, at least 17, at least 18, or at least 19 nucleotides in length. In certain embodiments, a functional fragment of SEQ ID NO: 6, 9 or 11 comprises one, two, three, or four mismatches as compared to the corresponding contiguous stretch of nucleotides in SEQ ID NO: 6, 9 or 11. A functional fragment may start at any nucleotide in SEQ ID NO: 6, 9 or 11 that allows for its full representation in SEQ ID NO: 6, 9 or 11. Thus, a functional fragment of 10 contiguous nucleotides can start at any one of nucleotides 1 to 11 of SEQ ID NO: 6, 9 or 11, a functional fragment of 11 contiguous nucleotides can start at any one of nucleotides 1 to 10 of SEQ ID NO: 6, 9 or 11, a functional fragment of 12 contiguous nucleotides can start at any one of nucleotides 1 to 9 of SEQ ID NO: 6, 9 or 11, a functional fragment of 13 contiguous nucleotides can start at any one of nucleotides 1 to 8 of SEQ ID NO: 6, 9 or 11, a functional fragment of 14 contiguous nucleotides can start at any one of nucleotides 1 to 7 of SEQ ID NO: 6, 9 or 11, a functional fragment of 15 contiguous nucleotides can start at any one of nucleotides 1 to 6 of SEQ ID NO: 6, 9 or 11, a functional fragment of 16 contiguous nucleotides can start at any one of nucleotides 1 to 5 of SEQ ID NO: 6, 9 or 11, a functional fragment of 17 contiguous nucleotides can start at any one of nucleotides 1 to 4 of SEQ ID NO: 6, 9 or 11, a functional fragment of 18 contiguous nucleotides can start at any one of nucleotides 1 to 3 of SEQ ID NO: 6, 9 or 11, a functional fragment of 19 contiguous nucleotides can start at nucleotides 1 or 2 of SEQ ID NO: 6, 9 or 11.

In certain embodiments, a functional fragment comprises any contiguous stretch of nucleotides in SEQ ID NOs: 12-17 of at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, at least 101, at least 102, at least 103, at least 104, at least 105, at least 106, at least 107, at least 108, at least 109, at least 110, at least 111, at least 112, at least 113, at least 114, at least 115, at least 116, at least 117, at least 118, at least 119, at least 120, at least 121, at least 122, at least 123, at least 124, at least 125, at least 126, at least 127, at least 128, at least 129, at least 130, at least 131, at least 132, at least 133, at least 134, at least 135, at least 136, at least 137, at least 138, at least 139, at least 140, at least 141, at least 142, at least 143, at least 144, at least 145, at least 146, at least 147, at least 148, at least 149, at least 150, or at least 151 nucleotides in length. In certain embodiments, a functional fragment comprises any contiguous stretch of nucleotides in SEQ ID NOs: 12-15 or 17 of at least 152, at least 153, at least 154, at least 155, at least 156, at least 157, at least 158, at least 159, at least 160, at least 161, at least 162, at least 163, at least 164, at least 165, at least 166, at least 167, at least 168, at least 169, at least 170, at least 171, at least 172, at least 173, at least 174, at least 175, or at least 176 nucleotides in length. In certain embodiments, a functional fragment comprises any contiguous stretch of nucleotides in SEQ ID NOs: 13, 14 or 17 of at least 177, at least 178, or at least 179 nucleotides in length. In certain embodiments, a functional fragment comprises any contiguous stretch of nucleotides in SEQ ID NOs: 14 or 17 of at least 180, at least 181, at least 182, at least 183, at least 184, at least 185, at least 186, at least 187, at least 188, at least 189, at least 190, at least 191, at least 192, at least 193, at least 194, at least 195, at least 196, at least 197, at least 198, at least 199, at least 200, at least 201, at least 202, at least 203, at least 204, at least 205, at least 206, at least 207, at least 208, at least 209, at least 210, at least 211, at least 212, at least 213, at least 214, at least 215, at least 216, at least 217, at least 218, at least 219, at least 220, at least 221, at least 222, at least 223, at least 224, at least 225, or at least 226 nucleotides in length. In certain embodiments, a functional fragment comprises any contiguous stretch of nucleotides in SEQ ID NO: 14 of 227 nucleotides in length. In certain embodiments, a functional fragment of any one of SEQ ID NOs: 12-17 comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 mismatches as compared to the corresponding contiguous stretch of nucleotides in SEQ ID NOs: 12-17. A functional fragment may start at any nucleotide in SEQ ID NO: 12-17 that allows for its full representation in SEQ ID NO: 12-17.

In any embodiment, the RNA may comprise one or more binding sites for a miRNA. In these embodiments, the RNA may comprise 6, 7, 8, 9 or 10 contiguous nucleotides at the 3′ end of any of SEQ ID NOS: 1-11, which potentially base pair the seed region of a miRNA such as miR-22-3p, miR-1258-5p, miR-5589-3p, miR-17-5p, miR-203a, miR-122-3p, miR-93-5p, or miR-122-5p (which is at the 5′ end of those miRNAs). In some embodiments, the RNA may comprise a miRNA binding site for a miRNA selected from miR-22-3p, miR-1258-5p, miR-5589-3p, miR-17-5p, miR-203a, miR-122-3p, miR-93-5p, miR-122-5p or a complement thereof. In some embodiments, the RNA may comprise a binding site for a miRNA produced from a mir-22, mir-1258, mir-5589, mir-17, mir-203a, mir-93, or mir-122 gene. The one or more binding sites for a miRNA may comprise any of SEQ ID NOS: 1-11 or 39. In some embodiments, the sequence may be identical to SEQ ID NOS: 1-11 or 39 except that it has one, two, three of four mismatches relative to SEQ ID NOS: 1-11 or 39, for example.

As would be apparent, the nucleic acid cassette itself (which is DNA) may contain (i) any of SEQ ID NOs 18-34 or 40, (ii) a variant, a functional fragment, or a combination thereof, or (iii) a sequence at least 80%, 81%, 82%, 83%, 84%, 85%, 86%, 87%, 88%, 89%, 90%, 91%, 92%, 93%, 94%, 95%, 96%, 97%, 98%, or 99% identical to (i) or (ii), wherein inclusion of the sequence reduces the expression of the protein or RNA encoded by the cassette in liver cells in the organism, relative to a target tissue, e.g., a neuronal tissue, e.g., a neuronal cell in the brain.

In some embodiments, the mRNA may encode a polypeptide, e.g., a therapeutic protein, which protein may be intracellular, membrane bound or secreted, for example.

A transgene encoding an mRNA encoding a therapeutic protein is sometimes referred to herein as a therapeutic transgene. In some embodiments, the therapeutic protein one that is associated with a neural disease or disorder, e.g., a protein whose aberrant function (e.g., resulting from a genetic mutation or abnormality) is associated with a neural disease or disorder.

Neural diseases and disorders include those associated with one or more genetic mutations as well as those with unknown etiologies. In some embodiments, neural diseases and disorders include conditions associated with epileptic seizures, neurodegenerative disorders, and/or neurodevelopmental disorders. Examples of neural diseases or disorders include, but are not limited to: Alpers-Huttenlocher Syndrome, Angelman Syndrome, CDKL5 Deficiency Disorder, Dravet Syndrome, Rett Syndrome, Parkinson's Disease and Parkinson's LIDS (side effect of Parkinson's medication), Alzheimer's disease, Creatine Transporter Deficiency, FOXG1 Syndrome, Fragile X Syndrome, Phelan-McDermid Syndrome, Childhood Absence Epilepsy, Childhood Epilepsy Centrotemporal Spikes (Benign Rolandic Epilepsy), Dravet Syndrome, Early Myoclonic Encephalopathy (EME), Epilepsy Eyelid Myoclonia Jeavons Syndrome, Epilepsy of Infancy with Migrating Focal Seizures, Epilepsy Myoclonic Absences, Epileptic Encephalopathy Continuous Spike and Wave During Sleep CSWS, Infantile Spasms (West Syndrome), Juvenile Myoclonic Epilepsy, Landau-Kleffner Syndrome, Lennox-Gastaut Syndrome (LGS), Myoclonic Epilepsy in Infancy, Ohtahara Syndrome, Panayiotopoulos Syndrome, Progressive Myoclonic Epilepsies, Reflex Epilepsies, Self-Limited Familial and Non-Familial Neonatal Infantile Seizures, Self-Limited Late Onset Occipital Epilepsy Gastaut Syndrome, Epilepsy Generalized Tonic Clonic Seizures Alone, Genetic Epilepsy with Febrile Seizures Plus, Juvenile Absence Epilepsy, Myoclonic Atonic Epilepsy Doose Syndrome, Sleep-related Hypermotor Epilepsy (SHE), febrile seizures, focal epilepsy, West Syndrome, Early Onset Epilepsy, Benign Familial Infantile Epilepsy, and Attention Deficit-Hyperactivity Disorder.

A number of genetic abnormalities have been associated with epilepsies, including many of the forgoing neural diseases and disorders. Examples of genes affected by these genetic abnormalities, i.e., genes whose activity and/or expression has been altered by genetic mutation(s), include: ALDH7A1, ARHGEF9, ARX, BRAT1, CACNA1A, CACNA1D, CACNB4, CDKL5, CHD2, CHRNA2, CHRNA4, CHRNB2, CLCN2, CLN, CLN2, DEPDC5, DNM1, FGF13, FMR1, FOLR1, FOXG1, GABRA1, GABRB3, GABRD, GABRG2, GBA1, GRIN2A, GRIN2B, GRN, HCN1, HCN4, KCNQ2, KCNQ3, KCNT1, KV3.1, KV3.2, KV3.3, LGI1, MECP2, MEF2C, Myoclonin1/EFHC1, NPRL2, PCDH19, PLCB1, PNKP, POLG1, PRRT2, PTEN, SCN1A, SCN1B, SCN2A, SCN2B, SCN8A, SHANK3, SLC13A5, SLC25A22, SLC2A1, SLC6A1, SLC6A8, SPTAN1, ST3GAL3, STRADA, STXBP1, SYNGAP1, TBC1D24, UBE3A, and WWOX.

Thus, in embodiments in which the mRNA encodes a therapeutic protein for treatment of a neural disease or disorder, the therapeutic protein may be (i) a functional form of a protein encoded by a gene selected from: ALDH7A1, ARHGEF9, ARX, BRAT1, CACNA1A, CACNA1D, CACNB4, CDKL5, CHD2, CHRNA2, CHRNA4, CHRNB2, CLCN2, CLN, CLN2, DEPDC5, DNM1, FGF13, FMR1, FOLR1, FOXG1, GABRA1, GABRB3, GABRD, GABRG2, GBA1, GRIN2A, GRIN2B, GRN, HCN1, HCN4, KCNQ2, KCNQ3, KCNT1, KV3.1, KV3.2, KV3.3, LGI1, MECP2, MEF2C, Myoclonin1/EFHC1, NPRL2, PCDH19, PLCB1, PNKP, POLG1, PRRT2, PTEN, SCN1A, SCN1B, SCN2A, SCN2B, SCN8A, SHANK3, SLC13A5, SLC25A22, SLC2A1, SLC6A1, SLC6A8, SPTAN1, ST3GAL3, STRADA, STXBP1, SYNGAP1, TBC1D24, UBE3A, and WWOX, (ii) a protein having at least 80%, 81%, 82%, 83%, 84%, 85%, 86%, 87%, 88%, 89%, 90%, 91%, 92%, 93%, 94%, 95%, 96%, 97%, 98%, or 99% sequence identity to (i), (iii) a variant or functional fragment of (i) or (ii), or (iv) a transcription factor which activates expression of a gene from (i). A transcription factor encoded by the mRNA may be an engineered transcription factor or a naturally occurring transcription factor.

In some embodiments, the sequence of (i) any of SEQ ID NOs. 1-17 or 39, (ii) a variant, functional fragment, or combination thereof, or (iii) a sequence at least 80%, 81%, 82%, 83%, 84%, 85%, 86%, 87%, 88%, 89%, 90%, 91%, 92%, 93%, 94%, 95%, 96%, 97%, 98%, or 99% identical to (i) or (ii), may result in decreased expression of a polypeptide encoded by the mRNA in liver cells as compared to expression of the polypeptide in liver cells from an otherwise equivalent mRNA without the sequence of (i), (ii), or (iii). For example, an mRNA containing a sequence of (i), (ii), or (iii), may result in decreased expression of a polypeptide encoded by the mRNA in liver cells at a level that is at least 1.5 fold, at least 2-fold, at least 5-fold, or at least 10-fold as compared to expression of the polypeptide in liver cells from an otherwise equivalent mRNA without the sequence of (i), (ii), or (iii). In these embodiments, the reduction of expression of the polypeptide in liver cells is greater than the reduction of expression of the polypeptide in the target cells when compared to otherwise equivalent mRNA without the sequence of (i), (ii), or (iii).

In some embodiments, the sequence of (i) any of SEQ ID NOs. 1-17 or 39, (ii) a variant, functional fragment, or combination thereof, or (iii) a sequence at least 80%, 81%, 82%, 83%, 84%, 85%, 86%, 87%, 88%, 89%, 90%, 91%, 92%, 93%, 94%, 95%, 96%, 97%, 98%, or 99% identical to (i) or (ii), may result in decreased expression of a polypeptide encoded by the mRNA in liver cells at a level that is at least 2%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% lower than expression of the polypeptide in liver cells from an otherwise equivalent mRNA without the sequence of (i), (ii) or (iii). In these embodiments, the reduction of expression of the polypeptide in liver cells is greater than the reduction of expression of the polypeptide in the target cells when compared to otherwise equivalent mRNA without the sequence of (i), (ii), or (iii).

In some embodiments, the sequence of (i) any of SEQ ID NOs. 1-17 or 39, (ii) a variant, functional fragment, or combination thereof, or (iii) a sequence at least 80%, 81%, 82%, 83%, 84%, 85%, 86%, 87%, 88%, 89%, 90%, 91%, 92%, 93%, 94%, 95%, 96%, 97%, 98%, or 99% identical to (i) or (ii), does not result in significantly decreased expression of a polypeptide encoded by the mRNA in target cells as compared to expression of the polypeptide in target cells from an otherwise equivalent mRNA without the sequence of (i), (ii) or (iii). In some embodiments, the sequence of (i), (ii) or (iii), may result in expression of a polypeptide encoded by the mRNA in target cells at a level that is at least at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of the expression of the polypeptide in target cells from an otherwise equivalent mRNA without the sequence of (i), (ii) or (iii). In these embodiments, the reduction of expression of the polypeptide in liver cells is greater than the reduction of expression of the polypeptide in the target cells when compared to otherwise equivalent mRNA without the sequence of (i), (ii), or (iii).

In some cases, the target cells may be neural cells, muscle cells, cardiac cells, skin cells, immune cells, hematopoetic cells, cancer cells, pancreatic cells or kidney cells. In any of these embodiments, the target cells may be neural cells, e.g., cerebrum cells, brainstem cells, hippocampus cells or cerebellum cells. For example, in these embodiments, the neural cells may be GABAergic cells, e.g., parvalbumin expressing cells. In some cases, the target cell may be a CNS cell, such as an excitatory neuron, a dopaminergic neuron, a glial cell, an ependymal cell, an oligodendrocyte, an astrocyte, a microglia, a motor neuron, a vascular cell, a GABAergic neuron, or a non-GABAergic neuron (e.g., a cell that does not express one or more of GAD2, GAD1, NKX2.1, DLX1, DLX5, SST and VIP), a non-PV neuron (e.g., a GABAergic neuron that does not express parvalbumin), or another CNS cell (e.g., a CNS cell type that have never expressed any of PV, GAD2, GAD1, NKX2.1, DLX1, DLX5, SST and VIP).

The cassette may be linear, circular and, in some embodiments, the nucleic acid cassette may be a vector such as a plasmid or viral vector, e.g., an adeno-associated virus (AAV) vector or lentiviral vector. In particular embodiments, the viral vector may be an AAV vector selected from AAV1, AAV2, AAV3, AAV4, AAV5, AAV6, AAV7, AAV8, AAV9, AAV9.47, AAV9(hul4), AAV10, AAV11, AAV 12, AAV13, AAVrh8, AAVrhlO, AAV-DJ, and AAV-DJ8, and hybrids thereof.

Also provided is a nucleic acid cassette comprising a transgene encoding an RNA, wherein the RNA comprises a miRNA binding site for a miRNA selected from miR-22-3p, miR-1258-5p, miR-5589-3p, miR-17-5p, miR-203a, miR-122-3p, miR-93-5p, miR-122-5p, or a complement thereof. In some embodiments, the RNA may comprise a binding site for a miRNA produced from a mir-22, mir-1258, mir-5589, mir-17 or mir-203a, mir-93, or mir-122 gene. The miRNA binding site should not be in the naturally occurring version of the RNA, if the RNA is otherwise naturally occurring. In some embodiments, the cassette may comprise two or more, three or more or four or more binding sites for miRNAs selected from miR-22-3p, miR-1258-5p, miR-5589-3p, miR-17-5p, miR-203a, miR-122-3p, miR-93-5p, miR-122-5p, or a complement thereof, for example. In some embodiments, the RNA is a mRNA, e.g., an mRNA encoding a therapeutic protein (as described elsewhere herein). In these embodiments, the binding sites may be anywhere in the mRNA, particularly in a non-coding sequence such as a 3′ UTR region, a 5′ UTR, an intron, or any combination thereof.

In any embodiment, the nucleic acid cassette may be non-naturally occurring, meaning that, for example, the miRNA binding site may be heterologous to the mRNA. In any embodiment, the nucleic acid cassette may comprise a promoter and/or enhancer. In some embodiments, this nucleic acid cassette may be composed of a promoter, a coding sequence and a terminator, where the promoter, coding sequence and terminator are in operable linkage. In these embodiments, the promoter may be heterologous to the coding sequence, meaning that the promoter does not drive the expression of that coding sequence in a wild type cell. In any embodiment, the nucleic acid cassette may additionally comprise an enhancer.

In some embodiments, the mRNA may encode a polypeptide, e.g., a therapeutic protein, which protein may be intracellular, membrane bound or secreted, for example.

In some embodiments, the protein is a protein associated with a neural disease or disorder.

Neural diseases and disorders include those associated with one or more genetic mutations as well as those with unknown etiologies. In broad terms, neural diseases and disorders include conditions associated with epileptic seizures, neurodegenerative disorders, and/or neurodevelopmental disorders. Examples of neural diseases or disorders include, but are not limited to: Alpers-Huttenlocher Syndrome, Angelman Syndrome, CDKL5 Deficiency Disorder, Dravet Syndrome, Rett Syndrome, Parkinson's Disease and Parkinson's LIDS (side effect of Parkinson's medication), Alzheimer's disease, Creatine Transporter Deficiency, FOXG1 Syndrome, Fragile X Syndrome, Phelan-McDermid Syndrome, Childhood Absence Epilepsy, Childhood Epilepsy Centrotemporal Spikes (Benign Rolandic Epilepsy), Dravet Syndrome, Early Myoclonic Encephalopathy (EME), Epilepsy Eyelid Myoclonia Jeavons Syndrome, Epilepsy of Infancy with Migrating Focal Seizures, Epilepsy Myoclonic Absences, Epileptic Encephalopathy Continuous Spike and Wave During Sleep CSWS, Infantile Spasms (West Syndrome), Juvenile Myoclonic Epilepsy, Landau-Kleffner Syndrome, Lennox-Gastaut Syndrome (LGS), Myoclonic Epilepsy in Infancy, Ohtahara Syndrome, Panayiotopoulos Syndrome, Progressive Myoclonic Epilepsies, Reflex Epilepsies, Self-Limited Familial and Non-Familial Neonatal Infantile Seizures, Self-Limited Late Onset Occipital Epilepsy Gastaut Syndrome, Epilepsy Generalized Tonic Clonic Seizures Alone, Genetic Epilepsy with Febrile Seizures Plus, Juvenile Absence Epilepsy, Myoclonic Atonic Epilepsy Doose Syndrome, Sleep-related Hypermotor Epilepsy (SHE), febrile seizures, focal epilepsy, West Syndrome, Early Onset Epilepsy, Benign Familial Infantile Epilepsy, and Attention Deficit-Hyperactivity Disorder.

A number of genetic abnormalities have been associated with epilepsies, including many of the forgoing neural diseases and disorders. Examples of genes affected by these genetic abnormalities, i.e., genes whose activity and/or expression has been altered by genetic mutation(s), include: ALDH7A1, ARHGEF9, ARX, BRAT1, CACNA1A, CACNA1D, CACNB4, CDKL5, CHD2, CHRNA2, CHRNA4, CHRNB2, CLCN2, CLN, CLN2, DEPDC5, DNM1, FGF13, FMR1, FOLR1, FOXG1, GABRA1, GABRB3, GABRD, GABRG2, GBA1, GRIN2A, GRIN2B, GRN, HCN1, HCN4, KCNQ2, KCNQ3, KCNT1, KV3.1, KV3.2, KV3.3, LGI1, MECP2, MEF2C, Myoclonin1/EFHC1, NPRL2, PCDH19, PLCB1, PNKP, POLG1, PRRT2, PTEN, SCN1A, SCN1B, SCN2A, SCN2B, SCN8A, SHANK3, SLC13A5, SLC25A22, SLC2A1, SLC6A1, SLC6A8, SPTAN1, ST3GAL3, STRADA, STXBP1, SYNGAP1, TBC1D24, UBE3A, and WWOX.

Thus, in embodiments in which the mRNA encodes a therapeutic protein for treatment of a neural disease or disorder, the therapeutic protein may be (i): a protein encoded by a gene selected from: ALDH7A1, ARHGEF9, ARX, BRAT1, CACNA1A, CACNA1D, CACNB4, CDKL5, CHD2, CHRNA2, CHRNA4, CHRNB2, CLCN2, CLN, CLN2, DEPDC5, DNM1, FGF13, FMR1, FOLR1, FOXG1, GABRA1, GABRB3, GABRD, GABRG2, GBA1, GRIN2A, GRIN2B, GRN, HCN1, HCN4, KCNQ2, KCNQ3, KCNT1, KV3.1, KV3.2, KV3.3, LGI1, MECP2, MEF2C, Myoclonin1/EFHC1, NPRL2, PCDH19, PLCB1, PNKP, POLG1, PRRT2, PTEN, SCN1A, SCN1B, SCN2A, SCN2B, SCN8A, SHANK3, SLC13A5, SLC25A22, SLC2A1, SLC6A1, SLC6A8, SPTAN1, ST3GAL3, STRADA, STXBP1, SYNGAP1, TBC1D24, UBE3A, and WWOX, (ii) a protein having at least 90% sequence identity to (i), (iii) a functional fragment of (i) or (ii), or (iv) a transcription factor which activates expression of a gene from (i). A transcription factor encoded by the mRNA may be an engineered transcription factor or a naturally occurring transcription factor. In some embodiments, the sequence of (i) any of SEQ ID NOs. 1-17 or 39, (ii) a functional fragment thereof, or (iii) a sequence at least 80% identical to (i) or (ii), may result in decreased expression of a polypeptide encoded by the mRNA in liver cells as compared to expression of the polypeptide in liver cells from an otherwise equivalent mRNA without the sequence of (i), (ii) or (iii). For example, an mRNA containing a sequence of (i), (ii) or (iii), may result in decreased expression of a polypeptide encoded by the mRNA in liver cells at a level that is at least 2 fold, at least 5 fold, or at least 10 fold as compared to expression of the polypeptide in liver cells from an otherwise equivalent mRNA without the sequence of (i), (ii) or (iii).

In some embodiments, the sequence of (i) any of SEQ ID NOs. 1-17 or 39, (ii) a functional fragment thereof, or (iii) a sequence at least 80% identical to (i) or (ii), may result in decreased expression of a polypeptide encoded by the mRNA in liver cells at a level that is at least 2%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% lower than expression of the polypeptide in liver cells from an otherwise equivalent mRNA without the sequence of (i), (ii) or (iii).

In some embodiments, the sequence of (i) any of SEQ ID NOs. 1-17 or 39, (ii) a functional fragment thereof, or (iii) a sequence at least 80% identical to (i) or (ii), does not result in decreased expression of a polypeptide encoded by the mRNA in target cells as compared to expression of the polypeptide in target cells from an otherwise equivalent mRNA without the sequence of (i), (ii) or (iii). In some embodiments, the sequence of (i), (ii) or (iii), does not decrease expression of the polypeptide encoded by the mRNA in the target cells as compared to expression of the polypeptide in the target cells from an otherwise equivalent mRNA without the sequence of (i), (ii) or (iii). In these embodiments, the sequence of (i), (ii) or (iii), may result in expression of a polypeptide encoded by the mRNA in target cells at a level that is at least at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of the expression of the polypeptide in target cells from an otherwise equivalent mRNA without the sequence of (i), (ii) or (iii). In any of these embodiments, the target cells may be neural cells, e.g., cerebrum cells, brainstem cells, hippocampus cells or cerebellum cells. For example, in these embodiments, the neural cells may be GABAergic cells, e.g., parvalbumin expressing cells.

The cassette may be linear, circular and, in some embodiments, the nucleic acid cassette may be a vector such as a plasmid or viral vector, e.g., an adeno-associated virus (AAV) vector or lentiviral vector. In particular embodiments, the viral vector may be AAV vector selected from is AAV1, AAV2, AAV3, AAV4, AAV5, AAV6, AAV7, AAV8, AAV9, AAV-DJ and scAAV.

Also provided is an mRNA encoded by a nucleic acid cassette described above.

Also provided is a method of decreasing liver expression of a polypeptide encoded by an mRNA relative to expression of the polypeptide in a target tissue. In these embodiments, the method may comprise constructing a nucleic acid cassette to include a liver de-targeting sequence as described herein in an RNA encoded therein. For example, the nucleic acid cassette can be constructed to include a sequence of (i) one of SEQ ID NOs 1-17 or 39, (ii) a variant, functional fragment, or a combination thereof, or (iii) a sequence at least 80%, 81%, 82%, 83%, 84%, 85%, 86%, 87%, 88%, 89%, 90%, 91%, 92%, 93%, 94%, 95%, 96%, 97%, 98%, or 99% identical to (i) or (ii) in the mRNA encoded therein. Details of the cassettes made by this method are described herein. In some embodiments, the method may comprise introducing an expression cassette as described herein or an mRNA encoded thereby into an organism, e.g., a human subject, wherein inclusion of any one or more of the liver detargeting sequences reduces the expression of the protein in liver cells in the organism, relative to a target tissue.

In some embodiments, the sequence of (i) any of SEQ ID NOs. 1-17 or 39, (ii) a functional fragment thereof, or (iii) a sequence at least 80% identical to (i) or (ii), result in decreased expression of a polypeptide encoded by the mRNA in liver cells at a level that is at least 2 fold, at least 5 fold, or at least 10 fold as compared to expression of the polypeptide in liver cells from an otherwise equivalent mRNA without the sequence of (i), (ii) or (iii). In these embodiments, the sequence of (i), (ii) or (iii), may result in decreased expression of a polypeptide encoded by the mRNA in liver cells at a level that is at least 2%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% lower than expression of the polypeptide in liver cells from an otherwise equivalent mRNA without the sequence of (i), (ii) or (iii).

In some embodiments, the sequence of (i) any of SEQ ID NOs. 1-17 or 39, (ii) a functional fragment thereof, or (iii) a sequence at least 80% identical to (i) or (ii), does not result in greatly decreased expression of a polypeptide encoded by the mRNA in target cells as compared to expression of the polypeptide in target cells from an otherwise equivalent mRNA without the sequence of (i), (ii) or (iii). For example, the sequence of (i), (ii) or (iii), may not decrease expression of the polypeptide encoded by the mRNA in the target cells as compared to expression of the polypeptide in the target cells from an otherwise equivalent mRNA without the sequence of (i), (ii) or (iii). For example, in some embodiments, the sequence of (i), (ii) or (iii), may result in expression of a polypeptide encoded by the mRNA in target cells at a level that is at least at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of the expression of the polypeptide in target cells from an otherwise equivalent mRNA without the sequence of (i), (ii) or (iii).

In any embodiment herein, the target cells may be neural cells, muscle cells, cardiac cells, skin cells, immune cells, hematopoetic cells, cancer cells, pancreatic cells or kidney cells. In some cases, the target cells may be neural cells, e.g., cerebrum cells, brainstem cells, hippocampus cells or cerebellum cells. For example, in some embodiments, the neural cells are GABAergic cells, e.g., parvalbumin expressing cells. In some cases, the target cell may be a CNS cell, such as an excitatory neuron, a dopaminergic neuron, a glial cell, an ependymal cell, an oligodendrocyte, an astrocyte, a microglia, a motor neuron, a vascular cell, a GABAergic neuron, or a non-GABAergic neuron (e.g., a cell that does not express one or more of GAD2, GAD1, NKX2.1, DLX1, DLX5, SST and VIP), a non-PV neuron (e.g., a GABAergic neuron that does not express parvalbumin), or other CNS cells (e.g., CNS cell types that have never expressed any of PV, GAD2, GAD1, NKX2.1, DLX1, DLX5, SST and VIP).

In any embodiment, the method may further comprise administering a vector (e.g., an AAV or lentiviral vector) encoding the mRNA to a subject, e.g., wherein the mRNA encodes a therapeutic protein. In some embodiments, the method may comprise administering the mRNA to a subject.

### Expression Cassettes

A nucleic acid cassette may contain one or more additional regulatory elements (e.g., a promoter, a terminator, and/or an enhancer, etc.) that induces expression of transgene in a particular cell type, or a particular class of cell types. For instance, a cell type selective regulatory element can induce gene expression in a particular cell type relative to one or more other cell types. Alternatively or in addition, a cell type selective regulatory element can induce gene expression in a particular class of cells relative to one or more other classes of cells. In one embodiment, a cell type selective regulatory element of the invention enhances gene expression in a particular cell type, or a particular class of cells. In another embodiment, a cell type selective regulatory element suppresses gene expression in a particular cell type, or a particular class of cells. Cell type selective modulation of gene expression (e.g., enhancing or suppressing gene expression) does not require that gene expression is affected only in the target cell type or class of cells. Rather, cell type selective modulation of gene expression (e.g., enhancing or suppressing gene expression) requires only that gene expression increase, or decrease, in the target cell type relative to one or more other cell types, or classes of cells.

In one embodiment, the application provides an expression cassette comprising a promoter operably linked to nucleic acid sequence encoding an mRNA, wherein the mRNA comprises a liver de-targeting region comprising (i) SEQ ID NO: 1; (ii) a variant, functional fragment, multiple copies, or a combination thereof; or (iii) a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to any one of (i) or (ii). In certain embodiments, the promoter is a tissue selective or tissue specific promoter. In certain embodiments, the promoter is a CNS selective promoter and the mRNA encodes a therapeutic protein for a neural disease or disorder.

In one embodiment, the application provides an expression cassette comprising a promoter operably linked to nucleic acid sequence encoding an mRNA, wherein the mRNA comprises a liver de-targeting region comprising (i) SEQ ID NO: 2; (ii) a variant, functional fragment, multiple copies, or a combination thereof; or (iii) a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to any one of (i) or (ii). In certain embodiments, the promoter is a tissue selective or tissue specific promoter. In certain embodiments, the promoter is a CNS selective promoter and the mRNA encodes a therapeutic protein for a neural disease or disorder.

In one embodiment, the application provides an expression cassette comprising a promoter operably linked to nucleic acid sequence encoding an mRNA, wherein the mRNA comprises a liver de-targeting region comprising (i) SEQ ID NO: 3; (ii) a variant, functional fragment, multiple copies, or a combination thereof; or (iii) a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to any one of (i) or (ii). In certain embodiments, the promoter is a tissue selective or tissue specific promoter. In certain embodiments, the promoter is a CNS selective promoter and the mRNA encodes a therapeutic protein for a neural disease or disorder.

In one embodiment, the application provides an expression cassette comprising a promoter operably linked to nucleic acid sequence encoding an mRNA, wherein the mRNA comprises a liver de-targeting region comprising (i) SEQ ID NO: 4; (ii) a variant, functional fragment, multiple copies, or a combination thereof; or (iii) a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to any one of (i) or (ii). In certain embodiments, the promoter is a tissue selective or tissue specific promoter. In certain embodiments, the promoter is a CNS selective promoter and the mRNA encodes a therapeutic protein for a neural disease or disorder.

In one embodiment, the application provides an expression cassette comprising a promoter operably linked to nucleic acid sequence encoding an mRNA, wherein the mRNA comprises a liver de-targeting region comprising (i) SEQ ID NO: 5; (ii) a variant, functional fragment, multiple copies, or a combination thereof; or (iii) a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to any one of (i) or (ii). In certain embodiments, the promoter is a tissue selective or tissue specific promoter. In certain embodiments, the promoter is a CNS selective promoter and the mRNA encodes a therapeutic protein for a neural disease or disorder.

In one embodiment, the application provides an expression cassette comprising a promoter operably linked to nucleic acid sequence encoding an mRNA, wherein the mRNA comprises a liver de-targeting region comprising (i) SEQ ID NO: 6; (ii) a variant, functional fragment, multiple copies, or a combination thereof; or (iii) a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to any one of (i) or (ii). In certain embodiments, the promoter is a tissue selective or tissue specific promoter. In certain embodiments, the promoter is a CNS selective promoter and the mRNA encodes a therapeutic protein for a neural disease or disorder.

In one embodiment, the application provides an expression cassette comprising a promoter operably linked to nucleic acid sequence encoding an mRNA, wherein the mRNA comprises a liver de-targeting region comprising (i) SEQ ID NO: 7; (ii) a variant, functional fragment, multiple copies, or a combination thereof; or (iii) a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to any one of (i) or (ii). In certain embodiments, the promoter is a tissue selective or tissue specific promoter. In certain embodiments, the promoter is a CNS selective promoter and the mRNA encodes a therapeutic protein for a neural disease or disorder.

In one embodiment, the application provides an expression cassette comprising a promoter operably linked to nucleic acid sequence encoding an mRNA, wherein the mRNA comprises a liver de-targeting region comprising (i) SEQ ID NO: 8; (ii) a variant, functional fragment, multiple copies, or a combination thereof; or (iii) a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to any one of (i) or (ii). In certain embodiments, the promoter is a tissue selective or tissue specific promoter. In certain embodiments, the promoter is a CNS selective promoter and the mRNA encodes a therapeutic protein for a neural disease or disorder.

In one embodiment, the application provides an expression cassette comprising a promoter operably linked to nucleic acid sequence encoding an mRNA, wherein the mRNA comprises a liver de-targeting region comprising (i) SEQ ID NO: 9; (ii) a variant, functional fragment, multiple copies, or a combination thereof; or (iii) a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to any one of (i) or (ii). In certain embodiments, the promoter is a tissue selective or tissue specific promoter. In certain embodiments, the promoter is a CNS selective promoter and the mRNA encodes a therapeutic protein for a neural disease or disorder.

In one embodiment, the application provides an expression cassette comprising a promoter operably linked to nucleic acid sequence encoding an mRNA, wherein the mRNA comprises a liver de-targeting region comprising (i) SEQ ID NO: 10; (ii) a variant, functional fragment, multiple copies, or a combination thereof; or (iii) a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to any one of (i) or (ii). In certain embodiments, the promoter is a tissue selective or tissue specific promoter. In certain embodiments, the promoter is a CNS selective promoter and the mRNA encodes a therapeutic protein for a neural disease or disorder.

In one embodiment, the application provides an expression cassette comprising a promoter operably linked to nucleic acid sequence encoding an mRNA, wherein the mRNA comprises a liver de-targeting region comprising (i) SEQ ID NO: 11; (ii) a variant, functional fragment, multiple copies, or a combination thereof; or (iii) a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to any one of (i) or (ii). In certain embodiments, the promoter is a tissue selective or tissue specific promoter. In certain embodiments, the promoter is a CNS selective promoter and the mRNA encodes a therapeutic protein for a neural disease or disorder.

In one embodiment, the application provides an expression cassette comprising a promoter operably linked to nucleic acid sequence encoding an mRNA, wherein the mRNA comprises a liver de-targeting region comprising (i) SEQ ID NO: 12; (ii) a variant, functional fragment, multiple copies, or a combination thereof; or (iii) a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to any one of (i) or (ii). In certain embodiments, the promoter is a tissue selective or tissue specific promoter. In certain embodiments, the promoter is a CNS selective promoter and the mRNA encodes a therapeutic protein for a neural disease or disorder.

In one embodiment, the application provides an expression cassette comprising a promoter operably linked to nucleic acid sequence encoding an mRNA, wherein the mRNA comprises a liver de-targeting region comprising (i) SEQ ID NO: 13; (ii) a variant, functional fragment, multiple copies, or a combination thereof; or (iii) a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to any one of (i) or (ii). In certain embodiments, the promoter is a tissue selective or tissue specific promoter. In certain embodiments, the promoter is a CNS selective promoter and the mRNA encodes a therapeutic protein for a neural disease or disorder.

In one embodiment, the application provides an expression cassette comprising a promoter operably linked to nucleic acid sequence encoding an mRNA, wherein the mRNA comprises a liver de-targeting region comprising (i) SEQ ID NO: 14; (ii) a variant, functional fragment, multiple copies, or a combination thereof; or (iii) a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to any one of (i) or (ii). In certain embodiments, the promoter is a tissue selective or tissue specific promoter. In certain embodiments, the promoter is a CNS selective promoter and the mRNA encodes a therapeutic protein for a neural disease or disorder.

In one embodiment, the application provides an expression cassette comprising a promoter operably linked to nucleic acid sequence encoding an mRNA, wherein the mRNA comprises a liver de-targeting region comprising (i) SEQ ID NO: 15; (ii) a variant, functional fragment, multiple copies, or a combination thereof; or (iii) a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to any one of (i) or (ii). In certain embodiments, the promoter is a tissue selective or tissue specific promoter. In certain embodiments, the promoter is a CNS selective promoter and the mRNA encodes a therapeutic protein for a neural disease or disorder.

In one embodiment, the application provides an expression cassette comprising a promoter operably linked to nucleic acid sequence encoding an mRNA, wherein the mRNA comprises a liver de-targeting region comprising (i) SEQ ID NO: 16; (ii) a variant, functional fragment, multiple copies, or a combination thereof; or (iii) a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to any one of (i) or (ii). In certain embodiments, the promoter is a tissue selective or tissue specific promoter. In certain embodiments, the promoter is a CNS selective promoter and the mRNA encodes a therapeutic protein for a neural disease or disorder.

In one embodiment, the application provides an expression cassette comprising a promoter operably linked to nucleic acid sequence encoding an mRNA, wherein the mRNA comprises a liver de-targeting region comprising (i) SEQ ID NO: 17; (ii) a variant, functional fragment, multiple copies, or a combination thereof; or (iii) a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to any one of (i) or (ii). In certain embodiments, the promoter is a tissue selective or tissue specific promoter. In certain embodiments, the promoter is a CNS selective promoter and the mRNA encodes a therapeutic protein for a neural disease or disorder.

In one embodiment, the application provides an expression cassette comprising a promoter operably linked to nucleic acid sequence encoding an mRNA, wherein the mRNA comprises a liver de-targeting region comprising (i) SEQ ID NO: 39; (ii) a variant, functional fragment, multiple copies, or a combination thereof; or (iii) a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to any one of (i) or (ii). In certain embodiments, the promoter is a tissue selective or tissue specific promoter. In certain embodiments, the promoter is a CNS selective promoter and the mRNA encodes a therapeutic protein for a neural disease or disorder.

In one embodiment, the application provides an expression cassette comprising a promoter operably linked to nucleic acid sequence encoding an mRNA, wherein the mRNA comprises a liver de-targeting region comprising (i) at least two different sequences selected from SEQ ID NOs: 1-17 or 39; (ii) a variant, functional fragment, multiple copies, or a combination thereof; or (iii) a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to any one of (i) or (ii). In certain embodiments, the promoter is a tissue selective or tissue specific promoter. In certain embodiments, the promoter is a CNS selective promoter and the mRNA encodes a therapeutic protein for a neural disease or disorder.

In one embodiment, the application provides an expression cassette comprising a promoter operably linked to nucleic acid sequence encoding an mRNA, wherein the mRNA comprises a liver de-targeting region comprising (i) at least three different sequences selected from SEQ ID NOs: 1-17 or 39; (ii) a variant, functional fragment, multiple copies, or a combination thereof; or (iii) a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to any one of (i) or (ii). In certain embodiments, the promoter is a tissue selective or tissue specific promoter. In certain embodiments, the promoter is a CNS selective promoter and the mRNA encodes a therapeutic protein for a neural disease or disorder.

In one embodiment, the application provides an expression cassette comprising a promoter operably linked to nucleic acid sequence encoding an mRNA, wherein the mRNA comprises a liver de-targeting region comprising (i) at least four different sequences selected from SEQ ID NOs: 1-17 or 39; (ii) a variant, functional fragment, multiple copies, or a combination thereof; or (iii) a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to any one of (i) or (ii). In certain embodiments, the promoter is a tissue selective or tissue specific promoter. In certain embodiments, the promoter is a CNS selective promoter and the mRNA encodes a therapeutic protein for a neural disease or disorder.

In some embodiments, the nucleic acid cassette may comprise a CNS selective promoter that is operably linked to a polynucleotide encoding a therapeutic protein and one or more liver de-targeting elements/sequences as disclosed herein. CNS promoters are promoters that specifically modulate gene expression in one or more cells of the central nervous system. For example, CNS selective promoters may specifically modulate gene expression in one or more neurons or glial cells of the CNS. In one embodiment, CNS selective promoters specifically modulate gene expression in one or more neurons or astrocytes. In another embodiment, CNS selective promoters specifically modulate gene expression in one or more astrocytes. In certain embodiments, CNS selective promoters enhance expression in a CNS cell (e.g., a neuron, or a glial cell such as an astrocyte) relative to one or more other CNS cell types (e.g., excitatory neurons, dopaminergic neurons, microglia, motor neurons, vascular cells, non-GABAergic neurons, or other CNS cells).

Examples of CNS selective promoters include, but are not limited to: Ca2+/calmodulin-dependent kinase subunit a (CaMKII) promoters, synapsin I promoters, 67 kDa glutamic acid decarboxylase (GAD67) promoters, homeobox Dlx5/6 promoters, glutamate receptor 1 (GluR1) promoters, preprotachykinin 1 (Tac1) promoters, Neuron-specific enolase (NSE) promoters, dopaminergic receptor 1 (Drd1a) promoters, MAP1B promoters, Tα1 α-tubulin promoters, decarboxylase promoters, dopamine β-hydroxylase promoters, NCAM promoters, HES-5 promoters, α-internexin promoters, peripherin promoters, and GAP-43 promoters, and PaqR4 promoters. Suitable promoters are also described in, e.g., WO 2018/187363, which sequences are incorporated by reference herein. Other sequences may be used.

In some embodiments, the cassette may comprise a GABAergic neuron selective promoter that is operably linked to a polynucleotide encoding a therapeutic protein. GABAergic cells are inhibitory neurons which produce gamma-aminobutyric acid. GABAergic cells can be identified by markers such as the expression of glutamic acid decarboxylase 2 (GAD2), GAD1, NKX2.1, DLX1, DLX5, SST, PV and VIP. GABAergic neuron selective promoters are regulatory elements that specifically modulate gene expression in a GABAergic neuron. For example, GABAergic neuron-selective promoter enhance expression in a GABAergic neuron relative to one or more other CNS cell types (e.g., excitatory neurons, dopaminergic neurons, astrocytes, microglia, motor neurons, vascular cells, non-GABAergic neurons, or other CNS cells).

PV neuron selective promoters are promoters that specifically modulate gene expression in a PV neuron. For example, PV neuron selective promoters enhance expression in a PV neuron relative to one or more other CNS cell types.

In certain embodiments, a neuron selective promoter may be human derived or comprises a sequence that is human derived. In some cases, the promoter may be mouse derived or comprises a sequence that is mouse derived. In some cases, the promoter is non-naturally occurring or comprises a non-naturally occurring sequence. In some instances, the sequence of a promoter may be 100% human derived. In other instances, at least 5%, 10%, 20%, 30%, 40%, 50%, 60%, 70%, 80%, 90%, 95%, 98%, or 99% of the promoter sequence is human derived. For example, a promoter can have 50% of its sequence derived from human, and the remaining 50% be non-human derived (e.g., mouse derived or fully synthetic).

In some embodiments, the therapeutic protein encoded by the mRNA is associated with a neural disease or disorder. As noted above, neural diseases and disorders include those associated with one or more genetic mutations as well as those with unknown etiologies. Examples of neural diseases and disorders include conditions associated with epileptic seizures, neurodegenerative disorders, and/or neurodevelopmental disorders. Examples of neural diseases or disorders include, but are not limited to: Alpers-Huttenlocher Syndrome, Angelman Syndrome, CDKL5 Deficiency Disorder, Dravet Syndrome, Rett Syndrome, Parkinson's Disease and Parkinson's LIDS (side effect of Parkinson's medication), Alzheimer's disease, Creatine Transporter Deficiency, FOXG1 Syndrome, Fragile X Syndrome, Phelan-McDermid Syndrome, Childhood Absence Epilepsy, Childhood Epilepsy Centrotemporal Spikes (Benign Rolandic Epilepsy), Dravet Syndrome, Early Myoclonic Encephalopathy (EME), Epilepsy Eyelid Myoclonia Jeavons Syndrome, Epilepsy of Infancy with Migrating Focal Seizures, Epilepsy Myoclonic Absences, Epileptic Encephalopathy Continuous Spike and Wave During Sleep CSWS, Infantile Spasms (West Syndrome), Juvenile Myoclonic Epilepsy, Landau-Kleffner Syndrome, Lennox-Gastaut Syndrome (LGS), Myoclonic Epilepsy in Infancy, Ohtahara Syndrome, Panayiotopoulos Syndrome, Progressive Myoclonic Epilepsies, Reflex Epilepsies, Self-Limited Familial and Non-Familial Neonatal Infantile Seizures, Self-Limited Late Onset Occipital Epilepsy Gastaut Syndrome, Epilepsy Generalized Tonic Clonic Seizures Alone, Genetic Epilepsy with Febrile Seizures Plus, Juvenile Absence Epilepsy, Myoclonic Atonic Epilepsy Doose Syndrome, Sleep-related Hypermotor Epilepsy (SHE), febrile seizures, focal epilepsy, West Syndrome, Early Onset Epilepsy, Benign Familial Infantile Epilepsy, and Attention Deficit-Hyperactivity Disorder.

In these embodiments, the therapeutic protein may be (i): a protein encoded by a gene selected from: ALDH7A1, ARHGEF9, ARX, BRAT1, CACNA1A, CACNA1D, CACNB4, CDKL5, CHD2, CHRNA2, CHRNA4, CHRNB2, CLCN2, CLN, CLN2, DEPDC5, DNM1, FGF13, FMR1, FOLR1, FOXG1, GABRA1, GABRB3, GABRD, GABRG2, GBA1, GRIN2A, GRIN2B, GRN, HCN1, HCN4, KCNQ2, KCNQ3, KCNT1, KV3.1, KV3.2, KV3.3, LGI1, MECP2, MEF2C, Myoclonin1/EFHC1, NPRL2, PCDH19, PLCB1, PNKP, POLG1, PRRT2, PTEN, SCN1A, SCN1B, SCN2A, SCN2B, SCN8A, SHANK3, SLC13A5, SLC25A22, SLC2A1, SLC6A1, SLC6A8, SPTAN1, ST3GAL3, STRADA, STXBP1, SYNGAP1, TBC1D24, UBE3A, and WWOX, (ii) a protein having at least 80%, 81%, 82%, 83%, 84%, 85%, 86%, 87%, 88%, 89%, 90%, 91%, 92%, 93%, 94%, 95%, 96%, 97%, 98%, or 99% sequence identity to (i), (iii) a functional fragment of (i) or (ii), or (iv) a transcription factor which activates expression of a gene from (i). A transcription factor encoded by the mRNA may be an engineered transcription factor or a naturally occurring transcription factor.

In certain embodiments, the nucleic acid constructs described herein comprise another regulatory element in an addition to a promoter, such as, for example, sequences associated with transcription initiation or termination, enhancer sequences, and efficient RNA processing signals. Exemplary regulatory elements include, for example, an intron, an enhancer, UTR, stability element, WPRE sequence, a Kozak consensus sequence, posttranslational response element, or a polyadenylation (polyA) sequence, or a combination thereof. Regulatory elements can function to modulate gene expression at the transcriptional phase, post-transcriptional phase, or at the translational phase of gene expression. At the RNA level, regulation can occur at the level of translation (e.g., stability elements that stabilize mRNA for translation), RNA cleavage, RNA splicing, and/or transcriptional termination. In various embodiments, regulatory elements can recruit transcription factors to a coding region that increase gene expression selectivity in a cell type of interest, increase the rate at which RNA transcripts are produced, increase the stability of RNA produced, and/or increase the rate of protein synthesis from RNA transcripts.

In certain embodiments, the cassette may further comprise a polyA sequence. Suitable polyA sequences include, for example, an artificial polyA that is about 75 bp in length (PA75) (see e.g., WO 2018/126116), the bovine growth hormone polyA, SV40 early polyA signal, SV40 late polyA signal, rabbit beta globin polyA, HSV thymidine kinase polyA, protamine gene polyA, adenovirus 5 EIb polyA, growth hormone polyA, or a PBGD polyA. In certain embodiments, the polyA sequence is positioned downstream of the polynucleotide encoding a functional therapeutic protein in the nucleic acid constructs described herein.

### Vectors

Expression vectors may be used to deliver the nucleic acid molecule to a target cell via transfection or transduction. A vector may be an integrating or non-integrating vector, referring to the ability of the vector to integrate the expression cassette or transgene into the genome of the host cell. Examples of expression vectors include, but are not limited to, (a) non-viral vectors such as nucleic acid vectors including linear oligonucleotides and circular plasmids; artificial chromosomes such as human artificial chromosomes (HACs), yeast artificial chromosomes (YACs), and bacterial artificial chromosomes (BACs or PACs)); episomal vectors; transposons (e.g., PiggyBac); and (b) viral vectors such as retroviral vectors, lentiviral vectors, adenoviral vectors, and adeno-associated viral vectors.

Expression vectors may be linear oligonucleotides or circular plasmids and can be delivered to a cell via various transfection methods, including physical and chemical methods. Physical methods generally refer to methods of delivery employing a physical force to counteract the cell membrane barrier in facilitating intracellular delivery of genetic material. Examples of physical methods include the use of a needle, ballistic DNA, electroporation, sonoporation, photoporation, magnetofection, and hydroporation. Chemical methods generally refer to methods in which chemical carriers deliver a nucleic acid molecule to a cell and may include inorganic particles, lipid-based vectors, polymer-based vectors and peptide-based vectors.

In some embodiments, an expression vector is administered to a target cell using an inorganic particle. Inorganic particles may refer to nanoparticles, such as nanoparticles that are engineered for various sizes, shapes, and/or porosity to escape from the reticuloendothelial system or to protect an entrapped molecule from degradation. Inorganic nanoparticles can be prepared from metals (e.g., iron, gold, and silver), inorganic salts, or ceramics (e.g., phosphate or carbonate salts of calcium, magnesium, or silicon). The surface of these nanoparticles can be coated to facilitate DNA binding or targeted gene delivery. Magnetic nanoparticles (e.g., supermagnetic iron oxide), fullerenes (e.g., soluble carbon molecules), carbon nanotubes (e.g., cylindrical fullerenes), quantum dots and supramolecular systems may also be used.

In some embodiments, an expression vector is administered to a target cell using a cationic lipid (e.g., cationic liposome). Various types of lipids have been investigated for gene delivery, such as, for example, a lipid nano emulsion (e.g., which is a dispersion of one immiscible liquid in another stabilized by emulsifying agent) or a solid lipid nanoparticle.

In some embodiments, an expression vector is administered to a target cell using a peptide-based delivery vehicle. Peptide based delivery vehicles can have advantages of protecting the genetic material to be delivered, targeting specific cell receptors, disrupting endosomal membranes and delivering genetic material into a nucleus. In some embodiments, an expression vector is administered to a target cell using a polymer-based delivery vehicle. Polymer based delivery vehicles may comprise natural proteins, peptides and/or polysaccharides or synthetic polymers. In one embodiment, a polymer-based delivery vehicle comprises polyethylenimine (PEI). PEI can condense DNA into positively charged particles which bind to anionic cell surface residues and are brought into the cell via endocytosis. In other embodiments, a polymer based delivery vehicle may comprise poly-L-lysine (PLL), poly (DL-lactic acid) (PLA), poly (DL-lactide-co-glycoside) (PLGA), polyornithine, polyarginine, histones, protamines, dendrimers, chitosans, synthetic amino derivatives of dextran, and/or cationic acrylic polymers. In certain embodiments, polymer-based delivery vehicles may comprise a mixture of polymers, such as, for example PEG and PLL.

In certain embodiments, an expression vector may be a viral vector suitable for gene therapy. Preferred characteristics of viral gene therapy vectors or gene delivery vectors may include the ability to be reproducibly and stably propagated and purified to high titres; to mediate targeted delivery (e.g., to deliver the transgene specifically to the tissue or organ of interest without widespread vector dissemination elsewhere); and to mediate gene delivery and transgene expression without inducing harmful side effects.

Several types of viruses, for example the non-pathogenic parvovirus referred to as adeno-associated virus, have been engineered for the purposes of gene therapy by harnessing the viral infection pathway but avoiding the subsequent expression of viral genes that can lead to replication and toxicity. Such viral vectors can be obtained by deleting all, or some, of the coding regions from the viral genome, but leaving intact those sequences (e.g., terminal repeat sequences) that may be necessary for functions such as packaging the vector genome into the virus capsid or the integration of vector nucleic acid (e.g., DNA) into the host chromatin.

In various embodiments, suitable viral vectors include retroviruses (e.g., A-type, B-type, C-type, and D-type viruses), adenovirus, parvovirus (e.g. adeno-associated viruses or AAV), coronavirus, negative strand RNA viruses such as orthomyxovirus (e.g., influenza virus), rhabdovirus (e.g., rabies and vesicular stomatitis virus), paramyxovirus (e. g. measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Examples of retroviruses include avian leukosis-sarcoma virus, human T-lymphotrophic virus type 1 (HTLV-1), bovine leukemia virus (BLV), lentivirus, and spumavirus. Other viruses include Norwalk virus, togavirus, flavivirus, reoviruses, papovavirus, hepadnavirus, and hepatitis virus, for example. Viral vectors may be classified into two groups according to their ability to integrate into the host genome—integrating and non-integrating. Oncoretroviruses and lentiviruses can integrate into host cellular chromatin while adenoviruses, adeno-associated viruses, and herpes viruses predominantly persist in the cell nucleus as extrachromosomal episomes.

In certain embodiments, a suitable viral vector is a retroviral vector. Retroviruses refer to viruses of the family Retroviridae. Examples of retroviruses include oncoretroviruses, such as murine leukemia virus (MLV), and lentiviruses, such as human immunodeficiency virus 1 (HIV-1). Retroviral genomes are single-stranded (ss) RNAs and comprise various genes that may be provided in cis or trans. For example, retroviral genome may contain cis-acting sequences such as two long terminal repeats (LTR), with elements for gene expression, reverse transcription and integration into the host chromosomes. Other components include the packaging signal (psi or y), for the specific RNA packaging into newly formed virions and the polypurine tract (PPT), the site of the initiation of the positive strand DNA synthesis during reverse transcription. In addition, the retroviral genome may comprise gag, pol and env genes. The gag gene encodes the structural proteins, the pol gene encodes the enzymes that accompany the ssRNA and carry out reverse transcription of the viral RNA to DNA, and the env gene encodes the viral envelope. Generally, the gag, pol and env are provided in trans for viral replication and packaging.

In certain embodiments, a retroviral vector provided herein may be a lentiviral vector. At least five serogroups or serotypes of lentiviruses are recognized. Viruses of the different serotypes may differentially infect certain cell types and/or hosts. Lentiviruses, for example, include primate retroviruses and non-primate retroviruses. Primate retroviruses include HIV and simian immunodeficiency virus (SIV). Non-primate retroviruses include feline immunodeficiency virus (FIV), bovine immunodeficiency virus (BIV), caprine arthritis-encephalitis virus (CAEV), equine infectious anemia virus (EIAV) and visnavirus. Lentiviruses or lentivectors may be capable of transducing quiescent cells. As with oncoretrovirus vectors, the design of lentivectors may be based on the separation of cis- and trans-acting sequences.

In exemplary embodiments, a viral vector provided herein is an adeno-associated virus (AAV). AAV is a small, replication-defective, non-enveloped animal virus that infects humans and some other primate species. AAV is not known to cause human disease and induces a mild immune response. AAV vectors can also infect both dividing and quiescent cells without integrating into the host cell genome.

The AAV genome consists of a linear single stranded DNA which is ~4.7 kb in length. The genome consists of two open reading frames (ORF) flanked by an inverted terminal repeat (ITR) sequence that is about 145 bp in length. The ITR consists of a nucleotide sequence at the 5′ end (5′ ITR) and a nucleotide sequence located at the 3′ end (3′ ITR) that contain palindromic sequences. The ITRs function in cis by folding over to form T-shaped hairpin structures by complementary base pairing that function as primers during initiation of DNA replication for second strand synthesis. The two open reading frames encode for rep and cap genes that are involved in replication and packaging of the virion. In an exemplary embodiment, an AAV vector provided herein does not contain the rep or cap genes. Such genes may be provided in trans for producing virions as described further below.

In certain embodiments, an AAV vector may include a stuffer nucleic acid. In some embodiments, the stuffer nucleic acid may encode a green fluorescent protein or antibiotic resistance gene such as kanamycin or ampicillin. In certain embodiments, the stuffer nucleic acid may be located outside of the ITR sequences (e.g., as compared to the polynucleotide encoding a therapeutic protein, and regulatory sequences, which are located between the 5′ and 3′ ITR sequences).

Various serotypes of AAV exist, including AAV1, AAV2, AAV3, AAV4, AAV5, AAV6, AAV7, AAV8, AAV9, AAV9.47, AAV9(hul4), AAV10, AAV11, AAV 12, AAV13, AAVrh8, AAVrhlO, AAV-DJ, and AAV-DJ8. These serotypes differ in their tropism, or the types of cells they infect. AAVs may comprise the genome and capsids from multiple serotypes (e.g., pseudotypes). For example, an AAV may comprise the genome of serotype 2 (e.g., ITRs) packaged in the capsid from serotype 5 or serotype 9. Pseudotypes may improve transduction efficiency as well as alter tropism.

In some embodiments, an AAV vector or an AAV viral particle, or virion, may be used to deliver a construct comprising a cell selective regulatory element operably linked to a polynucleotide encoding functional therapeutic protein into a cell, cell type, or tissue, and may done either in vivo, ex vivo, or in vitro. In exemplary embodiments, such an AAV vector is replication-deficient. In some embodiments, an AAV virus is engineered or genetically modified so that it can replicate and generate virions only in the presence of helper factors.

In certain embodiments, a viral vector can be selected to produce a virion having high infectivity without selectivity for a particular cell type. In certain embodiments, a viral vector can be designed to produce a virion that infects many different cell types but expression of the transgene is enhanced and/or optimized in a cell type of interest (e.g. PV neurons), and expression of the transgene is reduced and/or minimized in other non-target cell types (e.g., non-PV CNS cells). The differential expression of the transgene in different cell types can be controlled, engineered, or manipulated using different regulatory elements that are selective for one or more cell types. In some cases, one or more regulatory elements operably linked to a polynucleotide encoding a therapeutic protein enhances selective expression of the polynucleotide in a target cell, cell type, or tissue, while the one or more regulatory elements suppress transgene expression in off-target cells, cell type, or tissue, or confers significantly lower, de minimis, or statistically lower gene expression in one or more off-target cells, cell types, or tissue.

In some cases, an AAV serotype that can cross the blood brain barrier or infect cells of the CNS is preferred.

In exemplary embodiments, the application provides expression vectors that have been designed for delivery by an AAV. The AAV can be any serotype, for examples, AAV1, AAV2, AAV3, AAV4, AAV5, AAV6, AAV7, AAV8, AAV9, AAV9.47, AAV9(hul4), AAV10, AAV11, AAV 12, AAV13, AAVrh8, AAVrhlO, AAV-DJ, and AAV-DJ8, or a chimeric, hybrid, or variant AAV. The AAV can also be a self-complementary AAV (scAAV), where a “self-complementary” AAV is one in which the coding region has been designed to form an intra-molecular double-stranded DNA template. Upon infection of such vectors, rather than waiting for cell mediated synthesis of the second strand, the two complementary halves of the scAAV will associate to form one double stranded DNA (dsDNA) unit that is ready for immediate replication and transcription. The design of scAAV vectors is described in a variety of publications, including McCarty et al Gene Therapy 2001 8: 1248-54.

In certain embodiments, an expression vector designed for delivery by an AAV comprises a 5′ ITR and a 3′ ITR. In certain embodiments, an expression vector designed for delivery by an AAV comprises a 5′ ITR, a promoter, a construct as described above and a 3′ ITR. In certain embodiments, an expression vector designed for delivery by an AAV comprises a 5′ ITR, an enhancer, a promoter, a construct as described above and a 3′ ITR.

### Host Cells

In another aspect, the invention relates to a host cell comprising a nucleic acid cassette as described above. Host cells may be a bacterial cell, a yeast cell, an insect cell or a mammalian cell. In an exemplary embodiment, a host cell refers to any cell line that is susceptible to infection by a virus of interest, and amenable to culture in vitro.

In certain embodiments, a host cell provided herein may be used for ex vivo gene therapy purposes. In such embodiments, the cells are transfected with a nucleic acid molecule or expression cassette as described above subsequently transplanted into the patient or subject. Transplanted cells can have an autologous, allogenic or heterologous origin. For clinical use, cell isolation will generally be carried out under Good Manufacturing Practices (GMP) conditions. Before transplantation, cell quality and absence of microbial or other contaminants is typically checked and preconditioning, such as with radiation and/or an immunosuppressive treatment, may be carried out. Furthermore, the host cells may be transplanted together with growth factors to stimulate cell proliferation and/or differentiation.

In certain embodiments, a host cell may be used for ex vivo gene therapy into the CNS. Preferably, said cells are eukaryotic cells such as mammalian cells, these include, but are not limited to, humans, non-human primates such as apes; chimpanzees; monkeys, and orangutans, domesticated animals, including dogs and cats, as well as livestock such as horses, cattle, pigs, sheep, and goats, or other mammalian species including, without limitation, mice, rats, guinea pigs, rabbits, hamsters, and the like. A person skilled in the art will choose the more appropriate cells according to the patient or subject to be transplanted.

In certain embodiments, a host cell provided herein may be a cell with self-renewal and pluripotency properties, such as stem cells or induced pluripotent stem cells. Stem cells are preferably mesenchymal stem cells. Mesenchymal stem cells (MSCs) are capable of differentiating into at least one of an osteoblast, a chondrocyte, an adipocyte, or a myocyte and may be isolated from any type of tissue. Generally, MSCs will be isolated from bone marrow, adipose tissue, umbilical cord, or peripheral blood. Methods for obtaining thereof are well known to a person skilled in the art. Induced pluripotent stem cells (also known as iPS cells or iPSCs) are a type of pluripotent stem cell that can be generated directly from adult cells. Yamanaka et al. induced iPS cells by transferring the Oct3/4, Sox2, Klf4 and c-Myc genes into mouse and human fibroblasts, and forcing the cells to express the genes (WO 2007/069666). Thomson et al. subsequently produced human iPS cells using Nanog and Lin28 in place of Klf4 and c-Myc (WO 2008/118820).

In an exemplary embodiment, a host cell provided herein is a packaging cell. Said cells can be adherent or suspension cells. The packaging cell, and helper vector or virus or DNA construct(s) provide together in trans all the missing functions which are required for the complete replication and packaging of the viral vector.

Preferably, said packaging cells are eukaryotic cells such as mammalian cells, including simian, human, dog and rodent cells. Examples of human cells are PER.C6 cells (WO01/38362), MRC-5 (ATCC CCL-171), WI-38 (ATCC CCL-75), HEK-293 cells (ATCC CRL-1573), HeLa cells (ATCC CCL2), and fetal rhesus lung cells (ATCC CL-160). Examples of non-human primate cells are Vero cells (ATCC CCL81), COS-1 cells (ATCC CRL-1650) or COS-7 cells (ATCC CRL-1651). Examples of dog cells are MDCK cells (ATCC CCL-34). Examples of rodent cells are hamster cells, such as BHK21-F, HKCC cells, or CHO cells.

As an alternative to mammalian sources, cell lines for use in the invention may be derived from avian sources such as chicken, duck, goose, quail or pheasant. Examples of avian cell lines include avian embryonic stem cells (WO01/85938 and WO03/076601), immortalized duck retina cells (WO2005/042728), and avian embryonic stem cell derived cells, including chicken cells (WO2006/108846) or duck cells, such as EB66 cell line (WO2008/129058 & WO2008/142124).

In another embodiment, said host cell are insect cells, such as SF9 cells (ATCC CRL-1711), Sf21 cells (IPLB-Sf21), MG1 cells (BTI-TN-MG1) or High Five™ cells (BTI-TN-5B1-4).

In certain embodiments, the host cells provided herein comprise a nucleic acid construct (e.g., a plasmid) carrying the recombinant AAV vector/genome containing a cassette as described above may further comprise one or more additional nucleic acid constructs, such as, for example (i) a nucleic acid construct (e.g., an AAV helper plasmid) that encodes rep and cap genes, but does not carry ITR sequences; and/or (ii) a nucleic acid construct (e.g., a plasmid) providing the adenoviral functions necessary for AAV replication. In an exemplary embodiment, a host cell provided herein comprises: i) a nucleic acid construct or an expression vector as described above; ii) a nucleic acid construct encoding AAV rep and cap genes which does not carry the ITR sequences; and iii) a nucleic acid construct comprising adenoviral helper genes (as described further below).

In certain embodiments, the rep, cap, and adenoviral helper genes can be combined on a single plasmid (Blouin V et al. J Gene Med. 2004; 6(suppl): S223-S228; Grimm D. et al. Hum. Gene Ther. 2003; 7: 839-850). Thus, in another exemplary embodiment, a host cell provided herein comprises: i) a nucleic acid molecule or an expression cassette and ii) a plasmid encoding AAV rep and cap genes which does not carry the ITR sequences and further comprising adenoviral helper genes. Alternative methods are known. For example, the rep, cap, and adenoviral helper genes do not need to be on the same plasmid can be provide on different plasmids, or the rep and cap genes can be provided on a different plasmid to the adenoviral helper genes.

In certain embodiments, a host cell suitable for large-scale production of AAV vectors is an insect cells that can be infected with a combination of recombinant baculoviruses (Urabe et al. Hum. Gene Ther. 2002; 13: 1935-1943). For example, SF9 cells may be co-infected with three baculovirus vectors respectively expressing AAV rep, AAV cap and the AAV vector to be packaged. The recombinant baculovirus vectors will provide the viral helper gene functions required for virus replication and/or packaging.

Further guidance for the construction and production of virions for gene therapy according to the invention can be found in: Viral Vectors for Gene Therapy, Methods and Protocols. Series: Methods in Molecular Biology, Vol. 737. Merten and Al-Rubeai (Eds.); 2011 Humana Press (Springer); Gene Therapy. M. Giacca. 2010 Springer-Verlag; Heilbronn R. and Weger S. Viral Vectors for Gene Transfer: Current Status of Gene Therapeutics. In: Drug Delivery, Handbook of Experimental Pharmacology 197; M. Schafer-Korting (Ed.). 2010 Springer-Verlag; pp. 143-170; Adeno-Associated Virus: Methods and Protocols. R. O. Snyder and P. Moulllier (Eds). 2011 Humana Press (Springer); Bunning H. et al. Recent developments in adeno-associated virus technology. J. Gene Med. 2008; 10:717-733; and Adenovirus: Methods and Protocols. M. Chillon and A. Bosch (Eds.); Third. Edition. 2014 Humana Press (Springer).

### Virions & Methods of Producing Virions

In certain embodiments, the application provides viral particles comprising a viral vector. The terms “viral particle”, and “virion” are used herein interchangeably and relate to an infectious and typically replication-defective virus particle comprising the viral genome (e.g., the viral expression vector) packaged within a capsid and, as the case may be e.g., for retroviruses, a lipidic envelope surrounding the capsid. A “capsid” refers to the structure in which the viral genome is packaged. A capsid consists of several oligomeric structural subunits made of proteins. For example, AAV have an icosahedral capsid formed by the interaction of three capsid proteins: VP1, VP2 and VP3. In one embodiment, a virion provided herein is a recombinant AAV virion or rAAV virion obtained by packaging an AAV vector in a protein shell.

In certain embodiments, a recombinant AAV virion provided herein may be prepared by encapsidating an AAV genome derived from a particular AAV serotype in a viral particle formed by natural Cap proteins corresponding to an AAV of the same particular serotype. In other embodiments, an AAV viral particle provided herein comprises a viral vector comprising ITR(s) of a given AAV serotype packaged into proteins from a different serotype. See e.g., Bunning H et al. J Gene Med 2008; 10: 717-733. For example, a viral vector having ITRs from a given AAV serotype may be package into: a) a viral particle constituted of capsid proteins derived from a same or different AAV serotype (e.g. AAV2 ITRs and AAV9 capsid proteins; AAV2 ITRs and AAV8 capsid proteins; etc.); b) a mosaic viral particle constituted of a mixture of capsid proteins from different AAV serotypes or mutants (e.g. AAV2 ITRs with AAV1 and AAV9 capsid proteins); c) a chimeric viral particle constituted of capsid proteins that have been truncated by domain swapping between different AAV serotypes or variants (e.g. AAV2 ITRs with AAV8 capsid proteins with AAV9 domains); or d) a targeted viral particle engineered to display selective binding domains, enabling stringent interaction with target cell specific receptors (e.g. AAV5 ITRs with AAV9 capsid proteins genetically truncated by insertion of a peptide ligand; or AAV9 capsid proteins non-genetically modified by coupling of a peptide ligand to the capsid surface).

The skilled person will appreciate that an AAV virion provided herein may comprise capsid proteins of any AAV serotype. In one embodiment, the viral particle comprises capsid proteins from an AAV serotype selected from the group consisting of an AAV1, an AAV2, an AAV5, an AAV8, and an AAV9, which are more suitable for delivery to the CNS (M. Hocquemiller et al., Hum Gene Ther 27(7): 478-496 (2016)). In a particular embodiment, the viral particle comprises a nucleic acid construct of the invention wherein the 5′ITR and 3′ITR sequences of the nucleic acid construct are of an AAV2 serotype and the capsid proteins are of an AAV9 serotype.

Numerous methods are known in the art for production of rAAV virions, including transfection, stable cell line production, and infectious hybrid virus production systems which include adenovirus-AAV hybrids, herpesvirus-AAV hybrids (Conway, J E et al., (1997) J. Virology 71(11):8780-8789) and baculovirus-AAV hybrids. rAAV production cultures for the production of rAAV virus particles all require; 1) suitable host cells, including, for example, human-derived cell lines such as HeLa, A549, or 293 cells, or insect-derived cell lines such as SF-9, in the case of baculovirus production systems; 2) suitable helper virus function, provided by wild-type or mutant adenovirus (such as temperature sensitive adenovirus), herpes virus, baculovirus, or a plasmid construct providing helper functions; 3) AAV rep and cap genes and gene products; 4) a transgene flanked by AAV ITR sequences; and 5) suitable media and media components to support rAAV production.

In various embodiments, the host cells described herein comprise the following three components: (1) a rep gene and a cap gene, (2) genes providing helper functions, and (3) a transgene flanked by ITRs. The AAV rep gene, AAV cap gene, and genes providing helper functions can be introduced into the cell by incorporating said genes into a vector such as, for example, a plasmid, and introducing said vector into the host cell. The rep, cap and helper function genes can be incorporated into the same plasmid or into different plasmids. In a preferred embodiment, the AAV rep and cap genes are incorporated into one plasmid and the genes providing helper functions are incorporated into another plasmid. The various plasmids for creation of a host cell for virion production (e.g., comprising AAV rep and cap genes, helper functions, or a transgene) can be introduced into the cell by using any suitable method well known in the art. Examples of transfection methods include, but are not limited to, co-precipitation with calcium phosphate, DEAE-dextran, polybrene, electroporation, microinjection, liposome-mediated fusion, lipofection, retrovirus infection and biolistic transfection. In certain embodiments, the plasmids providing the rep and cap genes, the helper functions and the transgene can be introduced into the cell simultaneously. In another embodiment, the plasmids providing the rep and cap genes and the helper functions can be introduced in the cell before or after the introduction of plasmid comprising the transgene. In an exemplary embodiment, the cells are transfected simultaneously with three plasmids (e.g., a triple transfection method): (1) a plasmid comprising the transgene, (2) a plasmid comprising the AAV rep and cap genes, and (3) a plasmid comprising the genes providing the helper functions. Exemplary host cells may be 293, A549 or HeLa cells.

In other embodiments, one or more of (1) the AAV rep and cap genes, (2) genes providing helper functions, and (3) the transgene (e.g., a PV selective regulatory element operably linked to a polynucleotide encoding a therapeutic protein disclosed herein), may be carried by the packaging cell, either episomally and/or integrated into the genome of the packaging cell. In one embodiment, host cells may be packaging cells in which the AAV rep and cap genes and helper functions are stably maintained in the host cell and the host cell is transiently transfected with a plasmid containing a transgene. In another embodiment, host cells are packaging cells in which the AAV rep and cap genes are stably maintained in the host cell and the host cell is transiently transfected with a plasmid containing a transgene and a plasmid containing the helper functions. In another embodiment, host cells may be packaging cells in which the helper functions are stably maintained in the host cell and the host cell is transiently transfected with a plasmid containing a transgene and a plasmid containing rep and cap genes. In another embodiment, host cells may be producer cell lines that are stably transfected with rep and cap genes, helper functions and the transgene sequence. Exemplary packaging and producer cells may be derived from 293, A549 or HeLa cells.

In another embodiment, the producer cell line is an insect cell line (typically Sf9 cells) that is infected with baculovirus expression vectors that provide Rep and Cap proteins. This system does not require adenovirus helper genes (Ayuso E, et al., Curr. Gene Ther. 2010, 10:423-436).

The term “cap protein”, as used herein, refers to a polypeptide having at least one functional activity of a native AAV Cap protein (e.g. VP1, VP2, VP3). Examples of functional activities of cap proteins include the ability to induce formation of a capsid, facilitate accumulation of single-stranded DNA, facilitate AAV DNA packaging into capsids (i.e. encapsidation), bind to cellular receptors, and facilitate entry of the virion into host cells. In principle, any Cap protein can be used in the context of the present invention.

Cap proteins have been reported to have effects on host tropism, cell, tissue, or organ specificity, receptor usage, infection efficiency, and immunogenicity of AAV viruses. Accordingly, an AAV cap for use in an rAAV may be selected taking into consideration, for example, the subject's species (e.g. human or non-human), the subject's immunological state, the subject's suitability for long or short-term treatment, or a particular therapeutic application (e.g. treatment of a particular disease or disorder, or delivery to particular cells, tissues, or organs). In certain embodiments, the cap protein is derived from the AAV of the group consisting of AAV1, AAV2, AAV5, AAV8, and AAV9 serotypes. In an exemplary embodiment, the cap protein is derived from AAV9.

In some embodiments, an AAV Cap for use in the method of the invention can be generated by mutagenesis (i.e. by insertions, deletions, or substitutions) of one of the aforementioned AAV caps or its encoding nucleic acid. In some embodiments, the AAV cap is at least 70%, 75%, 80%, 85%, 90%, 95%, 98%, or 99% or more similar to one or more of the aforementioned AAV caps.

In some embodiments, the AAV cap is chimeric, comprising domains from two, three, four, or more of the aforementioned AAV caps. In some embodiments, the AAV cap is a mosaic of VP1, VP2, and VP3 monomers originating from two or three different AAV or a recombinant AAV. In some embodiments, a rAAV composition comprises more than one of the aforementioned caps.

In some embodiments, an AAV cap for use in a rAAV virion is engineered to contain a heterologous sequence or other modification. For example, a peptide or protein sequence that confers selective targeting or immune evasion may be engineered into a cap protein. Alternatively or in addition, the cap may be chemically modified so that the surface of the rAAV is polyethylene glycolated (i.e., pegylated), which may facilitate immune evasion. The cap protein may also be mutagenized (e.g., to remove its natural receptor binding, or to mask an immunogenic epitope).

The term “rep protein”, as used herein, refers to a polypeptide having at least one functional activity of a native AAV rep protein (e.g. rep 40, 52, 68, 78). Examples of functional activities of a rep protein include any activity associated with the physiological function of the protein, including facilitating replication of DNA through recognition, binding and nicking of the AAV origin of DNA replication as well as DNA helicase activity. Additional functions include modulation of transcription from AAV (or other heterologous) promoters and site-specific integration of AAV DNA into a host chromosome. In a particular embodiment, AAV rep genes may be from the serotypes AAV1, AAV2, AAV4, AAV5, AAV6, AAV7, AAV8, AAV9, AAV10 or AAVrh10; more preferably from an AAV serotype selected from the group consisting of AAV1, AAV2, AAV5, AAV8, and AAV9.

In some embodiments, an AAV rep protein for use in the method of the invention can be generated by mutagenesis (i.e. by insertions, deletions, or substitutions) of one of the aforementioned AAV reps or its encoding nucleic acid. In some embodiments, the AAV rep is at least 70%, 75%, 80%, 85%, 90%, 95%, 98%, or 99% or more similar to one or more of the aforementioned AAV reps.

The expressions “helper functions” or “helper genes”, as used herein, refer to viral proteins upon which AAV is dependent for replication. The helper functions include those proteins required for AAV replication including, without limitation, those proteins involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (other than herpes simplex virus type-1), and vaccinia virus. Helper functions include, without limitation, adenovirus E1, E2a, VA, and E4 or herpesvirus UL5, ULB, UL52, and UL29, and herpesvirus polymerase. In a preferred embodiment, the proteins upon which AAV is dependent for replication are derived from adenovirus.

In some embodiments, a viral protein upon which AAV is dependent for replication for use in the method of the invention can be generated by mutagenesis (i.e. by insertions, deletions, or substitutions) of one of the aforementioned viral proteins or its encoding nucleic acid. In some embodiments, the viral protein is at least 70%, 75%, 80%, 85%, 90%, 95%, 98%, or 99% or more similar to one or more of the aforementioned viral proteins.

Methods for assaying the functions of cap proteins, rep proteins and viral proteins upon which AAV is dependent for replication are well known in the art.

Host cells for expressing a transgene of interest may be grown under conditions adequate for assembly of the AAV virions. In certain embodiments, host cells are grown for a suitable period of time in order to promote the assembly of the AAV virions and the release of virions into the media. Generally, cells may be grown for about 24 hours, about 36 hours, about 48 hours, about 72 hours, about 4 days, about 5 days, about 6 days, about 7 days, about 8 days, about 9 days, or up to about 10 days. After about 10 days (or sooner, depending on the culture conditions and the particular host cell used), the level of production generally decreases significantly. Generally, time of culture is measured from the point of viral production. For example, in the case of AAV, viral production generally begins upon supplying helper virus function in an appropriate host cell as described herein. Generally, cells are harvested about 48 to about 100, preferably about 48 to about 96, preferably about 72 to about 96, preferably about 68 to about 72 hours after helper virus infection (or after viral production begins).

rAAV production cultures can be grown under a variety of conditions (over a wide temperature range, for varying lengths of time, and the like) suitable to the particular host cell being utilized. rAAV production cultures include attachment-dependent cultures which can be cultured in suitable attachment-dependent vessels such as, for example, roller bottles, hollow fiber filters, microcarriers, and packed-bed or fluidized-bed bioreactors. rAAV vector production cultures may also include suspension-adapted host cells such as HeLa, 293, and SF-9 cells which can be cultured in a variety of ways including, for example, spinner flasks, stirred tank bioreactors, and disposable systems such as the Wave bag system.

Suitable media known in the art may be used for the production of rAAV virions. These media include, without limitation, media produced by Hyclone Laboratories and JRH including Modified Eagle Medium (MEM), Dulbecco's Modified Eagle Medium (DMEM), each of which is incorporated herein by reference in its entirety. In certain embodiments, rAAV production culture media may be supplemented with serum or serum-derived recombinant proteins at a level of 0.5%-20% (v/v or w/v). Alternatively, rAAV vectors may be produced in serum-free conditions which may also be referred to as media with no animal-derived products.

After culturing the host cells to allow AAV virion production, the resulting virions may be then be harvested and purified. In certain embodiments, the AAV virions can be obtained from (1) the host cells of the production culture by lysis of the host cells, and/or (2) the culture medium of said cells after a period of time post-transfection, preferably 72 hours. The rAAV virions may be harvested from the spent media from the production culture, provided the cells are cultured under conditions that cause release of rAAV virions into the media from intact cells (see e.g., U.S. Pat. No. 6,566,118). Suitable methods of lysing cells are also known in the art and include for example multiple freeze/thaw cycles, sonication, microfluidization, and treatment with chemicals, such as detergents and/or proteases.

After harvesting, the rAAV virions may be purified. The term “purified” as used herein includes a preparation of rAAV virions devoid of at least some of the other components that may also be present where the rAAV virions naturally occur or are initially prepared from. Thus, for example, purified rAAV virions may be prepared using an isolation technique to enrich it from a source mixture, such as a culture lysate or production culture supernatant. Enrichment can be measured in a variety of ways, such as, for example, by the proportion of DNase-resistant particles (DRPs) or genome copies (gc) present in a solution, or by infectivity, or it can be measured in relation to a second, potentially interfering substance present in the source mixture, such as contaminants, including production culture contaminants or in-process contaminants, including helper virus, media components, and the like.

In certain embodiments, the rAAV production culture harvest may be clarified to remove host cell debris. In some embodiments, the production culture harvest may be clarified using a variety of standard techniques, such as, centrifugation or filtration through a filter of 0.2 μm or greater pore size (e.g., a cellulose acetate filter or a series of depth filters).

In certain embodiments, the rAAV production culture harvest is further treated with Benzonase™ to digest any high molecular weight DNA present in the production culture. In some embodiments, the Benzonase™ digestion is performed under standard conditions, for example, a final concentration of 1-2.5 units/ml of Benzonase™ at a temperature ranging from ambient to 37° C. for a period of 30 minutes to several hours.

In certain embodiments, the rAAV virions may be isolated or purified using one or more of the following purification steps: equilibrium centrifugation; flow-through anionic exchange filtration; tangential flow filtration (TFF) for concentrating the rAAV particles; rAAV capture by apatite chromatography; heat inactivation of helper virus; rAAV capture by hydrophobic interaction chromatography; buffer exchange by size exclusion chromatography (SEC); nanofiltration; and rAAV capture by anionic exchange chromatography, cationic exchange chromatography, or affinity chromatography. These steps may be used alone, in various combinations, or in different orders. Methods to purify rAAV particles are found, for example, in Xiao et al., (1998) Journal of Virology 72:2224-2232; U.S. Pat. Nos. 6,989,264 and 8,137,948; and WO 2010/148143.

In certain embodiments, purified AAV virions can be dialyzed against PBS, filtered and stored at −80° C. Titers of viral genomes can be determined by quantitative PCR using linearized plasmid DNA as standard curve (see e.g., Lock M, et al., Hum. Gene Ther. 2010; 21:1273-1285).

### Pharmaceutical Compositions

In certain embodiments, the application provides compositions comprising a nucleic acid cassette, e.g., an expression cassette, e.g., an rAAV comprising an expression cassette, described above or an RNA, e.g., an mRNA, encoded by the same, and a pharmaceutically acceptable carrier. In some embodiments, a virion containing the cassette and a pharmaceutically acceptable carrier is provided. In exemplary embodiments, such compositions are suitable for gene therapy applications. Pharmaceutical compositions are preferably sterile and stable under conditions of manufacture and storage. Sterile solutions may be accomplished, for example, by filtration through sterile filtration membranes.

Acceptable carriers and excipients in the pharmaceutical compositions are preferably nontoxic to recipients at the dosages and concentrations employed. Acceptable carriers and excipients may include buffers such as phosphate, citrate, HEPES, and TAE, antioxidants such as ascorbic acid and methionine, preservatives such as hexamethonium chloride, octadecyldimethylbenzyl ammonium chloride, resorcinol, and benzalkonium chloride, proteins such as human serum albumin, gelatin, dextran, and immunoglobulins, hydrophilic polymers such as polyvinylpyrrolidone, amino acids such as glycine, glutamine, histidine, and lysine, and carbohydrates such as glucose, mannose, sucrose, and sorbitol. Pharmaceutical compositions of the disclosure can be administered parenterally in the form of an injectable formulation. Pharmaceutical compositions for injection can be formulated using a sterile solution or any pharmaceutically acceptable liquid as a vehicle. Pharmaceutically acceptable vehicles include, but are not limited to, sterile water and physiological saline.

The pharmaceutical compositions of the disclosure may be prepared in microcapsules, such as hydroxylmethylcellulose or gelatin-microcapsules and polymethylmethacrylate microcapsules. The pharmaceutical compositions of the disclosure may also be prepared in other drug delivery systems such as liposomes, albumin microspheres, microemulsions, nano-particles, and nanocapsules. The pharmaceutical composition for gene therapy can be in an acceptable diluent, or can comprise a slow release matrix in which the gene delivery vehicle is imbedded.

Pharmaceutical compositions provided herein may be formulated for parenteral administration, subcutaneous administration, intravenous administration, intramuscular administration, intra-arterial administration, intraparenchymal administration, intrathecal administration, intra-cisterna magna administration, intracerebroventricular administration, or intraperitoneal administration. The pharmaceutical composition may also be formulated for, or administered via, nasal, spray, oral, aerosol, rectal, or vaginal administration. In one embodiment, a pharmaceutical composition provided herein is administered to the CNS or cerebral spinal fluid (CSF), i.e. by intraparenchymal injection, intrathecal injection, intra-cisterna magna injection, or intracerebroventricular injection. The tissue target may be specific, for example the CNS, or it may be a combination of several tissues, for example the muscle and CNS tissues. Exemplary tissue or other targets may include liver, skeletal muscle, heart muscle, adipose deposits, kidney, lung, vascular endothelium, epithelial, hematopoietic cells, cancer cells, CNS and/or CSF. In a preferred embodiment, a pharmaceutical composition provided herein is administered to the CNS or CSF injection, i.e. by intraparenchymal injection, intrathecal injection, intra-cisterna magna injection, or intracerebroventricular injection. One or more of these methods may be used to administer a pharmaceutical composition of the disclosure.

In certain embodiments, a pharmaceutical composition provided herein comprises an “effective amount” or a “therapeutically effective amount.” As used herein, such amounts refer to an amount effective, at dosages and for periods of time necessary to achieve the desired therapeutic result.

The dosage of the pharmaceutical compositions of the disclosure depends on factors including the route of administration, the disease to be treated, and physical characteristics (e.g., age, weight, general health) of the subject. Dosage may be adjusted to provide the optimum therapeutic response. Typically, a dosage may be an amount that effectively treats the disease without inducing significant toxicity. In certain embodiments, the pharmaceutical composition may be formed in a unit dose as needed.

Pharmaceutical compositions of the disclosure may be administered to a subject in need thereof, as medically necessary. In an exemplary embodiment, a single administration is sufficient. In one embodiment, the pharmaceutical composition is suitable for use in human subjects and is administered by intraparenchymal injection, intrathecal injection, intra-cisterna magna injection, or intracerebroventricular injection. In one embodiment, the pharmaceutical composition is delivered via a peripheral vein by bolus injection. In other embodiments, the pharmaceutical composition is delivered via a peripheral vein by infusion.

In another aspect, the application further provides a kit comprising a nucleic acid molecule, vector, host cell, virion or pharmaceutical composition as described herein in one or more containers. A kit may include instructions or packaging materials that describe how to administer a nucleic acid molecule, vector, host cell or virion contained within the kit to a patient. Containers of the kit can be of any suitable material, e.g., glass, plastic, metal, etc., and of any suitable size, shape, or configuration. In certain embodiments, the kits may include one or more ampoules or syringes that contain a nucleic acid molecule, vector, host cell, virion or pharmaceutical composition in a suitable liquid or solution form.

### Methods of Treatment

The present nucleic acid cassette, expression cassette, expression vector, viral vector, viral particle or pharmaceutical composition may be used for the treatment of a variety of disorders, e.g., neurological disorders. In some embodiments, a chemical, protein, or nucleic acid molecule of the invention may be used to treat or ameliorate one or more symptoms associated with a mutation in a gene, or an under-expressed or non-expressed gene in a subject. In certain embodiments, the treatment may be treating a subject via gene therapy wherein the gene therapy is administered directly to the subject (e.g., directly to the CNS) of a subject in need thereof or systematically via injection and/or infusion. The therapy may be formulated for parenteral administration, subcutaneous administration, intravenous administration, intramuscular administration, intra-arterial administration, intraparenchymal administration, intrathecal administration, intra-cisterna magna administration, intracerebroventricular administration, or intraperitoneal administration, or via, nasal, spray, oral, aerosol, rectal, or vaginal administration, e.g., by intraparenchymal injection, intrathecal injection, intra-cisterna magna injection, or intracerebroventricular injection. The tissue target may be specific, for example the CNS, or it may be a combination of several tissues.

In any embodiment herein, the target cells may be neural cells, muscle cells, cardiac cells, skin cells, immune cells, hematopoetic cells, cancer cells, pancreatic cells, or kidney cells. In some cases, the target cells may be neural cells, e.g., cerebrum cells, brainstem cells, hippocampus cells, or cerebellum cells. For example, in some embodiments, the neural cells are GABAergic cells, e.g., parvalbumin expressing cells. In some cases, the target cell may be a CNS cell, such as an excitatory neuron, a dopaminergic neuron, a glial cell, an ependymal cell, an oligodendrocyte, an astrocyte, a microglia, a motor neuron, a vascular cell, a GABAergic neuron, or a non-GABAergic neuron (e.g., a cell that does not express one or more of GAD2, GAD1, NKX2.1, DLX1, DLX5, SST and VIP), a non-PV neuron (e.g., a GABAergic neuron that does not express parvalbumin), or other CNS cells (e.g., CNS cell types that have never expressed any of PV, GAD2, GAD1, NKX2.1, DLX1, DLX5, SST and VIP).

In any embodiment, the present therapy may be used to increase the production or expression of a target protein in a cell, such as a GABA neuron or a parvalbumin neuron.

In certain embodiments, a treatment provided herein does not result in an adverse reaction for the subject. Treatment with a nucleic acid molecule, expression vector, pharmaceutical composition, or virion described herein can cause fewer, or less severe, adverse reactions in a subject than treatment with a similar gene therapy containing the same transgene linked to a non-parvalbumin neuron-selective regulatory element.

### Sequence Table

The following table (Table 1) provides the sequence referred to in other parts of this disclosure.

SEQ ID NOS 1-17 and 39 provide exemplary liver de-targeting elements/sequences that may be in an RNA transcript encoded in a nucleic acid cassette of the present disclosure.

SEQ ID NOS 18-34 and 40 provide liver de-targeting sequences that may be in a nucleic acid cassette (a DNA cassette) encoding an RNA transcript (e.g., an mRNA) that contains any of SEQ ID NOS 1-17 or 39.

Additional sequences are provided as indicated in Table 1.

| SEQ ID | | |
|---|---|---|
| NO | Description | Sequence |
| 1 | Test sequence 1 | ACAGUUCUUCAACUGGCAGCUU |
| | (RNA) | (SEQ ID NO: 1) |
| 2 | Test sequence 2 | CCACGACCUAAUCCUAACU (SEQ ID |
| | (RNA) | NO: 2) |
| 3 | Test sequence 3 | GGGAGCUAGGUUGCCAUGUGC (SEQ |
| | (RNA) | ID NO: 3) |
| 4 | Test sequence 4 | CUACCUGCACUGUAAGCACUUUG |
| | (RNA) | (SEQ ID NO: 4) |
| 5 | Test sequence 5 | CUAGUGGUCCUAAACAUUUCAC |
| | (RNA) | (SEQ ID NO: 5) |
| 6 | Test sequence 6 | AUUUAGUGUGAUAAUGGCGU (SEQ |
| | (RNA) | ID NO: 6) |
| 7 | Test sequence 7 | CAAACACCAUUGUCACACUCCA |
| | (RNA) | (SEQ ID NO: 7) |
| 8 | Test sequence 8 | AAGCUGCCAGUUGAAGAACUG (SEQ |
| | (RNA) | ID NO: 8) |
| 9 | Test sequence 9 | GCACAUGGCAACCUAGCUCC (SEQ |
| | (RNA) | ID NO: 9) |
| 10 | Test sequence 10 | AAAGUGCUUACAGUGCAGGUAG |
| | (RNA) | (SEQ ID NO: 10) |
| 11 | Test sequence 11 | ACGCCAUUAUCACACUAAAU (SEQ |
| | (RNA) | ID NO: 11) |
| 12 | Test sequence 12 | GAGAUCAUAUGCAUAUGUGUAGGG |
| | (RNA) | CUGGAGCGUGUGUGUGUCUUGAGA |
| | | UUGUGUGUGUUGCAGUCAUCAUAU |
| | | CUAUGUGUUACAGAUUGUGUAUGU |
| | | UAGCCUUGUGUAUGUGUGCUUGAU |
| | | UGAGGUGGUGUAUUUGGGUUGAAA |
| | | UUGUGUCAUAUGUGUGUGCUAUCC |
| | | AUCUCGUGU (SEQ ID NO: 12) |
| 13 | Test sequence 13 | GCAGAAGAGGUAAGGGAGUGAGGC |
| | (RNA) | ACUCCUAUCCCAGUCUCCCAGGUU |
| | | UGGUUGAGGGCUCCCCAAGGCAGG |
| | | GCAAGAUAGCGGCCCUGUCACUGA |
| | | CCCUGGCCUGUGGUGGUCUGAGCU |
| | | GGGGAGGGAAGGACACCAAUGAAU |
| | | CAGCUUGGGACCUCUUUAGGCCUU |
| | | CCCCUUUUCCUC (SEQ ID NO: 13) |
| 14 | Test sequence 14 | GCAUUAAAACACACAUACAAACAA |
| | (RNA) | AAUCAAAAACACUGCGGACUUUCA |
| | | CUCAAGCUGGUCUUUCUUCCCCAG |
| | | UGUAAGGCAAUCCUGCCUACUAAC |
| | | AACACCAACAACAAAACACUCCAU |
| | | CUGUGAAAGCUGACGCAGUUAAGG |
| | | GGGCUAGGCAGGGCAUUUGUGCCA |
| | | ACUAAGAAUCACCAGAUACCCACC |
| | | AUAAGUACCUAUCGCAGUUUUGAA |
| | | GUCGUUUCUCCC (SEQ ID NO: 14) |
| 15 | Test sequence 15 | CAACCAUCACGUCAGGCUGCCCAU |
| | (RNA) | CCAAUAGACUCCUGGGAUGGGGCA |
| | | GCCAACCCUGGCUCAUCUCAUCUG |
| | | CCGCUUGGUGCGUGUGCGUGUGCG |
| | | UGCAUGUGCGUGUGCGUGUGUGCA |
| | | GGGGUGAGAAUCUGGCAGAUGGUG |
| | | CCUCUGCCUGCUCUUCUUCGCCUCC |
| | | UUUAUUUA (SEQ ID NO: 15) |
| 16 | Test sequence 16 | UGGCCCUGCGCAUGCUGAAAUAAC |
| | (RNA) | UGGAACCCAGCCUCUCCUCCUACAC |
| | | CGGCCUACCCAUCUGGGCCCAAGA |
| | | GCUGCACUCACACUCCUACAACGA |
| | | AGGACAAACUGUCCAGGUCGGAGG |
| | | GAUCACGAGACACAGAACCUGGAG |
| | | GGGUGUG (SEQ ID NO: 16) |
| 17 | Test sequence 17 | UCUGACUUGAGAGCUCCCCCAGUC |
| | (RNA) | AGAUCUCAGGCUUGUCCCCCUGUC |
| | | AGCUGCCUCCAGAAGGGAAGGUAG |
| | | CCAGUGCCUGAGAAGACAGUCCCU |
| | | UUUCUACCCACCGCACUCCAUAACC |
| | | UCCAUCUUCUCCCACACUGAUGGC |
| | | GAGCAGCCCCUGAGCACUUUCUGG |
| | | GACUGGGAGACUGCUUGGUGUUCC |
| | | CUGAGGACAAGAGACAUCCUGACA |
| | | GUGUUGGGCA (SEQ ID NO: 17) |
| 18 | Test sequence 1 | ACAGTTCTTCAACTGGCAGCTT (SEQ |
| | (DNA) | ID NO: 18) |
| 19 | Test sequence 2 | CCACGACCTAATCCTAACT (SEQ ID |
| | (DNA) | NO: 19) |
| 20 | Test sequence 3 | GGGAGCTAGGTTGCCATGTGC (SEQ |
| | (DNA) | ID NO: 20) |
| 21 | Test sequence 4 | CTACCTGCACTGTAAGCACTTTG |
| | (DNA) | (SEQ ID NO: 21) |
| 22 | Test sequence 5 | CTAGTGGTCCTAAACATTTCAC (SEQ |
| | (DNA) | ID NO: 22) |
| 23 | Test sequence 6 | ATTTAGTGTGATAATGGCGT (SEQ ID |
| | (DNA) | NO: 23) |
| 24 | Test sequence 7 | CAAACACCATTGTCACACTCCA (SEQ |
| | (DNA) | ID NO: 24) |
| 25 | Test sequence 8 | AAGCTGCCAGTTGAAGAACTG (SEQ |
| | (DNA) | ID NO: 25) |
| 26 | Test sequence 9 | GCACATGGCAACCTAGCTCC (SEQ |
| | (DNA) | ID NO: 26) |
| 27 | Test sequence 10 | AAAGTGCTTACAGTGCAGGTAG |
| | (DNA) | (SEQ ID NO: 27) |
| 28 | Test sequence 11 | ACGCCATTATCACACTAAAT (SEQ ID |
| | (DNA) | NO: 28) |
| 29 | Test sequence 12 | GAGATCATATGCATATGTGTAGGGC |
| | (DNA) | TGGAGCGTGTGTGTGTCTTGAGATT |
| | | GTGTGTGTTGCAGTCATCATATCTAT |
| | | GTGTTACAGATTGTGTATGTTAGCCT |
| | | TGTGTATGTGTGCTTGATTGAGGTG |
| | | GTGTATTTGGGTTGAAATTGTGTCAT |
| | | ATGTGTGTGCTATCCATCTCGTGT |
| | | (SEQ ID NO: 29) |
| 30 | Test sequence 13 | GCAGAAGAGGTAAGGGAGTGAGGC |
| | (DNA) | ACTCCTATCCCAGTCTCCCAGGTTTG |
| | | GTTGAGGGCTCCCCAAGGCAGGGCA |
| | | AGATAGCGGCCCTGTCACTGACCCT |
| | | GGCCTGTGGTGGTCTGAGCTGGGGA |
| | | GGGAAGGACACCAATGAATCAGCTT |
| | | GGGACCTCTTTAGGCCTTCCCCTTTT |
| | | CCTC (SEQ ID NO: 30) |
| 31 | Test sequence 14 | GCATTAAAACACACATACAAACAAA |
| | (DNA) | ATCAAAAACACTGCGGACTTTCACT |
| | | CAAGCTGGTCTTTCTTCCCCAGTGTA |
| | | AGGCAATCCTGCCTACTAACAACAC |
| | | CAACAACAAAACACTCCATCTGTGA |
| | | AAGCTGACGCAGTTAAGGGGGCTAG |
| | | GCAGGGCATTTGTGCCAACTAAGAA |
| | | TCACCAGATACCCACCATAAGTACC |
| | | TATCGCAGTTTTGAAGTCGTTTCTCC |
| | | C (SEQ ID NO: 31) |
| 32 | Test sequence 15 | CAACCATCACGTCAGGCTGCCCATC |
| | (DNA) | CAATAGACTCCTGGGATGGGGCAGC |
| | | CAACCCTGGCTCATCTCATCTGCCG |
| | | CTTGGTGCGTGTGCGTGTGCGTGCA |
| | | TGTGCGTGTGCGTGTGTGCAGGGGT |
| | | GAGAATCTGGCAGATGGTGCCTCTG |
| | | CCTGCTCTTCTTCGCCTCCTTTATTT |
| | | A (SEQ ID NO: 32) |
| 33 | Test sequence 16 | TGGCCCTGCGCATGCTGAAATAACT |
| | (DNA) | GGAACCCAGCCTCTCCTCCTACACC |
| | | GGCCTACCCATCTGGGCCCAAGAGC |
| | | TGCACTCACACTCCTACAACGAAGG |
| | | ACAAACTGTCCAGGTCGGAGGGATC |
| | | ACGAGACACAGAACCTGGAGGGGT |
| | | GTG (SEQ ID NO: 33) |
| 34 | Test sequence 17 | TCTGACTTGAGAGCTCCCCCAGTCA |
| | (DNA) | GATCTCAGGCTTGTCCCCCTGTCAG |
| | | CTGCCTCCAGAAGGGAAGGTAGCCA |
| | | GTGCCTGAGAAGACAGTCCCTTTTC |
| | | TACCCACCGCACTCCATAACCTCCA |
| | | TCTTCTCCCACACTGATGGCGAGCA |
| | | GCCCCTGAGCACTTTCTGGGACTGG |
| | | GAGACTGCTTGGTGTTCCCTGAGGA |
| | | CAAGAGACATCCTGACAGTGTTGGG |
| | | CA (SEQ ID NO: 34) |
| 35 | Test sequence 18 | UAAAGCUUGCCACUGAAGAACU (SEQ ID |
| | (RNA) | NO: 35) |
| 36 | Test sequence 19 | ACUGCACAAGAGCACCCAGCC (SEQ |
| | (RNA) | ID NO: 36) |
| 37 | Test sequence 20 | CUACAAGUGCCUUCACUGCAGU |
| | (RNA) | (SEQ ID NO: 37) |
| 38 | Test sequence 21 | AACUGUUGAACUGUUAAGAACCAC |
| | (RNA) | U (SEQ ID NO: 38) |
| 39 | Test sequence 22 | CUACCUGCACGAACAGCACUUUG |
| | (RNA) | (SEQ ID NO: 39) |
| 40 | Test sequence 22 | CTACCTGCACGAACAGCACTTTG |
| | (DNA) | (SEQ ID NO: 40) |
| 41 | | GCACCTGTCTAAAAGGCACTATTCC |
| | | CCATACAATGGTCCAAAGTCTGGCA |
| | | GTTACCATCCGACCGCGTTTTCTGCG |
| | | GAGCCCAGGATACTTTCTTACTTAA |
| | | GCTTGTGACGGCGGTAGAGCATGAA |
| | | A (SEQ ID NO: 41) |

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or see, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1: Detargeting Element Identification

Element selection for library generation: Elements were initially harvested from annotated 3′ untranslated regions (3′ UTR) of genomic sequences curated in the AURA database (Atlas of UTR regulatory activity; Dassi E, Re A, Leo S, Tebaldi T, Pasini L, Peroni D and Quattrone A. (2014) AURA 2: Empowering discovery of post-transcriptional networks. Translation, 2(1): e27738.), and based on proximity to liver-depleted genes according to expression data from the Genotype-Tissue Expression portal. The genes were screened for high expression in at least one non liver tissue. 100 genes were selected based on depleted expression in the liver but high expression in at least one other tissue. The genomic sequence of the 3′ UTR associated with each of these 100 genes was segmented into overlapping 127 base pair (bp) candidate elements termed “tiles”, using a sliding window distance of 25 bp.

Further elements were harvested from the Functional Annotation of the Mammalian Genome database (Fantom5, Lizio M, et al. Gateways to the FANTOM5 promoter level mammalian expression atlas. Genome Biol 16: 22 (2015). 10.1186/s13059-014-0560-6), the mircoRNA Expression and Sequence Analysis database (mESA, Koray D. Kaya, Gökhan Karakulah, Cengiz M. Yakicier, Aybar C. Acar, Ozlen Konu, mESAdb: microRNA Expression and Sequence Analysis Database, Nucleic Acids Research, Volume 39, Issue suppl_1, 1 Jan. 2011, Pages D170-D180,) and Minatel et al (Minatel B C, Martinez V D, Ng K W, et al. Large-scale discovery of previously undetected microRNAs specific to human liver. Hum Genomics. 2018; 12(1):16.). Each miRNA selected was based on a) maintaining a minimal liver expression, b) having a maximal expression in at least one other tissue type, and c) having a maximal expression in all other tissues. The selection of a miRNA was based on whether its expression in the liver was significantly different from all other tissues based on Grubb's Statistic. Expression was manually checked to ensure that the differential expression of the miRNA was higher relative to other tissues. All miRNA for consideration from the above databases were compiled and checked for redundancy between names of the miRNA.

Control element pool: The control element pool was composed of either published miRNA response elements or previously identified elements. These controls served as benchmarks and diagnostic reference points due to their predictable expression characteristics. The control elements roughly fall into three categories that include a) miRNA or 3′UTR with known expression profiles, b) various promoters with known expression profiles, and c) random sequences in the promoter position or in the 3′UTR. The miRNA, 3′UTR, and random sequence controls were driven by the same promoter used in the screen. The promoter controls contained no element or sequence in their 3′UTR region besides those needed for amplicon generation and molecular barcode identifier. Each element in the control pool was assigned a barcode, located in the 3′ UTR of the gene of interest.

Library composition: miRNA were represented as a tetramer repeat with an 8 bp spacer in between the given miRNA sequence and comprised 2000 unique elements. Those miRNA that had supporting evidence in the literature or were obtained from the FANTOM5 database were constructed into elements that contained 1, 2, 3, or 4 copies of the given single element and comprised ~350 unique elements.

Unique barcodes assigned to library elements for oligo pool synthesis: To prepare the designed library of elements for oligo pool synthesis, each candidate element's sequence was checked for the presence of restriction enzyme sites necessary for downstream cloning, and those containing these sequences were discarded. Those elements whose sequence contained the recognition sites for BsrGI, EcoRI, XbaI, AscI, and KpnI were removed from the library pool. Each of the remaining elements were assigned a barcode element. Each detargeting element and its barcode was concatenated with flanking sequences for cloning into the vector backbone. This library was synthesized as single-stranded DNA by Twist Biosciences.

Plasmid library synthesis: The experimental plasmid library was constructed from single stranded oligo pools and ligated into a common plasmid backbone. The single stranded oligos were amplified via PCR using a common set of primers and further ligated into the 3′UTR of a transgene in the screening plasmid. The plasmid is composed of the following: (minCMV promoter)-(nano Luciferase)-(MCS)-(Liver detargeting element)-(Amplicon barcode and primer sites)-(hGH polyA). The library was transformed into electrocompetent E. coli cells and was both plated onto an agar plate for Sanger Sequencing as well into a 200 ml LB liquid culture. The agar plate colonies were Sanger sequenced to validate that the elements were ligated correctly and there were no indels. The library was then isolated from the 200 mL culture using the Zymogen Maxiprep kit (Zymogen). This product was then pooled with the control spike-in plasmid library (See Control element pool section) to create the final library used for vector production.

AAV Vector preparation: All vectors were produced in adherent HEK293T cells in DMEM+10% FBS. Cells were transfected using PEI-MAX with the library of elements and helper plasmids, which include the cis ITR-containing plasmid, the trans plasmid pAAVX encoding AAV2 replication, and AAVX capsid genes and pALD-X80 the adenoviral helper plasmid. AAV was harvested from the cells and purified from the lysate using an ultra-centrifugation gradient of Iodixanol and further polished with an anion exchange column, followed by concentration and formulation in PBS with 0.001% pluronic.

Animals: The screen was carried out in adult female C57BL/6J mice (The Jackson Laboratory). Mice were obtained as 6- to 8-week-old adults and housed onsite for 3 days to acclimate to the new environment. Animals were given a standard chow diet ad libitum and on a 12 light/12dark light cycle. After injection, the mice were housed individually in a vivarium until they were sacrificed.

AAV injection: Mice (n=5) were injected by intravenous (IV) tail-vein injection and stereotaxic direct hippocampal injections. Animals were treated with Rimadyl the day prior to surgery. The mice received 3E12 gc/animal via IV in a single dose and a total of 1.8E11 gc/animal spread across four 1.5 uL injections into the left, right, dorsal, and ventral regions of the hippocampus. Animals were incubated with the virus in the vivarium for 3 weeks post injection.

Sample collection: Tissues, including hippocampus and liver, were collected directly into RNAlater (Sigma Aldrich). Samples were kept in RNAlater at 4*C for 24 hr and then transferred to −80*C till further processing.

RNA and cDNA generation: RNA from the samples was isolated with the RNeasy Mini column method using the standard protocol (Qiagen), which isolated total RNA. Total RNA concentrations were normalized prior to input into the cDNA reaction. cDNA was generated by reverse transcription using the SuperScript IV VILO kit using oligo dT primers (Thermo Fisher Scientific).

Amplicon generation: Amplicons originating from the reporter gene were amplified via PCR using a set of universal primers for all elements in the library. For each sample there were four technical replicates starting at the first amplicon PCR step. The cycle number for the amplicon PCR was optimized first with qPCR. Step1 PCR amplifies a region of the 3′UTR of the reporter mRNA under the optimized conditions. Briefly, AAV is digested with DNaseI (New England Biolabs) to remove the capsid and then used directly for Step1 PCR as above. All technical replicate samples for each biological replicate are pooled together in equal molar amounts as the final sequencing library pool.

Amplicon sequencing: Each sample was combined in equal molar amounts into the final sequencing pool, which included each biological replicates' PCR samples, the amplicons from the plasmid pool used to make the AAV, and the amplicons from the dosed AAV library. The samples were then pooled at a 60:40 molar ratio with PhiX for diversification of the sample pool. The diversified library pool was further diluted and prepared according to the specification of the Nextseq 500 High Kit (Illumina).

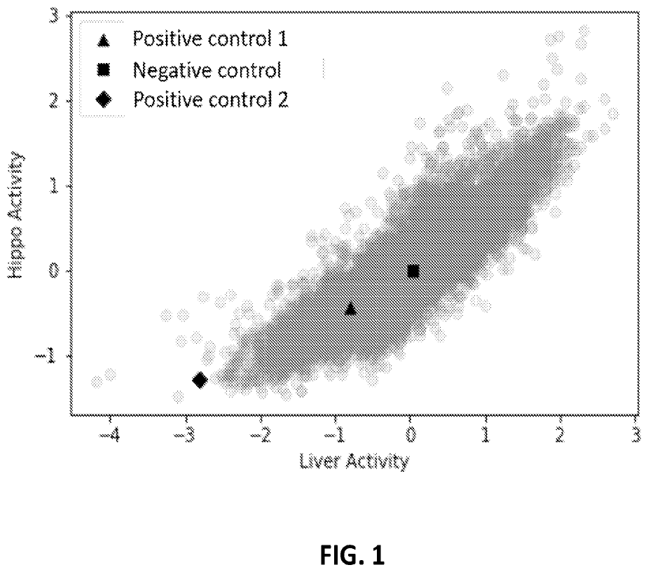

Candidate selection: A graph showing brain activity vs liver activity for the tested constructs is provided in FIG. 1. As seen in FIG. 1 many different constructs achieved decreased liver expression while maintaining brain expression, or decreased liver expression to a greater degree than brain expression. The included negative control did not affect either brain or liver expression, while the two positive controls included decreased liver expression to a greater extent than brain expression. Construct activity was assessed based on the Log 2 fold change (log 2FC) of the construct's expression activity in liver compared to its abundance in the baseline AAV pool, 2) the log 2FC of the construct's expression activity in hippocampus compared to its abundance in the baseline AAV pool, and 3) the difference between these two log 2FC values. Tissue log 2FC metrics were calculated using DESe2 (see below for more details). The log 2 fold changes in brain and liver expression of selected constructs are also provided in Table 2. The data in Table 2 shows greater reductions in liver expression than in brain expression, indicating that these elements can detarget liver expression. As shown in Table 2, elements provided herein detarget liver expression both when used singly, and when provided as tandem repeats of 2×, 3× or 4×.

TABLE 2

| | Log2 fold change | Log2 fold change |
|---|---|---|
| Description | in liver | in brain |
| Test sequence 1, 1 copy | −1.091138 | −0.502499 |
| Test sequence 1, 2 copies | −2.034107 | −1.074236 |
| Test sequence 1, 3 copies | −2.008143 | −1.313751 |
| Test sequence 1, 4 copies | −1.58071 | −1.004799 |
| Test sequence 3, 1 copy | −0.149644 | −0.115088 |
| Test sequence 3, 2 copies | −0.620037 | −0.477955 |
| Test sequence 3, 3 copies | −0.649259 | −0.352649 |
| Test sequence 3, 4 copies | −1.204078 | −0.907602 |
| Test sequence 4, 4 copies | −1.886355 | −0.388039 |
| Test sequence 5, 4 copies | −1.602891 | −0.404599 |
| Test sequence 6, 1 copy | −1.191418 | −0.151467 |
| Test sequence 6, 2 copies | −1.55778 | −0.738756 |
| Test sequence 6, 3 copies | −0.296954 | −0.119777 |
| Test sequence 7, 1 copy | −2.151987 | −0.492311 |
| Test sequence 7, 2 copies | −1.815708 | −0.787522 |
| Test sequence 7, 3 copies | −0.766551 | −0.469931 |
| Test sequence 7, 4 copies | −0.986991 | −0.333258 |
| Positive control 1 | −2.813155 | −1.276399 |
| Positive control 2 | −0.791072 | −0.443886 |
| Negative control | 0.029446 | −0.000071 |

Another detargeting element, Test sequence 22 was identified in a similarly conducted screen. As shown in Table 3, Test sequence 22 also detargeted liver expression both when used singly, and when provided as tandem repeats of 2×, 3× or 4×.

TABLE 3

| | Log2 fold change | Log2 fold change |
|---|---|---|
| Description | in liver | in brain |
| Test sequence 22, 1 copy | −1.8481975 | −0.4314376 |
| Test sequence 22, 2 copies | −2.1350587 | −0.3656623 |
| Test sequence 22, 4 copies | −2.0649475 | 0.03460468 |

Window scoring for endogenous 3′ UTR tiles: ~7,000 constructs in the de-targeting screen were sequence elements derived from the 3′ UTR regions of endogenous genes that displayed favorable expression patterns (see “Selection of endogenous 3′ UTR-derived elements” section for more details). Each endogenous 3′ UTR region is spanned by overlapping 127 bp sequence “tiles” that are each staggered by 25 bp from the previous tile. Since neighboring 3′ UTR tiles share ~80% of their sequence such tiles are expected to have similar activity profiles in general; large variations in activity between adjacent tiles may be an indicator of noisy measurement.

Aggregate “window” activity scores were calculated for each group of 3 adjacent tiles by taking the weighted average of tiles' tissue log 2FC scores, where tiles are weighted by the inverse of the tiles' log 2FC variance. Consequently, tiles that are more well-measured (i.e. less variance in the activity of the 5 constituent barcodes for the construct) were weighted more heavily towards the window average. Window activity scores were used to filter for sequence regions that consistently exhibited a favorable activity profile.

Selecting elements for validation: Several sequences were selected based on lower liver expression and preserved expression in hippocampus to further validate via IHC and ELISA.

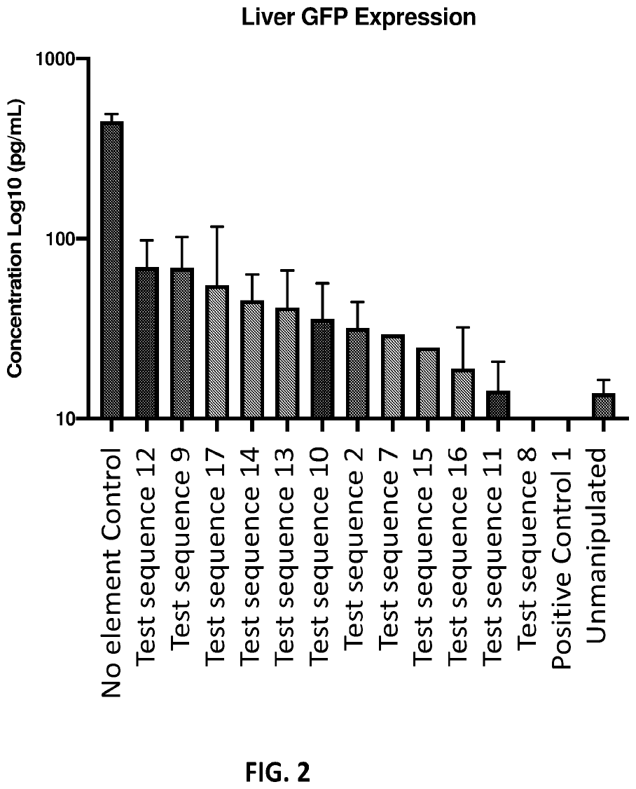

ELISA: Samples for protein analysis were from adult female C57BL/6J mice. Each mouse was dosed with 5.0E11 vg/mouse of AAV9 via intravenous injection (tail vein) for a 3 week incubation period. The vector was created as stated in AAV Vector Preparation. The GOI of the vector was as followed: (AAV2 ITR)-(EF1a(short))-mCherry-KASH-spA-(CTCF insulator)-(CMV promoter)-EGFP-KASH-(Liver Detargeting Element)-sPA-(AAV2 ITR). The Liver Detargeting Elements were selected as described above and are listed as a test sequences in Table 1 above. Both the left lateral lobe of the liver and one hemisphere of the cortex, excluding the olfactory bulb, cerebellum, and other hindbrain tissues were collected from PBS perfused mice. Samples were homogenized in Buffer PRT (Abcam, ab171581 and ab221829) with 2.8 mm ceramic beads (OMNI Intl.) in a mechanical homogenizer for 1 min at 4*C and clarified by centrifugation. Total protein was quantified using Micro BCA (ThermoFisher) and normalized in Buffer PRT. EGFP and mCherry proteins were quantified separately using ELISA (Abeam) and quantified based on a standard curve generated from a recombinant protein for each respective assay. As seen in FIG. 2, the majority of the de-targeting elements tested showed a large decrease in liver expression compared to the control.

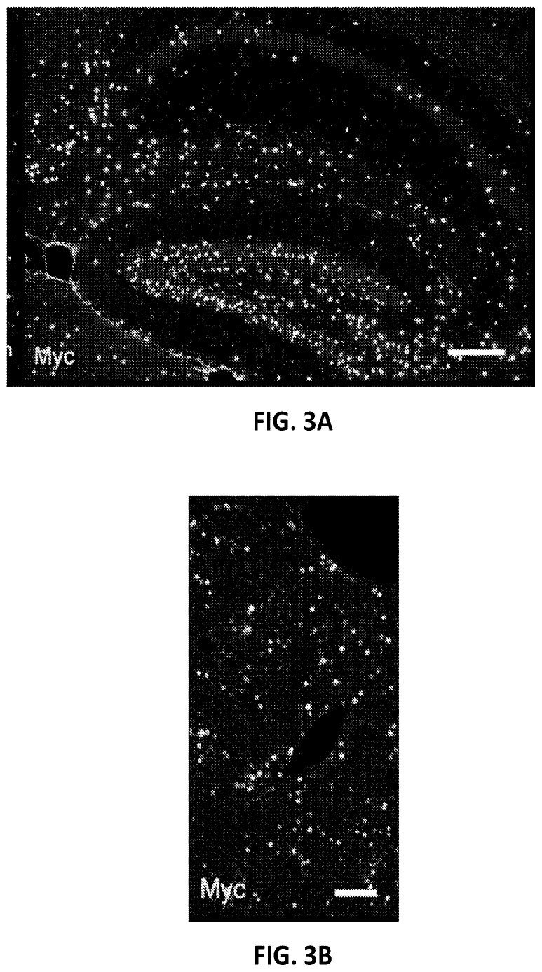

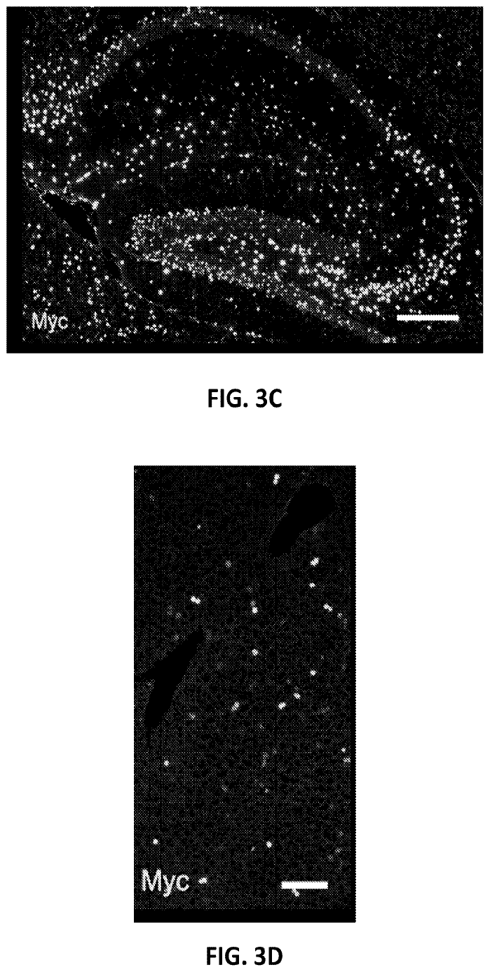

Tissue preparation and Immunohistochemical (IHC) staining: Following saline perfusion whole brain and liver tissue was collected and fixed in 4% neutral buffered formalin for 24 hrs then switched to 70% ETOH and kept at 4 C until processing. Tissue was processed for formalin fixation and paraffin embedding by an external provider. Following parasagittal embedding of the brain, 5 um sections were cut onto glass slides. Two transverse sections of the liver lobe were collected on one slide for each animal. For IHC slides were de-waxed, rehydrated and then heat induced epitope retrieval was performed for 20 min at 95 C in Citrate pH6 buffer. Slides were mounted in Shandon slide holders, washed with Phosphate Buffered Saline with Tween-20 (PBST) then incubated for 15 in antibody dilution buffer (Phosphate Buffered Saline, 1.0% Bovine Serum Albumin, 0.3% Triton-X-100) before incubating overnight at 4 C with a 1:25,000 dilution of rabbit anti-Myc [Abcam ab9106] in antibody dilution buffer. This was followed by goat anti-rabbit-HRP [Thermo A16110] for 1 hour then Opal 520 [Akoya Biosciences FP1487001KT] for 10 min. Washes were performed between each step with PBST. Nuclei were stained with 4′,6′-diamidino-2-phenylindole, dihydrochloride (DAPI) for 15 min then cover-slipped with #1.5 cover glass. FIGS. 3A and B show representative images of brain and liver expression from a mouse treated with a control vector without a detargeting element, and FIGS. 3C and 3D show images of brain and liver expression from a mouse treated with a vector encoding an RNA containing test sequence 12 (SEQ ID NO. 12).

### Example 2: In Vitro Validation of Selected Liver Detargeting Elements

Induced pluripotent stem cells (IPSCs) were used to validate the identified liver detargeting elements in human cells. Adeno-associated virus (AAV) particles were prepared using the AAVDJ serotype. Each viral preparation comprised a genome which included an EF1a promoter, a coding sequence for a enhanced green fluorescent protein fused to a KASH domain (eGFP-KASH) and either an random sequence or a liver detargeting element.

IPSC derived glutamatergic neurons and hepatocytes were plated in 24 well plates. After 48 hrs the IPSC derived cells were transduced with the different AAV constructs at a multiplicity of infection (MOI) of 5×10\hat{ }5. The cells were then incubated for an additional 72 hrs before being harvested for RNA and DNA extraction. Quantitative polymerase chain reaction was used to assay levels of eGFP-KASH mRNA, compared to an internal control (GAPDH). The results of the in vitro validation are shown in Table 4.

TABLE 4

| | Hepatocyte | Glutamatergic neuron |
|---|---|---|
| Element | expression | expression |
| Random-Seq | 1 | 1 |
| 2× Test sequence 14 | 1.41 | 1.15 |
| 4× Test sequence 4 | 0.26 | 0.52 |
| 4× Test sequence 6 | 0.19 | 1.06 |

### Example 3: Liver Detargeting in Non Human Primates

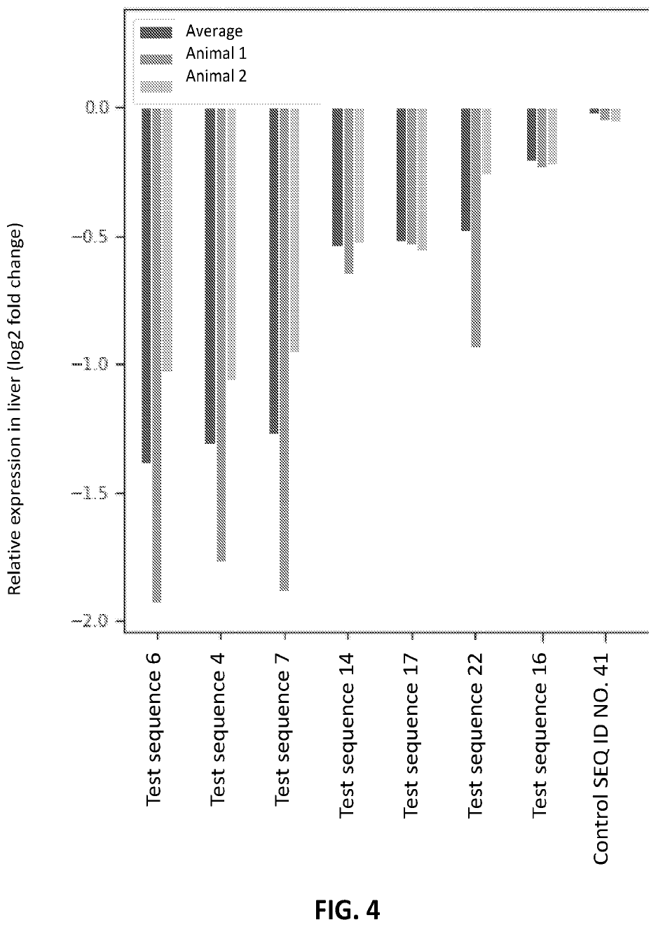

To assess conservation between mouse and non-human primate (NHP) several detargeting elements were selected for an NHP study. AAVs with different detargeting elements were prepared as described above and pooled for administration by unilateral intracerebroventricular injection at a dose of 10\hat{ }14 viral genomes/animal into juvenile cynomolgus macaques between 16-21 months of age (n=2). Animals were necropsied at approximately 50 days after treatment, and liver and brain tissue was harvested for DNA and RNA extraction. AAV-driven transcript expression was evaluated by reverse transcription droplet digital PCR analysis using vector-specific primer/probes. Total RNA expression was normalized to AAV genome copy number in each sample. FIG. 4 shows relative expression in liver (log 2 of fold change) for several different liver detargeting elements in NHPs in each of the treated animals, and averaged. As shown in FIG. 4 all the tested elements showed decreased expression in the liver compared to the control sequence, with Test sequences 4, 6 and 7 showing reductions in liver expression of more than 2 fold.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A recombinant oncolytic virus comprising one or more copies of one or more tumor-suppressive micro-RNA (miR) target sequences inserted into a locus of one or more viral genes required for viral replication.

2. The oncolytic virus of claim 1, wherein the virus is a herpes simplex virus, an adenovirus, a polio virus, a vaccinia virus, a measles virus, a vesicular stomatitis virus, an orthomyxovirus, a parvovirus, a maraba virus or a coxsackievirus.

3. The oncolytic virus of claim 1 or 2, wherein the virus is a herpes simplex virus and wherein the one or more viral genes required for viral replication is selected from the group consisting of UL1, UL5, UL6, UL7, UL8, UL9, UL11, UL12, UL14, UL15, UL17, UL18, UL19, UL20, UL22, UL25, UL26, UL26.5, UL27, UL28, UL29, UL30, UL31, UL32, UL33, UL34, UL35, UL36, UL37, UL38, UL39, UL40, UL42, UL48, UL49, UL52, UL53, UL54, ICPO, ICP4, ICP22, ICP27, ICP47, gamma-34.5, US3, US4, US5, US6, US7, US8, US9, US10, US11, and US12.

4. The oncolytic virus of any of claims 1-3, wherein the tumor-suppressive miR target sequence is a target sequence for a miR selected from Table 3.

5. The oncolytic virus of any of claims 1-4, wherein the one or more tumor-suppressive miR target sequences is incorporated into the 5′ untranslated region (UTR) or 3′ UTR of the one or more viral genes required for viral replication.

6. The oncolytic virus of any of claims 1-5, wherein replication of the virus is reduced or attenuated in a first cell compared to replication of the virus in a second cell.

7. The oncolytic virus of claim 6, wherein the first cell has an increased expression of a tumor-suppressive miR capable of binding to the one or more tumor-suppressive miR target sequences compared to the expression of the tumor-suppressive miR in the second cell.

8. The oncolytic virus of claim 7, wherein the expression level of the tumor-suppressive miR in the first cell is at least 5% greater than the expression level of the tumor-suppressive miR in the second cell.

9. The oncolytic virus of claim 6, wherein the first cell is a non-cancerous cell.

10. The oncolytic virus of claim 6, wherein the second cell has a reduced expression of a tumor-suppressive miR capable of binding to the one or more tumor-suppressive miR target sequences compared to the expression of the tumor-suppressive miR in the first cell.

11. The oncolytic virus of claim 10, wherein the expression level of the tumor-suppressive miR in the second cell is at least 5% less than the expression level of the tumor-suppressive miR in the first cell.

12. The oncolytic virus of claim 6, wherein the second cell is a cancerous cell.

13. The oncolytic virus of any of claims 1-12, comprising tumor-suppressive miR target sequences for miR-124, miR-451a, miR-143-3p, and miR-559.

14. The oncolytic virus of claim 13, for treating pancreatic, lung, and/or colon cancer.

15. The oncolytic virus of any of claims 1-12, comprising tumor-suppressive miR target sequences for miR-124, miR-451, miR-143-3p, miR-1, and miR-559.

16. The oncolytic virus of any of claims 1-12, comprising tumor-suppressive miR target sequences for miR-124, miR-451, miR-145-5p, and miR-559.

17. The oncolytic virus of claim 15 and/or 16, for treating a tumor derived from any type of cancer.

18. The oncolytic virus of any of claims 1-12, comprising tumor-suppressive miR target sequences for miR-205p, miR-141-5p, miR-31-5p, and miR-124.

19. The oncolytic virus of any of claims 1-18, wherein the tumor-suppressive miR target sequences are inserted into the ICP4, ICP27, UL19, and/or UL30 locus.

20. The oncolytic virus of any of claims 1-12, wherein the tumor-suppressive miR target sequence is a target sequence for miR-136-3p, miR-432-5p, miR-1-3p, miR-127-3p, miR-379-5p, miR-493-5p, miR-223-5p, miR-223-5p, miR-136-5p, miR-451a, miR-487b-3p, miR-370-3p, miR-410-3p, miR-431-3p, miR-4485-3p, miR-4485-5p, miR-127-5p, miR-409-3p, miR-338-3p, miR-559, miR-411-5p, miR-133a-5p, miR-143-3p, miR-376b-3p, miR-758-3p, miR-1, miR-101, miR-1180, miR-1236, miR-124-3p, miR-125b, miR-126, miR-1280, miR-133a, miR-133b, miR-141, miR-143, miR-144, miR-145, miR-155, miR-16, miR-18a, miR-192, miR-195, miR-200a, miR-200b, miR-200c, miR-203, miR-205, miR-214, miR-218, miR-23b, miR-26a, miR-29c, miR-320c, miR-34a, miR-370, miR-409-3p, miR-429, miR-451b, miR-490-5p, miR-493, miR-576-3p, and/or miR-99a.

21. The oncolytic virus of claim 20 for treating bladder cancer.

22. The oncolytic virus of any of claims 1-12, wherein the tumor-suppressive miR target sequence is a target sequence for miR-1251-5p, miR-219a-5p, miR-219a-2-3p, miR-124-3p, miR-448, miR-138-2-3p, miR-490-5p, miR-129-1-3p, miR-1264, miR-3943, miR-490-3p, miR-383-5p, miR-133b, miR-129-2-3p, miR-128-2-5p, miR-133a-3p, miR-129-5p, miR-1-3p, miR-885-3p, miR-124-5p, miR-759, miR-7158-3p, miR-770-5p, miR-135a-5p, miR-885-5p, let-7g-5p, miR-100, miR-101, miR-106a, miR-124, miR-124a, miR-125a, miR-125a-5p, miR-125b, miR-127-3p, miR-128, miR-129, miR-136, miR-137, miR-139-5p, miR-142-3p, miR-143, miR-145, miR-146b-5p, miR-149, miR-152, miR-153, miR-195, miR-21, miR-212-3p, miR-219-5p, miR-222, miR-29b, miR-31, miR-3189-3p, miR-320, miR-320a, miR-326, miR-330, miR-331-3p, miR-340, miR-342, miR-34a, miR-376a, miR-449a, miR-483-5p, miR-503, miR-577, miR-663, miR-7, miR-7-5p, miR-873, let-7a, let-7f, miR-107, miR-122, miR-124-5p, miR-139, miR-146a, miR-146b, miR-15b, miR-16, miR-181a, miR-181a-1, miR-181a-2, miR-181b, miR-181b-1, miR-181b-2, miR-181c, miR-181d, miR-184, miR-185, miR-199a-3p, miR-200a, miR-200b, miR-203, miR-204, miR-205, miR-218, miR-23b, miR-26b, miR-27a, miR-29c, miR-328, miR-34c-3p, miR-34c-5p, miR-375, miR-383, miR-451, miR-452, miR-495, miR-584, miR-622, miR-656, miR-98, miR-124-3p, miR-181b-5p, miR-200b, and/or miR-3189-3p.

23. The oncolytic virus of claim 22 for treating brain cancer.

24. The oncolytic virus of any of claims 1-12, wherein the tumor-suppressive miR target sequence is a target sequence for miR-10b-5p, miR-126-3p, miR-145-3p, miR-451a, miR-199b-5p, miR-5683, miR-3195, miR-3182, miR-1271-5p, miR-204-5p, miR-409-5p, miR-136-5p, miR-514a-5p, miR-559, miR-483-3p, miR-1-3p, miR-6080, miR-144-3p, miR-10b-3p, miR-6130, miR-6089, miR-203b-5p, miR-4266, miR-4327, miR-5694, miR-193b, let-7a, let-7a-1, let-7a-2, let-7a-3, let-7b, let-7c, let-7d, let-7e, let-7f-1, let-7f-2, let-7g, let-7i, miR-100, miR-107, miR-10a, miR-10b, miR-122, miR-124, miR-1258, miR-125a-5p, miR-125b, miR-126, miR-127, miR-129, miR-130a, miR-132, miR-133a, miR-143, miR-145, miR-146a, miR-146b, miR-147, miR-148a, miR-149, miR-152, miR-153, miR-15a, miR-16, miR-17-5p, miR-181a, miR-1826, miR-183, miR-185, miR-191, miR-193a-3p, miR-195, miR-199b-5p, miR-19a-3p, miR-200a, miR-200b, miR-200c, miR-205, miR-206, miR-211, miR-216b, miR-218, miR-22, miR-26a, miR-26b, miR-300, miR-30a, miR-31, miR-335, miR-339-5p, miR-33b, miR-34a, miR-34b, miR-34c, miR-374a, miR-379, miR-381, miR-383, miR-425, miR-429, miR-450b-3p, miR-494, miR-495, miR-497, miR-502-5p, miR-517a, miR-574-3p, miR-638, miR-7, miR-720, miR-873, miR-874, miR-92a, miR-98, miR-99a, mmu-miR-290-3p, and/or mmu-miR-290-5p.

25. The oncolytic virus of claim 24 for treating breast cancer.

26. The oncolytic virus of any of claims 1-12, wherein the tumor-suppressive miR target sequence is a target sequence for miR-143, miR-145, miR-17-5p, miR-203, miR-214, miR-218, miR-335, miR-342-3p, miR-372, miR-424, miR-491-5p, miR-497, miR-7, miR-99a, miR-99b, miR-100, miR-101, miR-15a, miR-16, miR-34a, miR-886-5p, miR-106a, miR-124, miR-148a, miR-29a, and/or miR-375.

27. The oncolytic virus of claim 26 for treating cervical cancer.

28. The oncolytic virus of any of claims 1-12, wherein the tumor-suppressive miR target sequence is a target sequence for miR-133a-5p, miR-490-5p, miR-124-3p, miR-137, miR-655-3p, miR-376c-3p, miR-369-5p, miR-490-3p, miR-432-5p, miR-487b-3p, miR-342-3p, miR-223-3p, miR-136-3p, miR-136-3p, miR-143-5p, miR-1-3p, miR-214-3p, miR-143-3p, miR-199a-3p, miR-199b-3p, miR-451a, miR-127-3p, miR-133a-3p, miR-145-5p, miR-145-3p, miR-199a-5p, let-7a-1, let-7a-2, let-7a-3, let-7b, let-7c, let-7d, let-7e, let-7f-1, let-7f-2, let-7g, let-7i, miR-100, miR-101, miR-126, miR-142-3p, miR-143, miR-145, miR-192, miR-200c, miR-21, miR-214, miR-215, miR-22, miR-25, miR-302a, miR-320, miR-320a, miR-34a, miR-34c, miR-365, miR-373, miR-424, miR-429, miR-455, miR-484, miR-502, miR-503, miR-93, miR-98, miR-186, miR-30a-5p, miR-627, let-7a, miR-1, miR-124, miR-125a, miR-129, miR-1295b-3p, miR-1307, miR-130b, miR-132, miR-133a, miR-133b, miR-137, miR-138, miR-139, miR-139-5p, miR-140-5p, miR-148a, miR-148b, miR-149, miR-150-5p, miR-154, miR-15a, miR-15b, miR-16, miR-18a, miR-191, miR-193a-5p, miR-194, miR-195, miR-196a, miR-198, miR-199a-5p, miR-203, miR-204-5p, miR-206, miR-212, miR-218, miR-224, miR-24-3p, miR-26b, miR-27a, miR-28-3p, miR-28-5p, miR-29b, miR-30a-3p, miR-30b, miR-328, miR-338-3p, miR-342, miR-345, miR-34a-5p, miR-361-5p, miR-375, miR-378, miR-378a-3p, miR-378a-5p, miR-409-3p, miR-422a, miR-4487, miR-483, miR-497, miR-498, miR-518a-3p, miR-551a, miR-574-5p, miR-625, miR-638, miR-7, miR-96-5p, miR-202-3p, miR-30a, and/or miR-451.

29. The oncolytic virus of claim 28 for treating colon or colorectal cancer.

30. The oncolytic virus of any of claims 1-12, wherein the tumor-suppressive miR target sequence is a target sequence for miR-101, miR-130a, miR-130b, miR-134, miR-143, miR-145, miR-152, miR-205, miR-223, miR-301a, miR-301b, miR-30c, miR-34a, miR-34c, miR-424, miR-449a, miR-543, and/or miR-34b inserted into the 5′ UTR or 3′ UTR of one or more viral genes required for viral replication.

31. The oncolytic virus of claim 30 for treating endometrial cancer.

32. The oncolytic virus of any of claims 1-12, wherein the tumor-suppressive miR target sequence is a target sequence for miR-125b, miR-138, miR-15a, miR-15b, miR-16, miR-16-1, miR-16-1-3p, miR-16-2, miR-181a, miR-181b, miR-195, miR-223, miR-29b, miR-34b, miR-34c, miR-424, miR-10a, miR-146a, miR-150, miR-151, miR-155, miR-2278, miR-26a, miR-30e, miR-31, miR-326, miR-564, miR-27a, let-7b, miR-124a, miR-142-3p, let-7c, miR-17, miR-20a, miR-29a, miR-30c, miR-720, miR-107, miR-342, miR-34a, miR-202, miR-142-5p, miR-29c, miR-145, miR-193b, miR-199a, miR-214, miR-22, miR-137, and/or miR-197 inserted into the 5′ UTR or 3′ UTR of one or more viral genes required for viral replication.

33. The oncolytic virus of claim 32 for treating hematologic cancer.

34. The oncolytic virus of any of claims 1-12, wherein the tumor-suppressive miR target sequence is a target sequence for miR-1, miR-145, miR-1826, miR-199a, miR-199a-3p, miR-203, miR-205, miR-497, miR-508-3p, miR-509-3p, let-7a, let-7d, miR-106a*, miR-126, miR-1285, miR-129-3p, miR-1291, miR-133a, miR-135a, miR-138, miR-141, miR-143, miR-182-5p, miR-200a, miR-218, miR-28-5p, miR-30a, miR-30c, miR-30d, miR-34a, miR-378, miR-429, miR-509-5p, miR-646, miR-133b, let-7b, let-7c, miR-200c, miR-204, miR-335, miR-377, and/or miR-506 inserted into the 5′ UTR or 3′ UTR of one or more viral genes required for viral replication.

35. The oncolytic virus of claim 34 for treating kidney cancer.

36. The oncolytic virus of any of claims 1-12, wherein the tumor-suppressive miR target sequence is a target sequence for let-7a-1, let-7a-2, let-7a-3, let-7b, let-7c, let-7d, let-7e, let-7f, let-7f-1, let-7f-2, let-7g, let-7i, miR-1, miR-100, miR-101, miR-105, miR-122, miR-122a, miR-1236, miR-124, miR-125b, miR-126, miR-127, miR-1271, miR-128-3p, miR-129-5p, miR-130a, miR-130b, miR-133a, miR-134, miR-137, miR-138, miR-139, miR-139-5p, miR-140-5p, miR-141, miR-142-3p, miR-143, miR-144, miR-145, miR-146a, miR-148a, miR-148b, miR-150-5p, miR-15b, miR-16, miR-181a-5p, miR-185, miR-188-5p, miR-193b, miR-195, miR-195-5p, miR-197, miR-198, miR-199a, miR-199a-5p, miR-199b, miR-199b-5p, miR-200a, miR-200b, miR-200c, miR-202, miR-203, miR-204-3p, miR-205, miR-206, miR-20a, miR-21, miR-21-3p, miR-211, miR-212, miR-214, miR-217, miR-218, miR-219-5p, miR-22, miR-223, miR-26a, miR-26b, miR-29a, miR-29b-1, miR-29b-2, miR-29c, miR-302b, miR-302c, miR-30a, miR-30a-3p, miR-335, miR-338-3p, miR-33a, miR-34a, miR-34b, miR-365, miR-370, miR-372, miR-375, miR-376a, miR-377, miR-422a, miR-424, miR-424-5p, miR-433, miR-4458, miR-448, miR-450a, miR-451, miR-485-5p, miR-486-5p, miR-497, miR-503, miR-506, miR-519d, miR-520a, miR-520b, miR-520c-3p, miR-582-5p, miR-590-5p, miR-610, miR-612, miR-625, miR-637, miR-675, miR-7, miR-877, miR-940, miR-941, miR-98, miR-99a, miR-132, and/or miR-31 inserted into the 5′ UTR or 3′ UTR of one or more viral genes required for viral replication.

37. The oncolytic virus of claim 36 for treating liver cancer.

38. The oncolytic virus of claim 37, wherein the liver cancer is hepatocellular carcinoma.

39. The oncolytic virus of any of claims 1-12, wherein the tumor-suppressive miR target sequence is a target sequence for miR-143-3p, miR-126-3p, miR-126-5p, miR-1266-3p, miR-6130, miR-6080, miR-511-5p, miR-143-5p, miR-223-5p, miR-199b-5p, miR-199a-3p, miR-199b-3p, miR-451a, miR-142-5p, miR-144, miR-150-5p, miR-142-3p, miR-214-3p, miR-214-5p, miR-199a-5p, miR-145-3p, miR-145-5p, miR-1297, miR-141, miR-145, miR-16, miR-200a, miR-200b, miR-200c, miR-29b, miR-381, miR-409-3p, miR-429, miR-451, miR-511, miR-99a, let-7a-1, let-7a-2, let-7a-3, let-7b, let-7c, let-7d, let-7e, let-7f-1, let-7f-2, let-7g, let-7i, miR-1, miR-101, miR-133b, miR-138, miR-142-5p, miR-144, miR-1469, miR-146a, miR-153, miR-15a, miR-15b, miR-16-1, miR-16-2, miR-182, miR-192, miR-193a-3p, miR-194, miR-195, miR-198, miR-203, miR-217, miR-218, miR-22, miR-223, miR-26a, miR-26b, miR-29c, miR-33a, miR-34a, miR-34b, miR-34c, miR-365, miR-449a, miR-449b, miR-486-5p, miR-545, miR-610, miR-614, miR-630, miR-660, miR-7515, miR-9500, miR-98, miR-99b, miR-133a, let-7a, miR-100, miR-106a, miR-107, miR-124, miR-125a-3p, miR-125a-5p, miR-126, miR-126*, miR-129, miR-137, miR-140, miR-143, miR-146b, miR-148a, miR-148b, miR-149, miR-152, miR-154, miR-155, miR-17-5p, miR-181a-1, miR-181a-2, miR-181b, miR-181b-1, miR-181b-2, miR-181c, miR-181d, miR-184, miR-186, miR-193b, miR-199a, miR-204, miR-212, miR-221, miR-224, miR-27a, miR-27b, miR-29a, miR-30a, miR-30b, miR-30c, miR-30d, miR-30d-5p, miR-30e-5p, miR-32, miR-335, miR-338-3p, miR-340, miR-342-3p, miR-361-3p, miR-373, miR-375, miR-4500, miR-4782-3p, miR-497, miR-503, miR-512-3p, miR-520a-3p, miR-526b, miR-625*, and/or miR-96 inserted into the 5′ UTR or 3′ UTR of one or more viral genes required for viral replication.

40. The oncolytic virus of claim 39 for treating lung cancer.

41. The oncolytic virus of any of claims 1-12, wherein the tumor-suppressive miR target sequence is a target sequence for let-7b, miR-101, miR-125b, miR-1280, miR-143, miR-146a, miR-146b, miR-155, miR-17, miR-184, miR-185, miR-18b, miR-193b, miR-200c, miR-203, miR-204, miR-205, miR-206, miR-20a, miR-211, miR-218, miR-26a, miR-31, miR-33a, miR-34a, miR-34c, miR-376a, miR-376c, miR-573, miR-7-5p, miR-9, and/or miR-98 inserted into the 5′ UTR or 3′ UTR of one or more viral genes required for viral replication.

42. The oncolytic virus of claim 41 for treating melanoma.

43. The oncolytic virus of any of claims 1-12, wherein the tumor-suppressive miR target sequence is a target sequence for let-7d, miR-218, miR-34a, miR-375, miR-494, miR-100, miR-124, miR-1250, miR-125b, miR-126, miR-1271, miR-136, miR-138, miR-145, miR-147, miR-148a, miR-181a, miR-206, miR-220a, miR-26a, miR-26b, miR-29a, miR-32, miR-323-5p, miR-329, miR-338, miR-370, miR-410, miR-429, miR-433, miR-499a-5p, miR-503, miR-506, miR-632, miR-646, miR-668, miR-877, and/or miR-9 inserted into the 5′ UTR or 3′ UTR of one or more viral genes required for viral replication.

44. The oncolytic virus of claim 43 for treating oral cancer.

45. The oncolytic virus of any of claims 1-12, wherein the tumor-suppressive miR target sequence is a target sequence for let-7i, miR-100, miR-124, miR-125b, miR-129-5p, miR-130b, miR-133a, miR-137, miR-138, miR-141, miR-145, miR-148a, miR-152, miR-153, miR-155, miR-199a, miR-200a, miR-200b, miR-200c, miR-212, miR-335, miR-34a, miR-34b, miR-34c, miR-409-3p, miR-411, miR-429, miR-432, miR-449a, miR-494, miR-497, miR-498, miR-519d, miR-655, miR-9, miR-98, miR-101, miR-532-5p, miR-124a, miR-192, miR-193a, and/or miR-7 inserted into the 5′ UTR or 3′ UTR of one or more viral genes required for viral replication.

46. The oncolytic virus of claim 45 for treating ovarian cancer.

47. The oncolytic virus of any of claims 1-12, wherein the tumor-suppressive miR target sequence is a target sequence for miR-216a-5p, miR-802, miR-217, miR-145-3p, miR-143-3p, miR-451a, miR-375, miR-214-3p, miR-216b-3p, miR-432-5p, miR-216a-3p, miR-199b-5p, miR-199a-5p, miR-136-3p, miR-216b-5p, miR-136-5p, miR-145-5p, miR-127-3p, miR-199a-3p, miR-199b-3p, miR-559, miR-129-2-3p, miR-4507, miR-1-3p, miR-148a-3p, miR-101, miR-1181, miR-124, miR-1247, miR-133a, miR-141, miR-145, miR-146a, miR-148a, miR-148b, miR-150*, miR-150-5p, miR-152, miR-15a, miR-198, miR-203, miR-214, miR-216a, miR-29c, miR-335, miR-34a, miR-34b, miR-34c, miR-373, miR-375, miR-410, miR-497, miR-615-5p, miR-630, miR-96, miR-132, let-7a, let-7a-1, let-7a-2, let-7a-3, let-7b, let-7c, let-7d, let-7e, let-7f-1, let-7f-2, let-7g, let-7i, miR-126, miR-135a, miR-143, miR-144, miR-150, miR-16, miR-200a, miR-200b, miR-200c, miR-217, miR-218, miR-337, miR-494, and/or miR-98 inserted into the 5′ UTR or 3′ UTR of one or more viral genes required for viral replication.

48. The oncolytic virus of claim 47 for treating pancreatic cancer.

49. The oncolytic virus of any of claims 1-12, wherein the tumor-suppressive miR target sequence is a target sequence for let-7a-3p, let-7c, miR-100, miR-101, miR-105, miR-124, miR-128, miR-1296, miR-130b, miR-133a-1, miR-133a-2, miR-133b, miR-135a, miR-143, miR-145, miR-146a, miR-154, miR-15a, miR-187, miR-188-5p, miR-199b, miR-200b, miR-203, miR-205, miR-212, miR-218, miR-221, miR-224, miR-23a, miR-23b, miR-25, miR-26a, miR-26b, miR-29b, miR-302a, miR-30a, miR-30b, miR-30c-1, miR-30c-2, miR-30d, miR-30e, miR-31, miR-330, miR-331-3p, miR-34a, miR-34b, miR-34c, miR-374b, miR-449a, miR-4723-5p, miR-497, miR-628-5p, miR-642a-5p, miR-765, and/or miR-940 inserted into the 5′ UTR or 3′ UTR of one or more viral genes required for viral replication.

50. The oncolytic virus of claim 49 for treating prostate cancer.

51. The oncolytic virus of any of claims 1-12, wherein the tumor-suppressive miR target sequence is a target sequence for miR-101, miR-183, miR-204, miR-34a, miR-365b-3p, miR-486-3p, and/or miR-532-5p inserted into the 5′ UTR or 3′ UTR of one or more viral genes required for viral replication.

52. The oncolytic virus of claim 51 for treating retinoblastoma.

53. The oncolytic virus of any of claims 1-12, wherein the tumor-suppressive miR target sequence is a target sequence for miR-143-3p, miR-133b, miR-1264, miR-448, miR-1298-5p, miR-490-5p, miR-138-2-3p, miR-144-3p, miR-144-5p, miR-150-5p, miR-129-1-3p, miR-559, miR-1-3-p, miR-143-5p, miR-223-3p, miR-3943, miR-338-3p, miR-124-3p, miR-219a-5p, miR-219a-2-3p, miR-451a, miR-142-5p, miR-133a-3p, miR-145-5p, and/or miR-145-3p.

54. The oncolytic virus of claim 53 for treating glioblastoma.

55. The oncolytic virus of any of claims 1-12, wherein the tumor-suppressive miR target sequence is a target sequence for miR-143-3p, miR-223-3p, miR-6080, miR-208b-3p, miR-206, miR-133a-5p, miR-133b, miR-199a-5p, miR-199b-5p, miR-145-3p, miR-145-5p, miR-150-5p, miR-142-3p, miR-144-3p, miR-144-5p, miR-338-3p, miR-214-3p, miR-559, miR-133a-3p, miR-1-3p, miR-126-3p, miR-142-5p, miR-451a, miR-199a-3p, and/or miR-199b-3p.

56. The oncolytic virus of claim 55 for treating head and neck cancer.

57. The oncolytic virus of any of claims 1-12, wherein the tumor-suppressive miR target sequence is a target sequence for miR-133b, miR-208b-3p, miR-6130, miR-141-5p, miR-31-3p, miR-1293, miR-129-2-3p, miR-129-5p, miR-124-3p, miR-219a-5p, miR-219a-2-3p, miR-490-3p, miR-488-3p, miR-935, miR-124-5p, miR-122-3p, miR-122-5p, miR-1-3p, miR-133a-3p, miR-375, miR-141-3p, miR-31-5p, miR-205-5p, miR-200c-3p, and/or miR-203a-3p.

58. The oncolytic virus of claim 57 for treating a Schwannoma.

59. A recombinant oncolytic virus comprising one or more of;
(a) one or more tumor-suppressive micro-RNA (miR) target sequences inserted into a locus of one or more viral genes required for viral replication;
(b) one or more polynucleotides encoding one or more proteins or oligonucleotides, wherein the proteins or oligonucleotides reduce the expression or inhibit the function of a miR, a gene, or a TIMP;
(c) at least one protease-activated antibody; and/or
(d) a polynucleotide encoding at least one protease activated antibody.

60. The oncolytic virus of claim 59, wherein the miR of (b) is an oncogenic miR or a microenvironment remodeling miR.

61. The oncolytic virus of claim 60, wherein the oncogenic miR is selected from the miRs listed in Table 4.

62. The oncolytic virus of claim 59, wherein the gene of (b) is an oncogenic gene.

63. The oncolytic virus of claim 62, wherein the oncogenic gene is selected from the genes listed in Table 7.

64. The oncolytic virus of claim 60, wherein the microenvironment remodeling miR is selected from the miRs listed in Table 5.

65. The oncolytic virus of claim 59, wherein the TIMP of (b) is selected from TIMP1, TIMP2, TIMP3 and TIMP4.

66. The oncolytic virus of claim 59, wherein the oligonucleotide of (b) is an shRNA or a decoy oligonucleotide.

67. The oncolytic virus of claim 59, wherein the protein of (b) is a nuclease, a bispecific T-cell engager (BiTE), an anti-immunosuppressive protein, or an immunogenic antigen.

68. The oncolytic virus of claim 67, wherein the nuclease is selected from a Clustered Regulatory Interspaced Short Palindromic Repeats (CRISPR)-associated endonuclease, a zinc-finger nuclease (ZFN) or a Transcription activator-like effector nuclease (TALEN).

69. The oncolytic virus of claim 68, wherein the CRISPR-associated endonuclease is selected from SpCas9, SpCas9-HF1, SpCas9-HF2, SpCas9-HF3, SpCas9-HF4, SaCas9, FnCpf, FnCas9, eSpCas9, C2C1, C2C3, Cpf1, Casl, Cas1B, Cas2, Cas3, Cas4, Cas5, Cas6, Cas7, Cas8, Cas9, Cas10, Csyl, Csy2, Csy3, Csel, Cse2, Cscl, Csc2, Csa5, Csn2, Csm2, Csm3, Csm4, Csm5, Csm6, Cmrl, Cmr3, Cmr4, Cmr5, Cmr6, Csbl, Csb2, Csb3, Csxl7, Csxl4, Csx10, Csx16, CsaX, Csx3, Csxl, Csxl5, Csfl, Csf2, Csf3, and Csf4.

70. The oncolytic virus of claim 69, further comprising a heterologous polynucleotide encoding an tracr-RNA (trRNA) and a crispr-RNA (crRNA), wherein the crRNA is targeted to a genomic DNA sequence encoding a miR or a TIMP and wherein the trRNA facilitates binding and activation of a CRISPR-associated endonuclease

71. The oncolytic virus of claim 67, wherein the anti-immunosuppressive protein is an anti-regulatory T-cell (Treg) protein or an anti-myeloid-derived suppressor cell (MDSC) protein.

72. The oncolytic virus of claim 67, wherein the anti-immunosuppressive protein is a VHH-derived blocker or a VHH-derived BiTE.

73. The oncolytic virus of claim 59, wherein the protein of (b) induces an anti-tumor immune response.

74. The oncolytic virus of claim 73, wherein the protein is selected from EpCAM, folate, IFNβ, anti-CTLA-4, anti-PD1, A2A, anti-FGF2, anti-FGFR/FGFR2b, anti-SEMA4D, CCL5, CD137, CD200, CD38, CD44, CSF-1R, CXCL10, CXCL13, endothelin B Receptor, IL-12, IL-15, IL-2, IL-21, IL-35, ISRE7, LFA-1, NG2 (also known as SPEG4), a SMAD protein, STING, TGFβ, and VCAM1.

75. The oncolytic virus of claim 59, wherein the at least one protease-activated antibody of (c) or (d) is incorporated into a viral glycoprotein envelope.

76. The oncolytic virus of claim 59 or 75, wherein the protease-activated antibody is activated by a protease selected from a cysteine cathepsin, an aspartic cathepsin, a kallikrein (hK), a serine protease, a caspase, a matrix metalloproteinase (MMP), and a disintegrin metalloproteinase (ADAM).

77. The oncolytic virus of claim 76, wherein the protease is selected from cathepsin K, cathepsin B, cathepsin L, cathepsin E, cathepsin D, hK1, PSA (hK3), hK10, hK15, uPA, uPAR, MMP-1, MMP-2, MMP-3, MMP-7, MMP-8, MMP-9, MMP-10, MMP-11, MMP-12, MMP-13, MMP-14, MMP-15, MMP-16, MMP-17, MMP-18, MMP-19, MMP-20, MMP-21, MMP-23A, MMP-23B, MMP-24, MMP-25, MMP-26, MMP-27, MMP-28, or a protease listed in Table 6.

78. The oncolytic virus of claim 59 or 75, wherein the protease-activated antibody binds to a protein expressed more highly by a cancer cell or in a cancer microenvironment than by a non-cancer cell or in a non-cancer microenvironment.

79. The oncolytic virus of claim 78, wherein the protease-activated antibody binds NKG2D, c-met, HGFR, CD8, heparan sulfate, VSPG4 (also known as NG2), EGFR, EGFRvIII, CD133, CXCR4, carcinoembryonic antigen (CEA), CLC-3, annexin II, human transferrin receptor, or EpCAM.

80. The oncolytic virus of any of claims 59-79, wherein the miR target sequence of (a), the one or more polynucleotides of (b), and/or or the one or more polynucleotides of (d) is inserted into a gene locus of the viral genome.

81. The oncolytic virus of claim 80, wherein the virus is a herpes simplex virus and wherein the at least one polynucleotide is inserted into or between one or more viral gene loci selected from the group consisting of the internal repeat joint region (comprising one copy each of the diploid genes ICPO, ICP34.5, LAT, ICP4, and the ICP47 promoter), ICPO, LAT, UL1, UL5, UL6, UL7, UL8, UL9, UL11, UL12, UL14, UL15, UL17, UL18, UL19, UL20, UL22, UL25, UL26, UL26.5, UL27, UL28, UL29, UL30, UL31, UL32, UL33, UL34, UL35, UL36, UL37, UL38, UL39, UL40, UL42, UL48, UL49, UL52, UL53, UL54, ICPO, ICP4, ICP22, ICP27, ICP47, gamma-34.5, US3, US4, US5, US6, US7, US8, US9, US10, US11, and US12.

82. A nucleic acid molecule encoding the oncolytic virus of any of the preceding claims.

83. A viral stock comprising the oncolytic virus of any of claims 1-81.

84. A composition comprising the oncolytic virus of any of claims 1-81 and a pharmaceutically-acceptable carrier.

85. A method of killing a cancerous cell, comprising exposing the cancerous cell to the oncolytic virus of any of claims 1-81 or compositions thereof under conditions sufficient for the oncolytic virus to infect and replicate within said cancerous cell, and wherein replication of the oncolytic virus within the cancerous cell results in cell death.

86. The method of claim 85, wherein the cancerous cell has a reduced expression of a tumor-suppressive miR capable of binding to the one or more tumor-suppressive miR-target sequences compared to the expression of the tumor-suppressive miR in a non-cancerous cell.

87. The oncolytic virus of claim 86, wherein the expression level of the tumor-suppressive miR in the cancerous cell is at least 5% less than the expression level the tumor-suppressive miR in the non-cancerous cell.

88. The method of any of claims 85-87, wherein replication of the oncolytic virus is increased or maintained in cancerous cells with a reduced expression of the tumor-suppressive miR capable of binding to the one or more tumor-suppressive miR-target sequences.

89. The method of claim 88, wherein the viral replication is at least 5% greater in the cancerous cells compared to the viral replication in the non-cancerous cell.

90. The method of any of claims 85-89, wherein the cell is in vivo.

91. The method of claim 90, wherein the cell is within a tumor.

92. A method of treating cancer in a subject in need thereof, comprising administering the oncolytic virus of any of claims 1-81 or compositions thereof to the subject.

93. The method of any one of claims 85-92, wherein the subject is a mouse, a rat, a rabbit, a cat, a dog, a horse, a non-human primate, or a human.

94. The method of claim 92 or 93, wherein the oncolytic virus or compositions thereof are administered intravenously, subcutaneously, intratumorally, intramuscularly, or intranasally.

95. The method of claim 92, wherein the cancer is selected from lung cancer, breast cancer, ovarian cancer, cervical cancer, prostate cancer, testicular cancer, colorectal cancer, colon cancer, pancreatic cancer, liver cancer, gastric cancer, head and neck cancer, thyroid cancer, malignant glioma, glioblastoma, melanoma, B-cell chronic lymphocytic leukemia, diffuse large B-cell lymphoma (DLBCL), and marginal zone lymphoma (MZL).

96. The method of claim 95, wherein the lung cancer is small cell lung cancer or non-small cell lung cancer.

97. The method of claim 96, wherein the liver cancer is hepatocellular carcinoma (HCC).


SEQUENCE LISTING (41 sequences)

SEQ ID NO: 1 - RNA, 22 nt, synthetic construct
  source 1..22: mol_type = other RNA
acagttcttcaactggcagctt

SEQ ID NO: 2 - RNA, 19 nt, synthetic construct
  source 1..19: mol_type = other RNA
ccacgacctaatcctaact

SEQ ID NO: 3 - RNA, 21 nt, synthetic construct
  source 1..21: mol_type = other RNA
gggagctaggttgccatgtgc

SEQ ID NO: 4 - RNA, 23 nt, synthetic construct
  source 1..23: mol_type = other RNA
ctacctgcactgtaagcactttg

SEQ ID NO: 5 - RNA, 22 nt, synthetic construct
  source 1..22: mol_type = other RNA
ctagtggtcctaaacatttcac

SEQ ID NO: 6 - RNA, 20 nt, synthetic construct
  source 1..20: mol_type = other RNA
atttagtgtgataatggcgt

SEQ ID NO: 7 - RNA, 22 nt, synthetic construct
  source 1..22: mol_type = other RNA
caaacaccattgtcacactcca

SEQ ID NO: 8 - RNA, 21 nt, synthetic construct
  source 1..21: mol_type = other RNA
aagctgccagttgaagaactg

SEQ ID NO: 9 - RNA, 20 nt, synthetic construct
  source 1..20: mol_type = other RNA
gcacatggcaacctagctcc

SEQ ID NO: 10 - RNA, 22 nt, synthetic construct
  source 1..22: mol_type = other RNA
aaagtgcttacagtgcaggtag

SEQ ID NO: 11 - RNA, 20 nt, synthetic construct
  source 1..20: mol_type = other RNA
acgccattatcacactaaat

SEQ ID NO: 12 - RNA, 177 nt, synthetic construct
  source 1..177: mol_type = other RNA
gagatcatatgcatatgtgtagggctggagcgtgtgtgtgtcttgagattgtgtgtgttgcagtcatcatatctatgtgttacagattgtgtatgttagccttgtgtatgtgtgcttgattgaggtggtgtatttgggttgaaattgtgtcatatgtgtgtgctatccatctcgtgt

SEQ ID NO: 13 - RNA, 180 nt, synthetic construct
  source 1..180: mol_type = other RNA
gcagaagaggtaagggagtgaggcactcctatcccagtctcccaggtttggttgagggctccccaaggcagggcaagatagcggccctgtcactgaccctggcctgtggtggtctgagctggggagggaaggacaccaatgaatcagcttgggacctctttaggccttccccttttcctc

SEQ ID NO: 14 - RNA, 228 nt, synthetic construct
  source 1..228: mol_type = other RNA
gcattaaaacacacatacaaacaaaatcaaaaacactgcggactttcactcaagctggtctttcttccccagtgtaaggcaatcctgcctactaacaacaccaacaacaaaacactccatctgtgaaagctgacgcagttaagggggctaggcagggcatttgtgccaactaagaatcaccagatacccaccataagtacctatcgcagttttgaagtcgtttctccc

SEQ ID NO: 15 - RNA, 177 nt, synthetic construct
  source 1..177: mol_type = other RNA
caaccatcacgtcaggctgcccatccaatagactcctgggatggggcagccaaccctggctcatctcatctgccgcttggtgcgtgtgcgtgtgcgtgcatgtgcgtgtgcgtgtgtgcaggggtgagaatctggcagatggtgcctctgcctgctcttcttcgcctcctttattta

SEQ ID NO: 16 - RNA, 152 nt, synthetic construct
  source 1..152: mol_type = other RNA
tggccctgcgcatgctgaaataactggaacccagcctctcctcctacaccggcctacccatctgggcccaagagctgcactcacactcctacaacgaaggacaaactgtccaggtcggagggatcacgagacacagaacctggaggggtgtg

SEQ ID NO: 17 - RNA, 227 nt, synthetic construct
  source 1..227: mol_type = other RNA
tctgacttgagagctcccccagtcagatctcaggcttgtccccctgtcagctgcctccagaagggaaggtagccagtgcctgagaagacagtcccttttctacccaccgcactccataacctccatcttctcccacactgatggcgagcagcccctgagcactttctgggactgggagactgcttggtgttccctgaggacaagagacatcctgacagtgttgggca

SEQ ID NO: 18 - DNA, 22 bp, synthetic construct
  source 1..22: mol_type = other DNA
acagttcttcaactggcagctt

SEQ ID NO: 19 - DNA, 19 bp, synthetic construct
  source 1..19: mol_type = other DNA
ccacgacctaatcctaact

SEQ ID NO: 20 - DNA, 21 bp, synthetic construct
  source 1..21: mol_type = other DNA
gggagctaggttgccatgtgc

SEQ ID NO: 21 - DNA, 23 bp, synthetic construct
  source 1..23: mol_type = other DNA
ctacctgcactgtaagcactttg

SEQ ID NO: 22 - DNA, 22 bp, synthetic construct
  source 1..22: mol_type = other DNA
ctagtggtcctaaacatttcac

SEQ ID NO: 23 - DNA, 20 bp, synthetic construct
  source 1..20: mol_type = other DNA
atttagtgtgataatggcgt

SEQ ID NO: 24 - DNA, 22 bp, synthetic construct
  source 1..22: mol_type = other DNA
caaacaccattgtcacactcca

SEQ ID NO: 25 - DNA, 21 bp, synthetic construct
  source 1..21: mol_type = other DNA
aagctgccagttgaagaactg

SEQ ID NO: 26 - DNA, 20 bp, synthetic construct
  source 1..20: mol_type = other DNA
gcacatggcaacctagctcc

SEQ ID NO: 27 - DNA, 22 bp, synthetic construct
  source 1..22: mol_type = other DNA
aaagtgcttacagtgcaggtag

SEQ ID NO: 28 - DNA, 20 bp, synthetic construct
  source 1..20: mol_type = other DNA
acgccattatcacactaaat

SEQ ID NO: 29 - DNA, 177 bp, synthetic construct
  source 1..177: mol_type = other DNA
gagatcatatgcatatgtgtagggctggagcgtgtgtgtgtcttgagattgtgtgtgttgcagtcatcatatctatgtgttacagattgtgtatgttagccttgtgtatgtgtgcttgattgaggtggtgtatttgggttgaaattgtgtcatatgtgtgtgctatccatctcgtgt

SEQ ID NO: 30 - DNA, 180 bp, synthetic construct
  source 1..180: mol_type = other DNA
gcagaagaggtaagggagtgaggcactcctatcccagtctcccaggtttggttgagggctccccaaggcagggcaagatagcggccctgtcactgaccctggcctgtggtggtctgagctggggagggaaggacaccaatgaatcagcttgggacctctttaggccttccccttttcctc

SEQ ID NO: 31 - DNA, 228 bp, synthetic construct
  source 1..228: mol_type = other DNA
gcattaaaacacacatacaaacaaaatcaaaaacactgcggactttcactcaagctggtctttcttccccagtgtaaggcaatcctgcctactaacaacaccaacaacaaaacactccatctgtgaaagctgacgcagttaagggggctaggcagggcatttgtgccaactaagaatcaccagatacccaccataagtacctatcgcagttttgaagtcgtttctccc

SEQ ID NO: 32 - DNA, 177 bp, synthetic construct
  source 1..177: mol_type = other DNA
caaccatcacgtcaggctgcccatccaatagactcctgggatggggcagccaaccctggctcatctcatctgccgcttggtgcgtgtgcgtgtgcgtgcatgtgcgtgtgcgtgtgtgcaggggtgagaatctggcagatggtgcctctgcctgctcttcttcgcctcctttattta

SEQ ID NO: 33 - DNA, 152 bp, synthetic construct
  source 1..152: mol_type = other DNA
tggccctgcgcatgctgaaataactggaacccagcctctcctcctacaccggcctacccatctgggcccaagagctgcactcacactcctacaacgaaggacaaactgtccaggtcggagggatcacgagacacagaacctggaggggtgtg

SEQ ID NO: 34 - DNA, 227 bp, synthetic construct
  source 1..227: mol_type = other DNA
tctgacttgagagctcccccagtcagatctcaggcttgtccccctgtcagctgcctccagaagggaaggtagccagtgcctgagaagacagtcccttttctacccaccgcactccataacctccatcttctcccacactgatggcgagcagcccctgagcactttctgggactgggagactgcttggtgttccctgaggacaagagacatcctgacagtgttgggca

SEQ ID NO: 35 - RNA, 22 nt, synthetic construct
  source 1..22: mol_type = other RNA
taaagcttgccactgaagaact

SEQ ID NO: 36 - RNA, 21 nt, synthetic construct
  source 1..21: mol_type = other RNA
actgcacaagagcacccagcc

SEQ ID NO: 37 - RNA, 22 nt, synthetic construct
  source 1..22: mol_type = other RNA
ctacaagtgccttcactgcagt

SEQ ID NO: 38 - RNA, 25 nt, synthetic construct
  source 1..25: mol_type = other RNA
aactgttgaactgttaagaaccact

SEQ ID NO: 39 - RNA, 23 nt, synthetic construct
  source 1..23: mol_type = other RNA
ctacctgcacgaacagcactttg

SEQ ID NO: 40 - DNA, 23 bp, synthetic construct
  source 1..23: mol_type = other DNA
ctacctgcacgaacagcactttg

SEQ ID NO: 41 - DNA, 127 bp, synthetic construct
  source 1..127: mol_type = other DNA
gcacctgtctaaaaggcactattccccatacaatggtccaaagtctggcagttaccatccgaccgcgttttctgcggagcccaggatactttcttacttaagcttgtgacggcggtagagcatgaaa